(19) United States
(12) Patent Application Publication
(10) Pub. No.: US 2013/0224191 A1
(43) Pub. Date: Aug. 29, 2013

(54) NOTUM PROTEIN MODULATORS AND METHODS OF USE

(71) Applicants: Robert A. Stull, Alameda, CA (US); Scott J. Dylla, Mountain View, CA (US); Orit Foord, Foster City, CA (US); Monette Aujay, San Francisco, CA (US)

(72) Inventors: Robert A. Stull, Alameda, CA (US); Scott J. Dylla, Mountain View, CA (US); Orit Foord, Foster City, CA (US); Monette Aujay, San Francisco, CA (US)

(21) Appl. No.: 13/819,208

(22) PCT Filed: Aug. 26, 2011
(86) PCT No.: PCT/US11/49458

§ 371 Date: Apr. 30, 2013

Related U.S. Application Data
(60) Provisional application No. 61/377,882, filed on Aug. 27, 2010.
(60) Provisional application No. 61/380,181, filed on Sep. 3, 2010.
(60) Provisional application No. 61/388,552, filed on Sep. 30, 2010.
(60) Provisional application No. 61/510,413, filed on Jul. 21, 2011.

(51) Int. Cl. C07K 16/40; G01N 33/68

(52) U.S. Cl. CPC C07K 16/40; G01N 33/6893
USPC 424/133.1; 530/389.1; 530/391.7; 530/387.3; 424/158.1; 424/178.1; 435/7.92; 435/252.33; 435/252.31; 435/252.35; 435/254.21; 435/254.23; 435/419; 435/412; 435/417; 435/338; 435/328; 252/182.3

(57) ABSTRACT

Novel modulators, including antibodies and derivatives thereof, and methods of using such modulators to treat hyperproliferative disorders are provided.

### CROSS REFERENCED APPLICATIONS

This application claims the benefit under 35 U.S.C. 119(e) of U.S. Provisional Application Nos. 61/377,882 filed Aug. 27, 2010, 61/380,181 filed Sep. 3, 2010, 61/388,552 filed Sep. 30, 2010, and 61/510,413 filed Jul. 21, 2011, all of which are incorporated herein by reference in their entirety.

### FIELD OF THE INVENTION

This application generally relates to compositions and methods of their use in treating or ameliorating hyperproliferative disorders, their expansion, recurrence, relapse or metastasis. In a broad aspect the present invention relates to the use of Notum modulators, including Notum antagonists and fusion constructs, for the treatment or prophylaxis of neoplastic disorders. In particularly preferred embodiments the present invention provides for the use of anti-Notum antibodies for the immunotherapeutic treatment of malignancies including, for example, in KRAS and/or APC mutated colorectal cancer and KRAS mutated pancreatic cancers.

### SEQUENCE LISTING

The instant application contains a Sequence Listing which has been submitted in ASCII format via EFS-Web and is hereby incorporated by reference in its entirety. Said ASCII copy, created on Aug. 26, 2011, is named 11200.3.304.txt and is 138,922 bytes in size.

### BACKGROUND OF THE INVENTION

Stem and progenitor cell differentiation and cell proliferation are normal ongoing processes that act in concert to support tissue growth during organogenesis, and cell replacement and repair of most tissues during the lifetime of all living organisms. Differentiation and proliferation decisions are often controlled by numerous factors and signals that are balanced to maintain cell fate decisions and tissue architecture. Normal tissue architecture is maintained as a result of cells responding to microenvironmental cues that regulate cell division and tissue maturation. Accordingly, cell proliferation and differentiation normally occurs only as necessary for the replacement of damaged or dying cells or for growth. Unfortunately, disruption of cell proliferation and/or differentiation can result from a myriad of factors including, for example, the under- or overabundance of various signaling chemicals, the presence of altered microenvironments, genetic mutations or some combination thereof. When normal cellular proliferation and/or differentiation is disturbed or somehow disrupted it can lead to various diseases or disorders including cancer.

Conventional treatments for cancer include chemotherapy, radiotherapy, surgery, immunotherapy (e.g., biological response modifiers, vaccines or targeted therapeutics) or combinations thereof. Sadly, far too many cancers are non-responsive or minimally responsive to such conventional treatments leaving few options for patients. For example, some patient subpopulations exhibit gene mutations (e.g., KRAS,) that render them non-responsive despite the general effectiveness of certain therapies. Moreover, depending on the type of cancer some available treatments, such as surgery, may not be viable alternatives. Limitations inherent in current standard of care therapeutics are particularly evident when attempting to care for patients who have undergone previous treatments and have subsequently relapsed. In such cases the failed therapeutic regimens and resulting patient deterioration may contribute to refractory tumors often manifest themselves as a more aggressive disease that ultimately proves to be incurable. Although there have been great improvements in the diagnosis and treatment of cancer over the years, overall survival rates for many solid tumors have remained largely unchanged due to the failure of existing therapies to prevent relapse, tumor recurrence and metastases. Thus, it remains a challenge to develop more targeted and potent therapies.

One promising area of research involves the use of targeted therapeutics to go after the tumorigenic “seed” cells that appear to underlie many cancers. To that end most solid tissues are now known to contain adult, tissue-resident stem cell populations that generate differentiated cell types that comprise the majority of that tissue. Tumors arising in these tissues similarly consist of heterogeneous populations of cells that also arise from stem cells, but differ markedly in their overall proliferation and organization. While it is increasingly recognized that the majority of tumor cells have a limited ability to proliferate, a minority population of cancer cells (commonly known as cancer stem cells or CSC) have the exclusive ability to extensively self-renew thereby enabling them with tumor reinitiating capacity. More specifically, the cancer stem cell hypothesis proposes that there is a distinct subset of cells (i.e. CSC) within each tumor (approximately 0.1-10%) that is capable of indefinite self-renewal and of generating tumor cells progressively limited in their replication capacity as a result of their differentiation to tumor progenitor cells, and subsequently to terminally differentiated tumor cells.

In recent years it has become more evident these CSC (also known as tumor perpetuating cells or TPC) might be more resistant to traditional chemotherapeutic agents or radiation and thus persist after standard of care clinical therapies to later fuel the growth of relapsing tumors, secondary tumors and metastases. Moreover, there is growing evidence suggests that pathways that regulate organogenesis and/or the self-renewal of normal tissue-resident stem cells are deregulated or altered in CSC, resulting in the continuous expansion of self-renewing cancer cells and tumor formation. See generally Al-Hajj et al., 2004, PMID: 15378087; and Dalerba et al., 2007, PMID: 17548814; each of which is incorporated herein in its entirety by reference. Thus, the effectiveness of traditional, as well as more recent targeted treatment methods, has apparently been limited by the existence and/or emergence of resistant cancer cells that are capable of perpetuating the cancer even in face of these diverse treatment methods. Huff et al., European Journal of Cancer 42: 1293-1297 (2006) and Zhou et al., Nature Reviews Drug Discovery 8: 806-823 (2009) each of which is incorporated herein in its entirety by reference. Such observations are confirmed by the consistent inability of traditional debulking agents to substantially increase patient survival when suffering from solid tumors, and through the development of an increasingly sophisticated understanding as to how tumors grow, recur and metastasize. Accordingly, recent strategies for treating neoplastic disorders have recognized the importance of eliminating, depleting, silencing or promoting the differentiation of tumor perpetuating cells so as to diminish the possibility of tumor recurrence, metastasis or patient relapse.

Efforts to develop such strategies have incorporated recent work involving non-traditional xenograft (NTX) models, wherein primary human solid tumor specimens are implanted and passaged exclusively in immunocompromised mice. Such techniques confirm the existence of a subpopulation of cells with the unique ability to generate heterogeneous tumors and fuel their growth indefinitely. As previously hypothesized, work in NTX models has confirmed that identified CSC subpopulations of tumor cells appear more resistant to debulking regimens such as chemotherapy and radiation, potentially explaining the disparity between clinical response rates and overall survival. Further, employment of NTX models in CSC research has sparked a fundamental change in drug discovery and preclinical evaluation of drug candidates that may lead to CSC-targeted therapies having a major impact on tumor recurrence and metastasis thereby improving patient survival rates. While progress has been made, inherent technical difficulties associated with handling primary and/or xenograft tumor tissue, along with a lack of experimental platforms to characterize CSC identity and differentiation potential, pose major challenges. As such, there remains a substantial need to selectively target cancer stem cells and develop diagnostic, prophylactic or therapeutic compounds or methods that may be used in the treatment, prevention and/or management of hyperproliferative disorders.

### SUMMARY OF THE INVENTION

These and other objectives are provided for by the present invention which, in a broad sense, is directed to methods, compounds, compositions and articles of manufacture that may be used in the treatment of Notum associated disorders (e.g., hyperproliferative disorders or neoplastic disorders). To that end, the present invention provides novel Notum modulators that effectively target cancer stem cells and may be used to treat patients suffering from a wide variety of malignancies. In certain embodiments the disclosed Notum modulators may comprise any compound that recognizes, competes, agonizes, antagonizes, interacts, binds or associates with the Notum polypeptide, its ligand or its gene and modulates, adjusts, alters, changes or modifies the impact of the Notum protein on one or more physiological pathways (e.g., the Wnt/beta-catenin, Hh or BMP pathways). In selected embodiments of the invention, Notum modulators may comprise Notum itself or fragments thereof, either in an isolated form or fused or associated with other moieties (e.g., Fc-Notum, PEG-Notum or Notum associated with a targeting moiety). In other selected embodiments Notum modulators may comprise Notum antagonists which, for the purposes of the instant application, shall be held to mean any construct or compound that recognizes, competes, interacts, binds or associates with Notum and neutralizes, eliminates, reduces, sensitizes, reprograms, inhibits or controls the growth of neoplastic cells including tumor initiating cells. In preferred embodiments the Notum modulators of the instant invention comprise anti-Notum antibodies, or fragments or derivatives thereof, that have unexpectedly been found to silence, neutralize, reduce, decrease, deplete, moderate, diminish, reprogram, eliminate, or otherwise inhibit the ability of tumor initiating cells to propagate, maintain, expand, proliferate or otherwise facilitate the survival, recurrence, regeneration and/or metastasis of neoplastic cells.

In one embodiment the Notum modulator may comprise a humanized antibody wherein said antibody comprises a heavy chain variable region amino acid sequence as set forth in SEQ ID NO: 331 and a light chain variable region amino acid sequence as set forth in SEQ ID NO: 332. In other preferred embodiments the invention will be in the form of a composition comprising hSC2.D2.2 antibody and a pharmaceutically acceptable carrier.

In certain other embodiments the invention will comprise a Notum modulator that reduces the frequency of tumor initiating cells upon administration to a subject. Preferably the reduction in frequency will be determined using in vitro or in vivo limiting dilution analysis. In particularly preferred embodiments such analysis may be conducted using in vivo limiting dilution analysis comprising transplant of live human tumor cells into immunocompromised mice. Alternatively, the limiting dilution analysis may be conducted using in vitro limiting dilution analysis comprising limiting dilution deposition of live human tumor cells into in vitro colony supporting conditions. In either case, the analysis, calculation or quantification of the reduction in frequency will preferably comprise the use of Poisson distribution statistics to provide an accurate accounting. It will be appreciated that, while such quantification methods are preferred, other, less labor intensive methodology such as flow cytometry or immunohistochemistry may also be used to provide the desired values and, accordingly, are expressly contemplated as being within the scope of the instant invention. In such cases the reduction in frequency may be determined using flow cytometric analysis or immunohistochemical detection of tumor cell surface markers known to enrich for tumor initiating cells.

As such, in another preferred embodiment of the instant invention comprises a method of treating a Notum associated disorder comprising administering a therapeutically effective amount of a Notum modulator to a subject in need thereof whereby the frequency of tumor initiating cells is reduced. Again, the reduction in the tumor initiating cell frequency will preferably be determined using in vitro or in vivo limiting dilution analysis.

In this regard it will be appreciated that the present invention is based, at least in part, upon the discovery that the Notum polypeptide is associated with tumor perpetuating cells (i.e., cancer stem cells) that are involved in the etiology of various neoplasia. More specifically, the instant application unexpectedly shows that the administration of various exemplary Notum modulators can reduce, inhibit or eliminate tumorigenic signaling by tumor initiating cells (i.e., reduce the frequency of tumor initiating cells). This reduced signaling, whether by reduction or elimination or reprogramming or silencing of the tumor initiating cells or by modifying tumor cell morphology (e.g., induced differentiation, niche disruption), in turn allows for the more effective treatment of Notum associated disorders by inhibiting tumorigenesis, tumor maintenance, expansion and/or metastasis and recurrence. In other embodiments the disclosed modulators may interfere, suppress or otherwise retard Notum mediated paracrine signaling that may fuel tumor growth. Further, as will be discussed in more detail below, the Notum polypeptide is intimately involved in the Wnt/beta-catenin, hedgehog (Hh) and bone morphogenetic protein (BMP) oncogenic survival pathways. Intervention in these developmental signaling pathways, using the novel Notum modulators described herein, may further ameliorate the disorder by more than one mechanism (i.e., tumor initiating cell reduction and disruption of developmental signaling) to provide an additive or synergistic effect.

Thus, another preferred embodiment of the invention comprises a method of treating a Notum mediated disorder in a subject in need thereof comprising the step of administering a Notum modulator to said subject. In particularly preferred embodiments the Notum modulator will be associated (e.g., conjugated) with an anti-cancer agent. In addition such disruption and collateral benefits may be achieved whether the subject tumor tissue exhibits elevated levels of Notum or reduced or depressed levels of Notum as compared with normal adjacent tissue.

Moreover, there is evidence that the modulators of the instant invention may be especially effective in the treatment of certain solid tumors. As such, in other particularly preferred embodiments the invention comprises a method of treating a subject suffering from neoplastic disorder comprising a solid tumor exhibiting a KRAS mutation, an APC mutation, or a CTNNB1 mutation said method comprising the step of administering a therapeutically effective amount of at least one Notum modulator.

In still other embodiments the present invention comprises a method of inhibiting Notum mediated paracrine signaling in a subject in need thereof comprising the step of administering a pharmaceutically effective amount of a Notum modulator.

Other facets of the instant invention exploit the ability of the disclosed modulators to potentially disrupt multiple oncogenic survival pathways while simultaneously silencing tumor initiating cells. Such multi-active Notum modulators (e.g., Notum antagonists) may prove to be particularly effective when used in combination with standard of care anti-cancer agents or debulking agents. In addition, two or more Notum antagonists (e.g. antibodies that specifically bind to two discrete epitopes on Notum) may be used in combination in accordance with the present teachings. Moreover, as discussed in some detail below, the Notum modulators of the present invention may be used in a conjugated or unconjugated state and, optionally, as a sensitizing agent in combination with a variety chemical or biological anti-cancer agents.

Thus, another preferred embodiment of the instant invention comprises a method of sensitizing a tumor in a subject for treatment with an anti-cancer agent comprising the step of administering a Notum modulator to said subject. In a particularly preferred aspect of the invention the Notum modulator will specifically result in a reduction of tumor initiating cell frequency is as determined using in vitro or in vivo limiting dilution analysis.

Similarly, as the compounds of the instant invention may exert therapeutic benefits through various physiological mechanisms, the present invention is also directed to selected effectors or modulators that are specifically fabricated to exploit certain cellular processes. For example, in certain embodiments the preferred modulator may be engineered to associate with Notum on or near the surface of the tumor initiating cell and stimulate the subject's immune response. In other embodiments the effector may comprise an antibody directed to an epitope that facilitates neutralization of any Notum enzymatic activity which is then used to reduce the amount of Notum substrate in the tumor microenvironment and any associated paracrine signaling. In yet other embodiments the disclosed modulators may act by depleting or eliminating the Notum associated cells. As such, it is important to appreciate that the present invention is not limited to any particular mode of action but rather encompasses any method or Notum modulator that achieves the desired outcome.

Within such a framework preferred embodiments of the disclosed embodiments are directed to a method of treating a subject suffering from neoplastic disorder comprising the step of administering a therapeutically effective amount of at least one neutralizing Notum modulator.

Other embodiments are directed to a method of treating a subject suffering from a Notum associated disorder comprising the step of administering a therapeutically effective amount of at least one depleting Notum modulator.

In yet another embodiment the present invention provides methods of maintenance therapy wherein the disclosed effectors are administered over a period of time following an initial procedure (e.g., chemotherapeutic, radiation or surgery) designed to remove at least a portion of the tumor mass. Such therapeutic regimens may be administered over a period of weeks, a period of months or even a period of years wherein the Notum modulators may act prophylactically to inhibit metastasis and/or tumor recurrence. In yet other embodiments the disclosed modulators may be administrated in concert with known debulking regimens to prevent or retard metastasis.

Beyond the therapeutic uses discussed above it will also be appreciated that the modulators of the instant invention may be used to diagnose Notum related disorders and, in particular, hyperproliferative disorders. As such, a preferred embodiment comprises a method of diagnosing a hyperproliferative disorder in a subject in need thereof comprising the steps of:

a. obtaining a tissue sample from said subject;

b. contacting the tissue sample with at least one Notum modulator; and

c. detecting or quantifying the Notum modulator associated with the sample.

Such methods may be easily discerned in conjunction with the instant application and may be readily performed using generally available commercial technology such as automatic plate readers, dedicated reporter systems, etc. In preferred embodiments the detecting or quantifying step will comprise a reduction of tumor initiating cell frequency. Moreover, limiting dilution analysis may be conducted as previously alluded to above and will preferably employ the use of Poisson distribution statistics to provide an accurate accounting as to the reduction of frequency.

In a similar vein the present invention also provides kits that are useful in the diagnosis and monitoring of Notum associated disorders such as cancer. To this end the present invention preferably provides an article of manufacture useful for diagnosing or treating Notum associated disorders comprising a receptacle comprising a Notum modulator and instructional materials for using said Notum modulator to treat or diagnose the Notum associated disorder.

Other preferred embodiments of the invention also exploit the properties of the disclosed modulators as an instrument useful for identifying, isolating, sectioning or enriching populations or subpopulations of tumor initiating cells through methods such as fluorescence activated cell sorting (FACS) or laser mediated sectioning.

As such, another preferred embodiment of the instant invention is directed to a method of identifying, isolating, sectioning or enriching a population of tumor initiating cells comprising the step of contacting said tumor initiating cells with a Notum modulator.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIGS. 1A-D depict, respectively, the nucleic acid sequence encoding human Notum (SEQ ID NO: 1), the corresponding amino acid sequence of the human Notum precursor protein comprising an amino terminus signal sequence (SEQ ID NO: 2), an alignment of partial macaque, murine and human protein Notum sequences showing amino acid differences (SEQ ID NOS: 99-102) and the amino acid (SEQ ID NO: 333) and nucleic acid (SEQ ID NO: 334) sequence of an exemplary Notum modulator in the form of a Fc-Notum fusion construct wherein the Notum portion is underlined;

FIG. 2 is a graphical representation depicting the gene expression levels of human Notum obtained using whole transcriptome sequencing;

FIG. 3 is a graphical representation showing the relative gene expression levels of human Notum in highly enriched tumor progenitor cell (TProg) and tumor perpetuating cell (TPC) populations obtained from untreated and irinotecan treated mice bearing one of three different non-traditional xenograft (NTX) colorectal tumor cell lines, and normalized against non-tumorigenic (NTG) enriched cell populations as measured using quantitative RT-PCR;

FIGS. 4A and 4B are graphical representations showing the relative gene expression levels of human Notum in whole colorectal tumor specimens from patients with Stage I-IV disease, as normalized against the mean of expression in normal colon and rectum tissue;

FIGS. 5A and 5B are graphical representations showing the relative or absolute gene expression levels, respectively, of human Notum in whole tumor specimens (grey box) or matched NAT (white box) from patients with one of eighteen different solid tumor types;

FIG. 6 is a graphical representation showing the relative expression of human Notum protein in normal adjacent (white) or tumor (black) tissue from specimens obtained from patients with one of eleven different tumor types along with 293T control cells without (white) or without (black) overexpression of p53;

FIGS. 7A and 7B are tabular representations showing, respectively, the genetic arrangement and the heavy and light chain CDR sequences as defined by Chothia et al. of thirty-eight discrete Notum modulators isolated and cloned as described in the Examples herein;

FIGS. 8A-X provide the nucleic acid and amino acid sequences of the heavy and light chain variable regions of twenty-four discrete anti-Notum antibodies isolated and cloned as described in the Examples herein;

FIGS. 9A-D are graphical representations of a canonical Wnt3A assay and the effects of the soluble Notum modulators Notum-hFc and Notum-His (human, mouse and macaque) along with the mutant Notum construct S232A as measured by the same;

FIG. 10 graphically illustrates the activities of several anti-Notum antibodies with respect to the inhibition of active Notum as measured using a canonical Wnt3A assay as normalized against uninhibited Wnt-induced luciferase activity;

FIGS. 11A-D are graphical representations of a canonical Wnt3A assay as used to measure the effects of Notum modulators SC2.D2.2 and SC2.A106 (aka 10B3) on soluble Notum constructs Notum-His and Notum-hFc at various concentrations as normalized against uninhibited Wnt-induced luciferase activity;

FIGS. 12A and 12B graphically illustrate a species specific lack of activity by Notum modulators SC2.D2.2 and SC2.A106 (aka 10B3) using a canonical Wnt3A assay wherein neither modulator exhibits appreciable inhibition of macaque or murine soluble Notum construct antagonism of the Wnt pathway;

FIGS. 13A and 13B provide data establishing an effective co-culture Wnt3A assay that illustrates the effects of endogenously expressed Notum in mixed cell populations (FIG. 13A) and the influence of Notum modulator SC2.D2.2 on the same (FIG. 13B);

FIGS. 14A and 14B are representations of Western Blots showing that both polyclonal antibodies directed to Notum and monoclonal antibody Notum modulators of the instant invention detect Notum in selected protein cell lysates;

FIGS. 15A-G are graphical representations of Notum protein levels from individual patient cell lysate samples as measured using Notum modulator SC2.A109 showing Notum upregulation in several different tumor types and at different stages of diseases;

FIGS. 16A-C illustrate the ability of hNotum proteins (His and hFc) to increase colorectal tumor cell proliferation and/or resistance to apoptosis in a cell based assay and the ability of Notum modulators to antagonize such Notum mediated effects;

FIGS. 17A-C are graphical representations of various aspects of a biochemical assay quantifying the esterase activity of mouse, macaque and human Notum along with an inoperative mutant thereof using two different chromogenic esterase substrates (p-nitrophenyl acetate (PNPA) and p-nitrophenyl butyrate (PNPB));

FIGS. 18A and 18B illustrate the ability of the disclosed Notum modulators to inhibit the esterase activity of hNotum in vitro where the concentration of hNotum is varied in FIG. 18A and the concentration of the Notum modulator is varied in FIG. 18B;

FIG. 19 is a graphical representation of a biochemical assay quantifying the lipase activity of hNotum (gray bars) as presented with a positive control of porcine pancreatic lipase (black bars);

FIG. 20 graphically illustrates the ability of the disclosed Notum modulators to inhibit the lipase activity of hNotum in vitro where the concentration of hNotum is held constant and the concentration of the Notum modulator is varied;

FIGS. 21A and 21B graphically illustrate the inability of point mutated human Notum (S232A and D340A) to antagonize the activity of Wnt3A in 293.TCF cells using a TCF reporter assay (FIG. 21A) and a 4MUH assay (FIG. 21B);

FIG. 22 is a simplified diagram of the canonical Wnt signaling pathway depicting the activation of LEF/TCF transcription factors;

FIG. 23 illustrates the ability of the disclosed Notum modulators to antagonize Notum mediated Wnt3A activity as demonstrated by the activation of luciferase transcription in 293.TCF cells wherein LiCl acts as a positive control;

FIGS. 24A and 24B are graphical representations displaying the ability of the disclosed Notum modulators to antagonize the ability of a chimeric Notum protein to inhibit Wnt3A activity protein levels where FIG. 24A demonstrates that the chimeric Notum can inhibit Wnt3A activity and FIG. 24B shows that the addition of Notum modulators can restore the activity;

FIGS. 25A and 25B illustrate that point mutated Notum constructs retain their ability to interfere with Wnt3A induction of luciferase activity in both a TCF assay (FIG. 25A) and 4MUH assay (FIG. 25B);

FIGS. 26A and 26B are graphical representations demonstrating that certain point mutations made in human and macaque Notum can interfere with the ability of Notum modulator SC2.D2.2 to antagonize Notum enzymatic activity as measured in a TCF assay (FIG. 26A) and 4MUH assay (FIG. 26B);

FIGS. 27A and 27B are graphical representations of illustrating the ability of the disclosed Notum modulators to inhibit Notum mediated antagonism of Wnt3A activity in a TCF assay when the Notum modulator is incubated with Notum and exposed to the cells before the addition of Wnt3A CM (FIG. 27A) and preincubated with Wnt3A CM before exposure to the cells (FIG. 27B);

FIGS. 28A and 28B demonstrate the ability of a small molecule in the form of orlistat to function as a Notum modulator and inhibit the hydrolytic activity of Notum on 4MUH in a dose dependent manner as measured at 4MUH concentrations of 240 μM (FIG. 28A) and 90 μM (FIG. 28B);

FIGS. 29A and 29B are Western blots representing the partitioning of Wnt3A upon in vitro delipidation by Notum (FIG. 29A) and the ability of Notum modulators to inhibit the same (FIG. 29B);

FIG. 30 graphically illustrates the enzymatic neutralizing properties of the disclosed Notum modulators on macaque, mouse and human Notum as measured using a TCF assay;

FIGS. 31A and 31B respectively illustrate the aligned amino acid sequences of the heavy and light chain variable regions of SC2.D2.2 (SEQ ID NO: 56 and SEQ ID NO: 58) and humanized SC2.D2.2 (SEQ ID NO: 331 and SEQ ID NO: 332) wherein the top sequence is the humanized derivative and the vertical marks indicate the respective amino acids are the same and wherein the CDR sequences as defined by Chothia et al. are underlined;

FIGS. 32A-C graphically represent the measured affinity of murine SC2.D2.2 vs. five different concentrations of antigen, and compares the affinity of murine SC2.D2.2 and humanized SC2.D2.2 respectively as determined using label free interaction analysis with a fixed amount of antibody and serial dilutions of antigen; and

FIGS. 33A and 33B illustrate, respectively, a standard curve generated using the disclosed modulators and the plasma concentration of Notum as measured in samples obtained from healthy subjects and patients suffering from ovarian cancer and extrapolated from the standard curve.

### DETAILED DESCRIPTION OF THE INVENTION

I. Introduction

In a broad sense, embodiments of the present invention are directed to novel Notum modulators and their use in treating, managing, ameliorating or preventing the occurrence of hyperproliferative disorders including cancer. Without wishing to be bound by any particular theory, it has been discovered that the disclosed modulators are effective in reducing or retarding tumor growth and eliminating or neutralizing tumorigenic cells as well as altering the sensitivity of such cells to anti-cancer agents. Further, it has surprisingly been discovered that there is a heretofore unknown phenotypic association between selected tumor perpetuating cells (TPC) and the protein known as Notum. In this regard it has been found that selected TPC (i.e., cancer stem cells or CSC), express elevated levels of Notum when compared to normal tissue as well as when compared to tumor progenitor cells (TProg), and non-tumorigenic (NTG) cells that together comprise much of a solid tumor. Thus, in selected embodiments Notum comprises a tumor associated marker (or antigen) and has been found to provide an effective agent for the detection, sensitization and/or suppression of TPC and related neoplasia due to elevated levels of the protein associated with the surface of selected cells and in the tumor microenvironment. More specifically, and even more surprisingly given that Notum is apparently secreted (at least to some extent), it has further been discovered that Notum modulators, including Fc-Notum constructs and immunoreactive antagonists (e.g., antibodies to the protein), may be useful in depleting, sensitizing, eliminating, reducing, reprogramming, promoting the differentiation of, or otherwise precluding or limiting the ability of these tumor perpetuating cells to spread and/or continue to fuel tumor growth or recurrence in a patient.

In preferred embodiments the Notum modulators of the present invention will comprise nucleotides, oligonucleotides, polynucleotides, peptides or polypeptides. As previously alluded to and discussed in detail below, selected embodiments disclosed herein will comprise antibodies to Notum in conjugated or unconjugated forms. Other embodiments of the Notum modulators will preferably comprise Notum or a form, variant, derivative or fragment thereof including, for example, Notum fusion constructs (e.g., Notum-Fc, Notum-targeting moiety, etc.) or Notum-conjugates (e.g., Notum-PEG, Notum-cytotoxic agent, etc.). In yet other embodiments the modulators may operate on the genetic level and may comprise compounds as antisense constructs, siRNA, miRNA and the like. The foregoing Notum modulators may attenuate the growth, propagation or survival of tumor perpetuating cells and/or associated neoplasia through competitive mechanisms, agonizing or antagonizing selected pathways or eliminating or depleting specific cells (including non-TPC support cells) depending, for example, on the form of Notum modulator or dosing and method of delivery.

In view of these discoveries those skilled in the art will appreciate that particularly preferred embodiments of the invention are largely directed to Notum modulators and their use in reducing the frequency of tumor initiating cells. As will be discussed extensively herein, Notum modulators compatible with instant invention broadly comprise any compound that associates, binds, complexes or otherwise reacts or competes with Notum and, optionally, provides for a reduction in tumor perpetuating cell frequency. Exemplary modulators disclosed herein comprise nucleotides, oligonucleotides, polynucleotides, peptides or polypeptides. In certain preferred embodiments the selected modulators will comprise antibodies to Notum or immunoreactive fragments or derivatives thereof. Such antibodies may be antagonistic or agonistic in nature. In other preferred embodiments effectors compatible with the instant invention will comprise Notum constructs comprising Notum itself or a reactive fragment thereof. It will be appreciated that such Notum constructs may comprise fusion proteins and can include reactive domains from other polypeptides such as immunoglobulins, stapled peptides or biological response modifiers. In still other preferred aspects the Notum effector or modulator will comprise a nucleic acid assembly that exerts the desired effects at a genomic level. Still other modulators compatible with the instant teachings will be discussed in detail below.

In a related note, the following discussion pertains to Notum modulators, Notum antagonists and anti-Notum antibodies. While a more detailed definition of each term is provided below, it will be appreciated that the terms are largely interchangeable for the purposes of this disclosure and should not be construed narrowly unless dictated by the context. For example, if a point is made relating to Notum antagonists it is also applicable to those antibodies of the instant invention that happen to be antagonistic. Similarly, the term Notum modulators expressly include disclosed Notum antagonists and anti-Notum antibodies and references to the latter are also applicable to modulators to the extent not precluded by context.

II. Notum

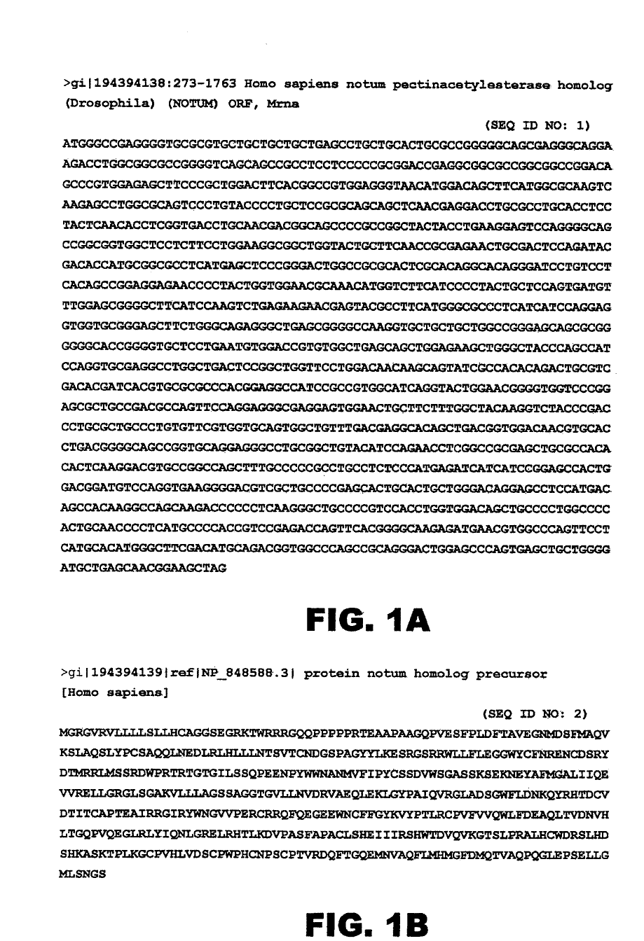

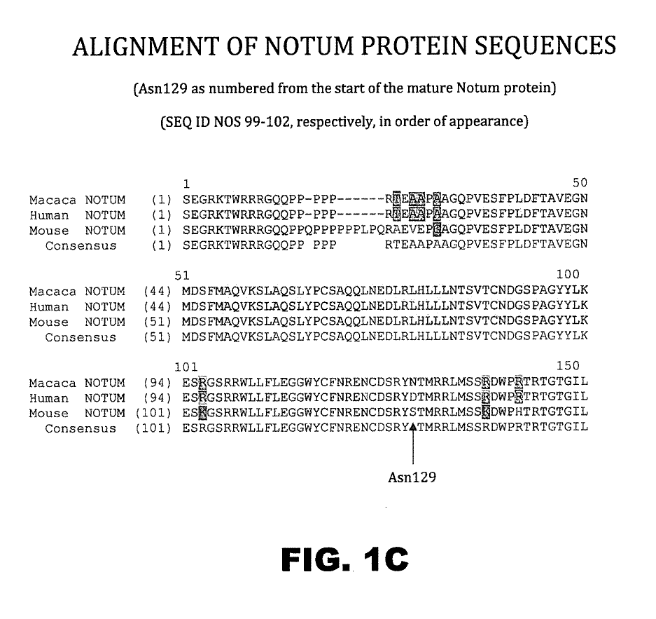

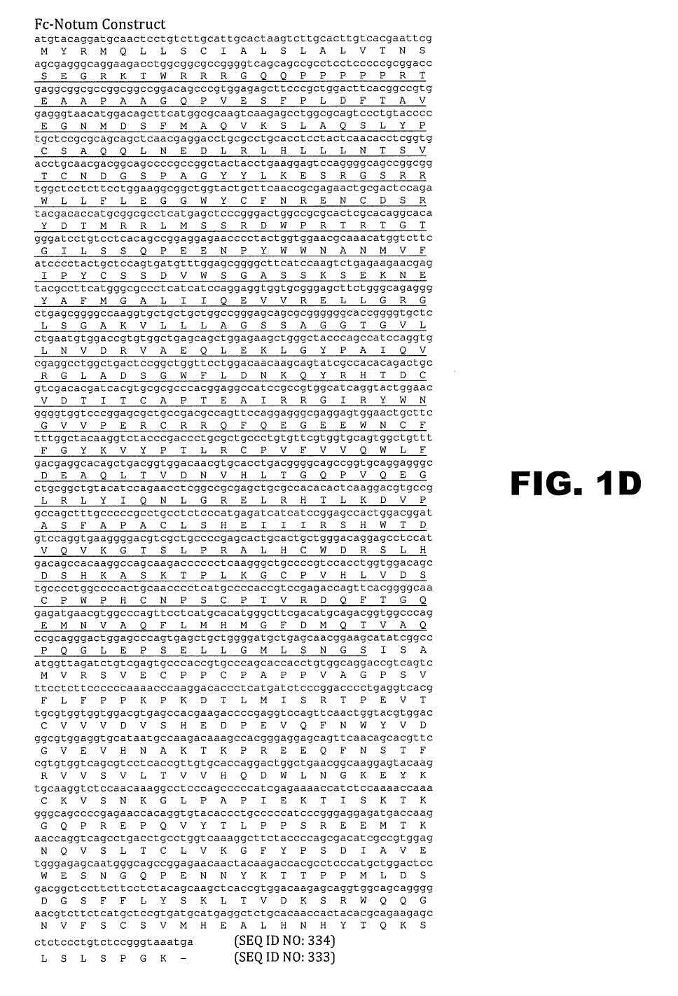

As used herein the term Notum refers to naturally occurring Notum pectinacetylesterase protein, fragments, or variants thereof. Representative Notum orthologs include, but are not limited to, human (i.e. hNotum), mouse, macaque monkey and drosophila. The human ortholog of the gene comprises a 1488 base pair open reading frame which provides for a 496 amino acid (aa) polypeptide construct having a molecular weight of approximately 55.7 kDa. An exemplary nucleic acid sequence encoding human Notum protein is shown in SEQ ID NO: 1 while the corresponding amino acid sequence is shown in SEQ ID NO: 2 (FIGS. 1A and 1B respectively). It will be appreciated that the human Notum protein includes a predicted signal or leader sequence comprising amino acids 1-19 of SEQ ID NO: 2 which is clipped off to provide the mature form of the protein (i.e. 477 aa). By way of reference, murine Notum (GenBank Accession No.: NM_{—}175263) is approximately 91% homologous with human Notum while macaque Notum (GenBank Accession No.: XM_{—}001112829) is approximately 96% homologous. Unless otherwise indicated by direct reference or contextual necessity the term Notum shall be directed to human Notum and immunoreactive equivalents. The human homolog of Notum (GenBank Accession No.: NM_{—}178493; GenelD 147111) is more fully described in Torisu et al. 2008, PMID: 18429952 which is incorporated herein by reference. It will further be appreciated that the term may also refer to a fragment of a native or variant form of Notum that contains an epitope to which an antibody can specifically bind.

Again, while not wishing to be bound by any particular theory, it is believed that Notum modulators, and particularly Notum antagonists, of the present invention may act, at least in part, by interfering with oncogenic survival outside the context of standard of care therapeutic regimens (e.g. irinotecan), as well as reducing or eliminating tumor initiating cell signaling. For example, elimination of TPC by antagonizing Notum may include simply promoting cell proliferation in the face of chemotherapeutic regimens that eliminate proliferating cells, or promote differentiation of TPC such that their self-renewal (i.e., unlimited proliferation) capacity is lost.

As previously indicated, Notum appears to be particularly involved in the Wnt, Hh and BMP pathways. In this respect those skilled in the art will appreciate that Notum is a secreted hydrolase initially identified in Drosophila as repressing Wingless (Wg) activity by modifying the heparin sulfate proteoglycans Dally-like (Dlp) and Dally. In Drosophila the Notum gene appears to encode a protein of 671 amino acid residues, which is related to plant pectin acetylesterases of the α/β hydrolase superfamily. More recent evidence has demonstrated that drosophila Notum (dNotum) can also function as a lipase, releasing Dlp from the cell surface by cleaving Dlp's glycosylphosphatidylinositol (GPI) anchor. Modifications and/or release of these cell surface proteoglycans by Notum results in a sharp reduction in the cell surface levels of Dally protein expression and the conversion of Dlp into a modified form as evidenced by gel electrophoresis. Such observations indicate that dNotum antagonizes Wg and Hedgehog (Hh) signaling augmented by Dally and Dlp, most likely by modifying their glycoaminoglycan side chains and/or releasing Dlp from the cell surface. These modifications by dNotum act to modify localized Wg and Hedgehog concentrations and thus antagonize interactions of these morphogens with their receptors. Moreover, release of Wg or Hedgehog proteins associated with Dally or Dlp from the cell surface promotes long-range activity of these morphogens, having major impacts on tissue patterning during development. See generally: Ayers et al., 2010, PMID: 20412775; Giraldez et al., 2002, PMID: 12015973 and Traister et al., 2008, PMID: 17967162; each of which is incorporated herein in its entirety by reference.

Various studies have also shown that Dally and Dlp-related proteoglycans likely play important roles in Wnt signaling in vertebrates (Topczewski et al. 2001, PMID: 11702784 and Filmus et al., 2008, PMID: 18505598), and that Notum acts to modulate Wnt signaling via its receptor Frizzled, much as the analogous protein does in Drosophila. As with Wg, mammalian Notum is proposed to downregulate the Wnt pathway by releasing glycosyl-phosphatidylinositol-anchored (GPI) glypicans (analogous to Dlp and Dally) from the cell surface. (Traister et al., supra). When bound to the cell surface, GPI-anchored glypicans promote Wnt signaling by stabilizing the interaction of various forms of Wnt with their Frizzled receptors, whereas glypicans that have been released from the cell surface repress Wnt signaling by competitively inhibiting Wnt interactions with GPI-anchored, cell surface glypicans that are proximal to Frizzled receptors (Filmus et al., supra). The absence, or decreased local concentration, of glypicans at the very least increases the threshold of Wnt concentrations that must be present at the cell surface to stimulate beta-catenin pathway signaling via Fzd receptors. These data, along with additional studies have shown that mammalian (e.g. human) Notum antagonizes Wnt signaling. Notum has also been identified as a Wnt/beta-catenin target for transcriptional activation, suggesting that Notum is a feedback inhibitor of the Wnt/Fzd/beta-catenin signaling cascade.

Wnt/Fzd signaling plays a large role in cell fate determination decisions within many tissues during organogenesis and development, and perturbation of these pathways often results in cancer. Moreover, multiple mouse genetic models wherein stem cells of the lower gastrointestinal tract have been identified and/or manipulated show that signaling via the Wnt/beta-catenin pathway impact tissue-resident stem cell differentiation decisions leading to the generation of Paneth cells, which themselves have been suggested to support stem cell self-renewal and expansion at the base of tissue structures known as crypts; which is where the stem cells are known to reside. Deregulation of Wnt signaling by Notum and/or impaired feedback regulation of this pathway by increased localized concentrations of Notum proximal to the TPC population may contribute to tumorigenesis, continued tumor growth and tumor recurrence. Modifying this contribution with Notum modulators may have therapeutic benefit by altering Wnt gradient formation proximal to the cell surface of tumor cells.

Given Notum's ability to effectively reduce glypican concentrations at the cell surface, Notum is also likely to exert control over Hedgehog (Hh) morphogen gradients by releasing glypicans from cell surface. As noted above in Drosophila, Dally and Dlp-related glypicans can also bind Hh to actively compete with the Hh receptor, Patched (Ptc). Competition with Ptc for Hh binding effectively reduces proximal Hh binding to Ptc, resulting in decreased signaling through Smoothened, which acts on Hh effector pathways via the Gli-family of transcription factors. By cleaving glypican from the cell surface, Notum reduces the concentration of membrane proximal competition for Hh and thus increases Hh signaling via Smoothened by promoting higher effective concentrations of Hh that bind to and inhibit the Smoothened repressor, Ptc (Traister et al., and Filmus, both supra), potentially replicating genetic models that activate the Hh signaling cascade via genetic inactivation of Ptc. Like Wnt family proteins, Hh proteins are lipid modified and diffuse very little without the help of associated proteins (e.g. glypican) that improve the solubility of the overall complex (Eaton S., 2006, PMID: 16364628).

Hh morphogen gradients are critically important for organogenesis and development of various solid tissues and perturbation of Hh morphogen gradients or the ability to inhibit Smoothened signaling via Ptc is associated with abnormal development and cancer. It should also be recognized that by promoting increased shedding of glypican and its associated Hh proteins, Notum may also create new concentration gradients of Hh that did not previously exist due to the poor solubility characteristics of Hh and its tight association with glypican. While Hh signaling normally acts in concert with other morphogen signaling pathways to control normal cell fate decisions, constitutive activation of Smoothened has been shown to result in basal cell carcinomas, medullablastoma and pancreatic neoplasms. There is also much evidence that elevated Hh signaling can cooperate with APC and/or KRAS lesions, for example, to amplify cancer onset and severity. Elevated Notum levels proximal to TPC may be a critical and as yet unrecognized player in oncogenesis and tumor progression due to the ability of Notum to promote increased local concentrations of Hh and, prospectively, new distal concentration gradients of glypican-associated Hh.

Finally, glypicans have been shown to regulate local concentration gradients of BMP/TGF-beta family members in a variety of tissues (Paine-Saunders et al., 2000, PMID 10964473) and thus the sensitivity of glypicans to Notum cleavage and release from the cell surface could in point of fact promote cancer progression as is observed in tumors and murine cancer models where BMP receptor signaling is decreased and/or functionally inactivated (Kodach et al., 2008, PMID: 18008360 and Hardwick et al., 2008, PMID: 18756288). By way of example, BMP receptor mutations are occasional contributors to juvenile polyposis syndrome and cancer in humans.

As discussed above, glypicans regulate different kinds of growth factors and morphogens in a tissue-specific manner. Altered gene expression of glypicans, independent of Notum expression, has also been shown to mediate oncogenesis. Glypican-3, for example, inhibits proliferation and induces cell death in certain tumor types. As such, Glypican-3 acts as a tumor suppressor and is downregulated in a number of tumors of different origin (Filmus 2001, PMID: 11320054). In the framework of the instant invention it is believed that, in tumors wherein TPC are expressing elevated levels of Notum, glypican concentrations are effectively reduced and these reductions contribute to oncogenesis and tumor progression. As disclosed herein, the provided Notum modulators can attenuate these levels and likely impart the desired anti-neoplastic response.

In addition to the aforementioned glypican mediated regulation, the lipase activity of Notum (as exemplified in Example 24 below) suggests additional mechanisms whereby it may modulate Wnt activity; e.g., delipidation of Wnt proteins may modulate their interactions with chaperones, affecting longer range transport of Wnts, as well as perturbing interactions with Wnt receptors and co-receptors. A broad based lipase activity may also perturb other signaling pathways mediated by lipid modified proteins (e.g. BMP, Wnt & Hh). As such, the Notum modulators disclosed herein may interfere with this enzymatic activity to further reduce the frequency of tumor initiating cells and inhibit neoplastic growth and/or metastasis.

Although these pathways have been extensively studied in the past few years, the role of Notum has not been fully recognized or exploited prior to the elucidation of the present invention. In this respect, gene expression profiling of various solid tumors including hepatocellular, gastric, colorectal and pancreatic cancer has shown Notum to be overexpressed in patients with these neoplasms. See e.g., U.S. Ser. No. 10/568,471, U.S. Ser. No. 10/301,822, U.S. Pat. No. 7,371,840 and Torisu et al., supra; each of which is incorporated herein by reference in its entirety. While production of a single antibody to human Notum was demonstrated in U.S. Ser. No. 10/568,471, there was no evidence presented that such an antibody would be effective in any type of a therapeutic setting. Moreover, unlike the novel Notum modulators of the present invention, there was absolutely no indication that the disclosed antibody could antagonize secreted Notum to produce the anti-neoplastic effects disclosed herein. Nor is there any indication in any of the references that Notum is associated with tumor initiating cells, or that this association affords an effective mechanism by which these tumor instigators may be sensitized, eliminated or otherwise neutralized, thereby allowing for efficacious treatment of the heterogeneous tumor bulk.

III. Tumor Initiating Cells

In contrast to any teachings of the prior art, the present invention provides Notum modulators that are particularly useful for targeting tumor initiating cells, and especially tumor perpetuating cells, thereby facilitating the treatment, management or prevention of neoplastic disorders. More specifically, as previously indicated it has surprisingly been found that specific tumor cell subpopulations express Notum and likely modify localized coordination of morphogen signaling important to cancer stem cell self-renewal and cell survival. Thus, in preferred embodiments modulators of Notum may be used to reduce tumor initiating cell frequency in accordance with the present teachings and thereby facilitate the treatment or management of hyperproliferative diseases.

As used herein, the term tumor initiating cell (TIC) encompasses both tumor perpetuating cells (TPC; i.e., cancer stem cells or CSC) and highly proliferative tumor progenitor cells (termed TProg), which together generally comprise a unique subpopulation (i.e. 0.1-40%) of a bulk tumor or mass. For the purposes of the instant disclosure the terms tumor perpetuating cells and cancer stem cells are equivalent and may be used interchangeably herein. Conversely, TPC differ from TProg in that they can completely recapitulate the composition of tumor cells existing within a tumor and have unlimited self-renewal capacity as demonstrated by serial transplantation (two or more passages through mice) of low numbers of isolated cells. As will be discussed in more detail below fluorescence-activated cell sorting (FACS) using appropriate cell surface markers is a reliable method to isolate highly enriched cell subpopulations (>99.5% purity) due, at least in part, to its ability to discriminate between single cells and clumps of cells (i.e. doublets, etc.). Using such techniques it has been shown that when low cell numbers of highly purified TProg cells are transplanted into immunocompromised mice they can fuel tumor growth in a primary transplant. However, unlike purified TPC subpopulations the TProg generated tumors do not completely reflect the parental tumor in phenotypic cell heterogeneity and are demonstrably inefficient at reinitiating serial tumorigenesis in subsequent transplants. In contrast, TPC subpopulations completely reconstitute the cellular heterogeneity of parental tumors and can efficiently initiate tumors when serially isolated and transplanted. Thus, those skilled in the art will recognize that a definitive difference between TPC and TProg, though both may be tumor generating in primary transplants, is the unique ability of TPC to perpetually fuel heterogeneous tumor growth upon serial transplantation at low cell numbers. Other common approaches to characterize TPC involve morphology and examination of cell surface markers, transcriptional profile, and drug response although marker expression may change with culture conditions and with cell line passage in vitro.

Accordingly, for the purposes of the instant invention tumor perpetuating cells, like normal stem cells that support cellular hierarchies in normal tissue, are preferably defined by their ability to self-renew indefinitely while maintaining the capacity for multilineage differentiation. Tumor perpetuating cells are thus capable of generating both tumorigenic progeny (i.e., tumor initiating cells: TPC and TProg) and non-tumorigenic (NTG) progeny. As used herein a non-tumorigenic cell (NTG) refers to a tumor cell that arises from tumor initiating cells, but does not itself have the capacity to self-renew or generate the heterogeneous lineages of tumor cells that comprise a tumor. Experimentally, NTG cells are incapable of reproducibly forming tumors in mice, even when transplanted in excess cell numbers.

As indicated, TProg are also categorized as tumor initiating cells (or TIC) due to their limited ability to generate tumors in mice. TProg are progeny of TPC and are typically capable of a finite number of non-self-renewing cell divisions. Moreover, TProg cells may further be divided into early tumor progenitor cells (ETP) and late tumor progenitor cells (LTP), each of which may be distinguished by phenotype (e.g., cell surface markers) and different capacities to recapitulate tumor cell architecture. In spite of such technical differences, both ETP and LTP differ functionally from TPC in that they are generally less capable of serially reconstituting tumors when transplanted at low cell numbers and typically do not reflect the heterogeneity of the parental tumor. Notwithstanding the foregoing distinctions, it has also been shown that various TProg populations can, on rare occasion, gain self-renewal capabilities normally attributed to stem cells and themselves become TPC (or CSC). In any event both types of tumor-initiating cells are likely represented in the typical tumor mass of a single patient and are subject to treatment with the modulators as disclosed herein. That is, the disclosed compositions are generally effective in reducing the frequency or altering the chemosensitivity of such Notum positive tumor initiating cells regardless of the particular embodiment or mix represented in a tumor.

In the context of the instant invention, TPC are more tumorigenic, relatively more quiescent and often more chemoresistant than the TProg (both ETP and LTP), NTG cells and the tumor-infiltrating non-TPC derived cells (e.g., fibroblasts/stroma, endothelial & hematopoietic cells) that comprise the bulk of a tumor. Given that conventional therapies and regimens have, in large part, been designed to both debulk tumors and attack rapidly proliferating cells, TPC are likely to be more resistant to conventional therapies and regimens than the faster proliferating TProg and other bulk tumor cell populations. Further, TPC often express other characteristics that make them relatively chemoresistant to conventional therapies, such as increased expression of multi-drug resistance transporters, enhanced DNA repair mechanisms and anti-apoptotic proteins. These properties, each of which contribute to drug tolerance by TPC, constitute a key reason for the failure of standard oncology treatment regimens to ensure long-term benefit for most patients with advanced stage neoplasia; i.e. the failure to adequately target and eradicate those cells that fuel continued tumor growth and recurrence (i.e. TPC or CSC).

Unlike many of the aforementioned prior art treatments, the novel compositions of the present invention preferably reduce the frequency of tumor initiating cells upon administration to a subject regardless of the form or specific target (e.g., genetic material, Notum or Notum ligand) of the selected modulator. As noted above, the reduction in tumor initiating cell frequency may occur as a result of a) elimination, depletion, sensitization, silencing or inhibition of tumor initiating cells; b) controlling the growth, expansion or recurrence of tumor initiating cells; c) interrupting the initiation, propagation, maintenance, or proliferation of tumor initiating cells; or d) by otherwise hindering the survival, regeneration and/or metastasis of the tumorigenic cells. In some embodiments, the reduction in the frequency of tumor initiating cells occurs as a result of a change in one or more physiological pathways. The change in the pathway, whether by reduction or elimination of the tumor initiating cells or by modifying their potential (e.g., induced differentiation, niche disruption) or otherwise interfering with their ability to exert affects on the tumor environment or other cells, in turn allows for the more effective treatment of Notum-associated disorders by inhibiting tumorigenesis, tumor maintenance and/or metastasis and recurrence.

Among the methods that can be used to assess such a reduction in the frequency of tumor initiating cells is limiting dilution analysis either in vitro or in vivo, preferably followed by enumeration using Poisson distribution statistics or assessing the frequency of predefined definitive events such as the ability to generate tumors in vivo or not. While such limiting dilution analysis are the preferred methods of calculating reduction of tumor initiating cell frequency, other, less demanding methods, may also be used to effectively determine the desired values, albeit slightly less accurately, and are entirely compatible with the teachings herein. Thus, as will be appreciated by those skilled in the art, it is also possible to determine reduction of frequency values through well-known flow cytometric or immunohistochemical means. As to all the aforementioned methods see, for example, Dylla et al. 2008, PMCID: PMC2413402 & Hoey et al. 2009, PMID: 19664991; each of which is incorporated herein by reference in its entirety.

With respect to limiting dilution analysis, in vitro enumeration of tumor initiating cell frequency may be accomplished by depositing either fractionated or unfractionated human tumor cells (e.g. from treated and untreated tumors, respectively) into in vitro growth conditions that foster colony formation. In this manner, colony forming cells might be enumerated by simple counting and characterization of colonies, or by analysis consisting of, for example, the deposition of human tumor cells into plates in serial dilutions and scoring each well as either positive or negative for colony formation at least 10 days after plating. In vivo limiting dilution experiments or analyses, which are generally more accurate in their ability to determine tumor initiating cell frequency encompass the transplantation of human tumor cells, from either untreated control or treated conditions, for example, into immunocompromised mice in serial dilutions and subsequently scoring each mouse as either positive or negative for tumor formation at least 60 days after transplant. The derivation of cell frequency values by limiting dilution analysis in vitro or in vivo is preferably done by applying Poisson distribution statistics to the known frequency of positive and negative events, thereby providing a frequency for events fulfilling the definition of a positive event; in this case, colony or tumor formation, respectively.

As to other methods compatible with the instant invention that may be used to calculate tumor initiating cell frequency, the most common comprise quantifiable flow cytometric techniques and immunohistochemical staining procedures. Though not as precise as the limiting dilution analysis techniques described immediately above, these procedures are much less labor intensive and provide reasonable values in a relatively short time frame. Thus, it will be appreciated that a skilled artisan may use flow cytometric cell surface marker profile determination employing one or more antibodies or reagents that bind art recognized cell surface proteins known to enrich for tumor initiating cells (e.g., potentially compatible markers are set forth in Example 1 below) and thereby measure TIC levels from various samples. In still another compatible method one skilled in the art might enumerate TIC frequency in situ (i.e. tissue section) by immunohistochemistry using one or more antibodies or reagents that are able to bind cell surface proteins thought to demarcate these cells.

Using any of the above-referenced methods it is then possible to quantify the reduction in frequency of TIC (or the TPC therein) provided by the disclosed Notum modulators in accordance with the teachings herein. In some instances, the compounds of the instant invention may reduce the frequency of TIC (by a variety of mechanisms noted above, including elimination, induced differentiation, niche disruption, silencing, etc.) by 10%, 15%, 20%, 25%, 30% or even by 35%. In other embodiments, the reduction in frequency of TIC may be on the order of 40%, 45%, 50%, 55%, 60% or 65%. In certain embodiments, the disclosed compounds my reduce the frequency of TIC by 70%, 75%, 80%, 85%, 90% or even 95%. Of course it will be appreciated that any reduction of the frequency of the TIC likely results in a corresponding reduction in the tumorigenicity, persistence, recurrence and aggressiveness of the neoplasia.

IV. Notum Modulators

In any event, the present invention is directed to the use of Notum modulators, including Notum antagonists, for the diagnosis, treatment and/or prophylaxis of any one of a number of Notum associated malignancies. The disclosed modulators may be used alone or in conjunction with a wide variety of anti-cancer compounds such as chemotherapeutic or immunotherapeutic agents or biological response modifiers. In other selected embodiments, two or more discrete Notum modulators may be used in combination to provide enhanced anti-neoplastic effects or may be used to fabricate multispecific constructs.

In certain embodiments, the Notum modulators of the present invention will comprise nucleotides, oligonucleotides, polynucleotides, peptides or polypeptides. Even more preferably the modulators will comprise soluble Notum (sNotum) or a form, variant, derivative or fragment thereof including, for example, Notum fusion constructs (e.g., Notum-Fc, Notum-targeting moiety, etc.) or Notum-conjugates (e.g., Notum-PEG, Notum-cytotoxic agent, Notum-brm, etc.). It will also be appreciated that, in other embodiments, the Notum modulators comprise antibodies (e.g., anti-Notum mAbs) or immunoreactive fragments or derivatives thereof. In particularly preferred embodiments the modulators of the instant invention will comprise neutralizing antibodies or derivatives or fragments thereof. In other embodiments the Notum modulators may comprise internalizing antibodies. In still other embodiments the Notum modulators may comprise depleting antibodies. Moreover, as with the aforementioned fusion constructs, these antibody modulators may be conjugated, linked or otherwise associated with selected cytotoxic agents, polymers, biological response modifiers (BRMs) or the like to provide directed immunotherapies with various (and optionally multiple) mechanisms of action. In yet other embodiments the modulators may operate on the genetic level and may comprise compounds as antisense constructs, siRNA, micro RNA and the like.

It will further be appreciated that the disclosed Notum modulators may deplete or eliminate or inhibit growth, propagation or survival of tumor cells, particularly TPC, and/or associated neoplasia through a variety of mechanisms, including agonizing or antagonizing selected pathways or eliminating specific cells depending, for example, on the form of Notum modulator, any associated payload or dosing and method of delivery. Accordingly, while preferred embodiments disclosed herein are directed to the depletion, inhibition or silencing of specific tumor cell subpopulations such as tumor perpetuating cells it must be emphasized that such embodiments are merely illustrative and not limiting in any sense. Rather, as set forth in the appended claims, the present invention is broadly directed to Notum modulators and their use in the treatment, management or prophylaxis of various Notum mediated hyperproliferative disorders irrespective of any particular mechanism or target tumor cell population.

In the same sense disclosed embodiments of the instant invention comprise one or more Notum antagonists. To that end it will be appreciated that Notum antagonists of the instant invention may comprise any ligand, polypeptide, peptide, fusion protein, antibody or immunologically active fragment or derivative thereof that recognizes, reacts, binds, combines, competes, associates or otherwise interacts with the Notum protein or fragment thereof and eliminates, silences, reduces, inhibits, hinders, restrains or controls the growth of tumor initiating cells or other neoplastic cells including bulk tumor or NTG cells. In selected embodiments the Notum modulator comprises a Notum antagonist.

As used herein an antagonist refers to a molecule capable of neutralizing, blocking, inhibiting, abrogating, reducing or interfering with the activities of a particular or specified protein, including the binding of receptors to ligands or the interactions of enzymes with substrates. More generally antagonists of the invention may comprise antibodies and antigen-binding fragments or derivatives thereof, proteins, peptides, glycoproteins, glycopeptides, glycolipids, polysaccharides, oligosaccharides, nucleic acids, antisense constructs, siRNA, miRNA, bioorganic molecules, peptidomimetics, pharmacological agents and their metabolites, transcriptional and translation control sequences, and the like. Antagonists may also include small molecule inhibitors, fusion proteins, receptor molecules and derivatives which bind specifically to the protein thereby sequestering its binding to its substrate target, antagonist variants of the protein, antisense molecules directed to the protein, RNA aptamers, and ribozymes against the protein.

As used herein and applied to two or more molecules or compounds, the term recognizes or specifically recognizes shall be held to mean the reaction, binding, specific binding, combination, association, interaction, connection, linkage, uniting, coalescence, merger or joining, covalently or non-covalently, of the molecules whereby one molecule exerts an effect on the other molecule.

Moreover, as demonstrated in the examples herein, some modulators of human Notum may, in certain cases, cross-react with Notum from a species other than human (e.g., murine). In other cases exemplary modulators may be specific for one or more isoforms of human Notum and will not exhibit cross reactivity with Notum orthologs from other species.

In any event, those skilled in the art will appreciate that the disclosed modulators may be used in a conjugated or unconjugated form. That is, the modulator may be associated with or conjugated to (e.g. covalently or non-covalently) pharmaceutically active compounds, biological response modifiers, cytotoxic or cytostatic agents, diagnostic moieties or biocompatible modifiers. In this respect it will be understood that such conjugates may comprise peptides, polypeptides, proteins, fusion proteins, nucleic acid molecules, small molecules, mimetic agents, synthetic drugs, inorganic molecules, organic molecules and radioisotopes. Moreover, as indicated above the selected conjugate may be covalently or non-covalently linked to the Notum modulator in various molar ratios depending, at least in part, on the method used to effect the conjugation.

V. Antibodies

a. Overview

As previously alluded to particularly preferred embodiments of the instant invention comprise Notum modulators in the form of antibodies. The term antibody herein is used in the broadest sense and specifically covers synthetic antibodies, monoclonal antibodies, oligoclonal or polyclonal antibodies, multiclonal antibodies, recombinantly produced antibodies, intrabodies, multispecific antibodies, bispecific antibodies, monovalent antibodies, multivalent antibodies, human antibodies, humanized antibodies, chimeric antibodies, primatized antibodies, Fab fragments, F(ab′) fragments, single-chain FvFcs (scFvFc), single-chain Fvs (scFv), anti-idiotypic (anti-Id) antibodies and any other immunologically active antibody fragments so long as they exhibit the desired biological activity (i.e., Notum association or binding). In a broader sense, the antibodies of the present invention include immunoglobulin molecules and immunologically active fragments of immunoglobulin molecules, i.e., molecules that contain an antigen binding site, where these fragments may or may not be fused to another immunoglobulin domain including, but not limited to, an Fc region or fragment thereof. Further, as outlined in more detail herein, the terms antibody and antibodies specifically include Fc variants as described below, including full length antibodies and variant Fc-Fusions comprising Fc regions, or fragments thereof, optionally comprising at least one amino acid residue modification and fused to an immunologically active fragment of an immunoglobulin.

As will be discussed in more detail below, the generic term antibodies or immunoglobulin comprises five distinct classes of antibody that can be distinguished biochemically and, depending on the amino acid sequence of the constant domain of their heavy chains, can readily be assigned to the appropriate class. For historical reasons, the major classes of intact antibodies are termed IgA, IgD, IgE, IgG, and IgM. In humans, the IgG and IgA classes may be further divided into recognized subclasses (isotypes), i.e., IgG1, IgG2, IgG3, IgG4, IgA1, and IgA2 depending on structure and certain biochemical properties. It will be appreciated that the IgG isotypes in humans are named in order of their abundance in serum with IgG1 being the most abundant.

While all five classes of antibodies (i.e. IgA, IgD, IgE, IgG, and IgM) and all isotypes (i.e., IgG1, IgG2, IgG3, IgG4, IgA1, and IgA2), as well as variations thereof, are within the scope of the present invention, preferred embodiments comprising the IgG class of immunoglobulin will be discussed in some detail solely for the purposes of illustration. It will be understood that such disclosure is, however, merely demonstrative of exemplary compositions and methods of practicing the present invention and not in any way limiting of the scope of the invention or the claims appended hereto.

In this respect, human IgG immunoglobulins comprise two identical light polypeptide chains of molecular weight approximately 23,000 Daltons, and two identical heavy chains of molecular weight 53,000-70,000 depending on the isotype. Heavy-chain constant domains that correspond to the different classes of antibodies are denoted by the corresponding lower case Greek letter α, δ, ε, γ, and μ, respectively. The light chains of the antibodies from any vertebrate species can be assigned to one of two clearly distinct types, called kappa (κ) and lambda (λ), based on the amino acid sequences of their constant domains. Those skilled in the art will appreciate that the subunit structures and three-dimensional configurations of different classes of immunoglobulins are well known.

The four chains are joined by disulfide bonds in a Y configuration wherein the light chains bracket the heavy chains starting at the mouth of the Y and continuing through the variable region to the dual ends of the Y. Each light chain is linked to a heavy chain by one covalent disulfide bond while two disulfide linkages in the hinge region join the heavy chains. The respective heavy and light chains also have regularly spaced intrachain disulfide bridges the number of which may vary based on the isotype of IgG.

Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. In this regard, it will be appreciated that the variable domains of both the light (V_{L}) and heavy (V_{H}) chain portions determine antigen recognition and specificity. Conversely, the constant domains of the light chain (C_{L}) and the heavy chain (C_{H}1, C_{H}2 or C_{H}3) confer and regulate important biological properties such as secretion, transplacental mobility, circulation half-life, complement binding, and the like. By convention the numbering of the constant region domains increases as they become more distal from the antigen binding site or amino-terminus of the antibody. Thus, the amino or N-terminus of the antibody comprises the variable region and the carboxy or C-terminus comprises the constant region. Thus, the C_{H}3 and C_{L} domains actually comprise the carboxy-terminus of the heavy and light chain, respectively.

The term variable refers to the fact that certain portions of the variable domains differ extensively in sequence among immunoglobulins and these hot spots largely define the binding and specificity characteristics of a particular antibody. These hypervariable sites manifest themselves in three segments, known as complementarity determining regions (CDRs), in both the light-chain and the heavy-chain variable domains respectively. The more highly conserved portions of variable domains flanking the CDRs are termed framework regions (FRs). More specifically, in naturally occurring monomeric IgG antibodies, the six CDRs present on each arm of the antibody are short, non-contiguous sequences of amino acids that are specifically positioned to form the antigen binding site as the antibody assumes its three dimensional configuration in an aqueous environment.

The framework regions comprising the remainder of the heavy and light variable domains show less inter-molecular variability in amino acid sequence. Rather, the framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen-binding site formed by the positioned CDRs defines a surface complementary to the epitope on the immunoreactive antigen (i.e. Notum). This complementary surface promotes the non-covalent binding of the antibody to the immunoreactive antigen epitope. It will be appreciated that the position of CDRs can be readily identified by one of ordinary skill in the art.

As discussed in more detail below all or part of the heavy and light chain variable regions may be recombined or engineered using standard recombinant and expression techniques to provide effective antibodies. That is, the heavy or light chain variable region from a first antibody (or any portion thereof) may be mixed and matched with any selected portion of the heavy or light chain variable region from a second antibody. For example, in one embodiment, the entire light chain variable region comprising the three light chain CDRs of a first antibody may be paired with the entire heavy chain variable region comprising the three heavy chain CDRs of a second antibody to provide an operative antibody. Moreover, in other embodiments, individual heavy and light chain CDRs derived from various antibodies may be mixed and matched to provide the desired antibody having optimized characteristics. Thus, an exemplary antibody may comprise three light chain CDRs from a first antibody, two heavy chain CDRs derived from a second antibody and a third heavy chain CDR from a third antibody.

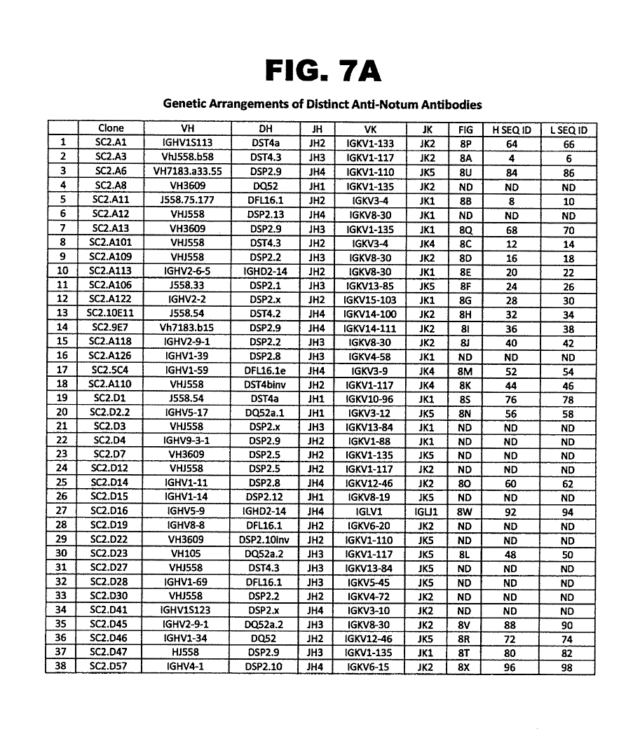

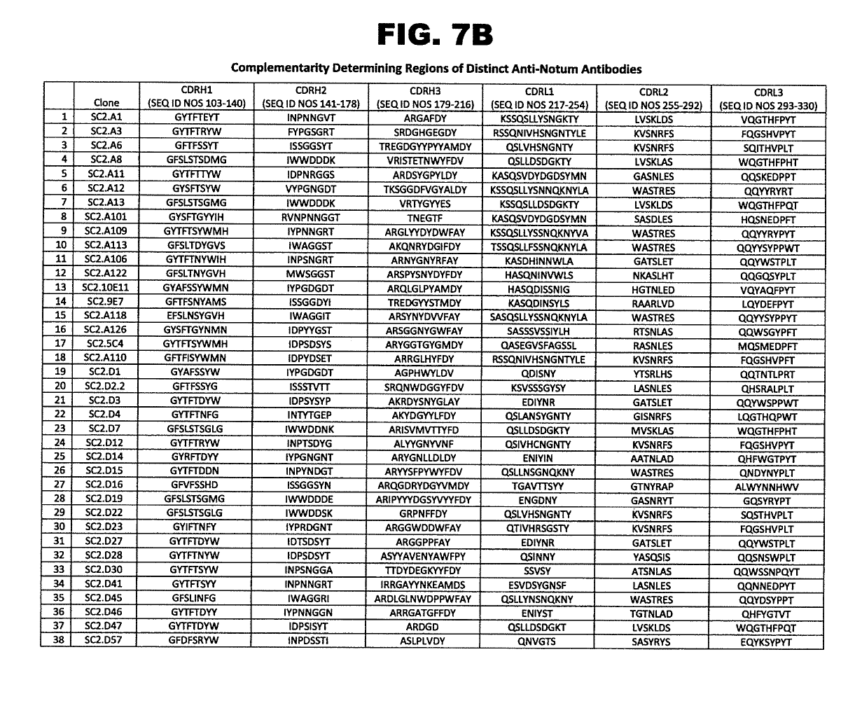

More specifically, in the context of the instant invention it will be appreciated that any of the disclosed heavy and light chain CDRs in FIG. 7B may be rearranged in this manner to provide optimized anti-Notum (e.g. anti-Notum) antibodies in accordance with the instant teachings.

In any event, the complementarity determining regions residue numbers may be defined as those of Kabat et al. (1991, NIH Publication 91-3242, National Technical Information Service, Springfield, Va.), specifically, residues 24-34 (CDR1), 50-56 (CDR2) and 89-97 (CDR3) in the light chain variable domain and 31-35 (CDR1), 50-65 (CDR2) and 95-102 (CDR3) in the heavy chain variable domain. Note that CDRs vary considerably from antibody to antibody (and by definition will not exhibit homology with the Kabat consensus sequences). Maximal alignment of framework residues frequently requires the insertion of spacer residues in the numbering system, to be used for the Fv region. In addition, the identity of certain individual residues at any given Kabat site number may vary from antibody chain to antibody chain due to interspecies or allelic divergence. See also Chothia et al., J. Mol. Biol. 196:901-917 (1987) and by MacCallum et al., J. Mol. Biol. 262:732-745 (1996) where the definitions include overlapping or subsets of amino acid residues when compared against each other. Each of the aforementioned references is incorporated herein by reference in its entirety and the amino acid residues which encompass CDRs as defined by each of the above cited references are set forth for comparison.

CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

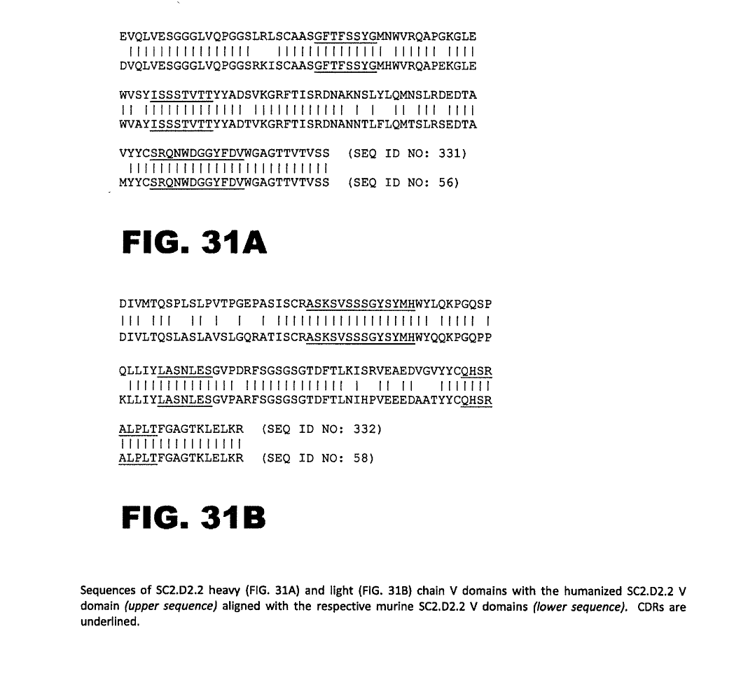

For purposes of convenience the CDRs set forth in FIG. 7B and underlined in FIGS. 31A and 31B are defined using the nomenclature of Chothia et al. though given the content of the instant application one skilled in the art could readily identify and enumerate the CDRs as defined by Kabat et al. or MacCallum et al. for each respective heavy and light chain sequence. Accordingly, antibodies comprising CDRs defined by such nomenclature are expressly included within the scope of the instant invention. More broadly the term variable region CDR amino acid residue includes amino acids in a CDR as identified using any sequence or structure based method as set forth above.

As used herein the term variable region framework (FR) amino acid residues refers to those amino acids in the framework region of an Ig chain. The term framework region or FR region as used herein, includes the amino acid residues that are part of the variable region, but are not part of the CDRs (e.g., using the Kabat definition of CDRs). Therefore, a variable region framework is a non-contiguous sequence between about 100-120 amino acids in length but includes only those amino acids outside of the CDRs.

For the specific example of a heavy chain variable region and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light claim variable region CDRs. Similarly, using the definition of CDRs by Chothia et al. or McCallum et al. the framework region boundaries are separated by the respective CDR termini as described above.

With the aforementioned structural considerations in mind, those skilled in the art will appreciate that the antibodies of the present invention may comprise any one of a number of functional embodiments. In this respect, compatible antibodies may comprise any immunoreactive antibody (as the term is defined herein) that provides the desired physiological response in a subject. While any of the disclosed antibodies may be used in conjunction with the present teachings, certain embodiments of the invention will comprise chimeric, humanized or human monoclonal antibodies or immunoreactive fragments thereof. Yet other embodiments may, for example, comprise homogeneous or heterogeneous multimeric constructs, Fc variants and conjugated or glycosylationally altered antibodies. Moreover, it will be understood that such configurations are not mutually exclusive and that compatible individual antibodies may comprise one or more of the functional aspects disclosed herein. For example, a compatible antibody may comprise a single chain diabody with humanized variable regions or a fully human full length IgG3 antibody with Fc modifications that alter the glycosylation pattern to modulate serum half-life. Other exemplary embodiments are readily apparent to those skilled in the art and may easily be discernable as being within the scope of the invention.

b. Antibody Generation

As is well known various host animals, including rabbits, mice, rats, etc. may be inoculated and used to provide antibodies in accordance with the teachings herein. Art known adjuvants that may be used to increase the immunological response, depending on the inoculated species include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants may protect the antigen from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

After immunization of an animal with a Notum immunogen, antibodies and/or antibody-producing cells can be obtained from the animal using art recognized techniques. In some embodiments, polyclonal anti-Notum antibody-containing serum is obtained by bleeding or sacrificing the animal. The serum may be used for research purposes in the form obtained from the animal or, in the alternative, the anti-Notum antibodies may be partially or fully purified to provide immunoglobulin fractions or homogeneous antibody preparations.

c. Monoclonal Antibodies

While polyclonal antibodies may be used in conjunction with certain aspects of the present invention, preferred embodiments comprise the use of Notum reactive monoclonal antibodies. As used herein, the term monoclonal antibody or mAb refers to an antibody obtained from a population of substantially homogeneous antibodies, i.e., the individual antibodies comprising the population are identical except for possible mutations, e.g., naturally occurring mutations, that may be present in minor amounts. Thus, the modifier monoclonal indicates the character of the antibody as not being a mixture of discrete antibodies and may be used in conjunction with any type of antibody. In certain embodiments, such a monoclonal antibody includes an antibody comprising a polypeptide sequence that binds or associates with Notum, wherein the Notum-binding polypeptide sequence was obtained by a process that includes the selection of a single target binding polypeptide sequence from a plurality of polypeptide sequences.

In preferred embodiments, antibody-producing cell lines are prepared from cells isolated from the immunized animal. After immunization, the animal is sacrificed and lymph node and/or splenic B cells are immortalized by means well known in the art. Methods of immortalizing cells include, but are not limited to, transfecting them with oncogenes, infecting them with an oncogenic virus and cultivating them under conditions that select for immortalized cells, subjecting them to carcinogenic or mutating compounds, fusing them with an immortalized cell, e.g., a myeloma cell, and inactivating a tumor suppressor gene. If fusion with myeloma cells is used, the myeloma cells preferably do not secrete immunoglobulin polypeptides (a non-secretory cell line). Immortalized cells are screened using Notum, or an immunoreactive portion thereof. In a preferred embodiment, the initial screening is performed using an enzyme-linked immunoassay (ELISA) or a radioimmunoassay.

More generally, discrete monoclonal antibodies consistent with the present invention can be prepared using a wide variety of techniques known in the art including hybridoma, recombinant techniques, phage display technologies, yeast libraries, transgenic animals (e.g. a XenoMouse® or HuMAb Mouse®) or some combination thereof. For example, monoclonal antibodies can be produced using hybridoma techniques such as broadly described above and taught in more detail in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) each of which is incorporated herein. Using the disclosed protocols, antibodies are preferably raised in mammals by multiple subcutaneous or intraperitoneal injections of the relevant antigen and an adjuvant. As previously discussed, this immunization generally elicits an immune response that comprises production of antigen-reactive antibodies (that may be fully human if the immunized animal is transgenic) from activated splenocytes or lymphocytes. While the resulting antibodies may be harvested from the serum of the animal to provide polyclonal preparations, it is generally more desirable to isolate individual lymphocytes from the spleen, lymph nodes or peripheral blood to provide homogenous preparations of monoclonal antibodies. Most typically, the lymphocytes are obtained from the spleen and immortalized to provide hybridomas.

For example, as described above, the selection process can be the selection of a unique clone from a plurality of clones, such as a pool of hybridoma clones, phage clones, or recombinant DNA clones. It should be understood that a selected Notum binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include discrete antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins that may be cross-reactive.

d. Chimeric Antibodies

In another embodiment, the antibody of the invention may comprise chimeric antibodies derived from covalently joined protein segments from at least two different species or types of antibodies. It will be appreciated that, as used herein, the term chimeric antibodies is directed to constructs in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). In one exemplary embodiment, a chimeric antibody in accordance with the teachings herein may comprise murine V_{H} and V_{L} amino acid sequences and constant regions derived from human sources. In other compatible embodiments a chimeric antibody of the present invention may comprise a CDR grafted or humanized antibody as described below.

Generally, a goal of making a chimeric antibody is to create a chimera in which the number of amino acids from the intended subject species is maximized. One example is the CDR-grafted antibody, in which the antibody comprises one or more complementarity determining regions (CDRs) from a particular species or belonging to a particular antibody class or subclass, while the remainder of the antibody chain(s) is/are identical with or homologous to a corresponding sequence in antibodies derived from another species or belonging to another antibody class or subclass. For use in humans, the variable region or selected CDRs from a rodent antibody often are grafted into a human antibody, replacing the naturally occurring variable regions or CDRs of the human antibody. These constructs generally have the advantages of providing full strength modulator functions (e.g., CDC, ADCC, etc.) while reducing unwanted immune responses to the antibody by the subject.

e. Humanized Antibodies

Similar to the CDR grafted antibody is a humanized antibody. Generally, a humanized antibody is produced from a monoclonal antibody raised initially in a non-human animal. As used herein humanized forms of non-human (e.g., murine) antibodies are chimeric antibodies that contain minimal sequence derived from non-human immunoglobulin. In one embodiment, a humanized antibody is a human immunoglobulin (recipient antibody) in which residues from a CDR of the recipient are replaced by residues from a CDR of a non-human species (donor antibody) such as mouse, rat, rabbit, or nonhuman primate having the desired specificity, affinity, and/or capacity.

In selected embodiments, the acceptor antibody may comprise consensus sequences. To create consensus human frameworks, frameworks from several human heavy chain or light chain amino acid sequences may be aligned to identify a consensus amino acid sequence. Moreover, in many instances, one or more framework residues in the variable domain of the human immunoglobulin are replaced by corresponding non-human residues from the donor antibody. These framework substitutions are identified by methods well known in the art, e.g., by modeling of the interactions of the CDR and framework residues to identify framework residues important for antigen binding and sequence comparison to identify unusual framework residues at particular positions. Such substitutions help maintain the appropriate three-dimensional configuration of the grafted CDR(s) and often improve infinity over similar constructs with no framework substitutions. Furthermore, humanized antibodies may comprise residues that are not found in the recipient antibody or in the donor antibody. These modifications may be made to further refine antibody performance using well-known techniques.

CDR grafting and humanized antibodies are described, for example, in U.S. Pat. Nos. 6,180,370, 5,693,762, 5,693,761, 5,585,089, and 5,530,101. In general, a humanized antibody will comprise substantially all of at least one, and typically two, variable domains, in which all or substantially all of the CDRs correspond to those of a non-human immunoglobulin, and all or substantially all of the framework regions are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1: 105-115 (1998); Harris, Biochem. Soc. Transactions 23:1035-1038 (1995); Rude and Gross, Curr. Op. Biotech. 5:428-433 (1994); and U.S. Pat. Nos. 6,982,321 and 7,087,409. Still another method is termed humaneering and is described, for example, in U.S. 2005/0008625. For the purposes of the present application the term humanized antibodies will be held to expressly include CDR grafted antibodies (i.e. human antibodies comprising one or more grafted non-human CDRs) with no or minimal framework substitutions.

Additionally, a non-human anti-Notum antibody may also be modified by specific deletion of human T cell epitopes or deimmunization by the methods disclosed in WO 98/52976 and WO 00/34317. Briefly, the heavy and light chain variable regions of an antibody can be analyzed for peptides that bind to MHC Class II; these peptides represent potential T-cell epitopes (as defined in WO 98/52976 and WO 00/34317). For detection of potential T-cell epitopes, a computer modeling approach termed peptide threading can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or by single amino acid substitutions. As far as possible, conservative substitutions are made. Often, but not exclusively, an amino acid common to a position in human germline antibody sequences may be used. After the deimmunizing changes are identified, nucleic acids encoding V_{H} and V_{L} can be constructed by mutagenesis or other synthetic methods (e.g., de novo synthesis, cassette replacement, and so forth). A mutagenized variable sequence can, optionally, be fused to a human constant region.

In selected embodiments, at least 60%, 65%, 70%, 75%, or 80% of the humanized antibody variable region residues will correspond to those of the parental framework region (FR) and CDR sequences. In other embodiments at least 85% or 90% of the humanized antibody residues will correspond to those of the parental framework region (FR) and CDR sequences. In a further preferred embodiment, greater than 95% of the humanized antibody residues will correspond to those of the parental framework region (FR) and CDR sequences.

Humanized antibodies may be fabricated using common molecular biology and biomolecular engineering techniques as described herein. These methods include isolating, manipulating, and expressing nucleic acid sequences that encode all or part of immunoglobulin Fv variable regions from at least one of a heavy or light chain. Sources of such nucleic acid are well known to those skilled in the art and, for example, may be obtained from a hybridoma, eukaryotic cell or phage producing an antibody or immunoreactive fragment against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al. (1992) J. Mol. Biol. 227:776-798; Cook, G. P. et al. (1995) Immunol. Today 16: 237-242; Chothia, D. et al. (1992) J. Mol. Bio. 227:799-817; and Tomlinson et al. (1995) EMBO J 14:4628-4638. The V BASE directory provides a comprehensive directory of human immunoglobulin variable region sequences (See Retter et al., (2005) Nuc Acid Res 33: 671-674). These sequences can be used as a source of human sequence, e.g., for framework regions and CDRs. As set forth herein consensus human framework regions can also be used, e.g., as described in U.S. Pat. No. 6,300,064.

f. Human Antibodies

In addition to the aforementioned antibodies, those skilled in the art will appreciate that the antibodies of the present invention may comprise fully human antibodies. For the purposes of the instant application the term human antibody comprises an antibody which possesses an amino acid sequence that corresponds to that of an antibody produced by a human and/or has been made using any of the techniques for making human antibodies as disclosed herein. This definition of a human antibody specifically excludes a humanized antibody comprising non-human antigen-binding residues.

Human antibodies can be produced using various techniques known in the art. As alluded to above, phage display techniques may be used to provide immunoactive binding regions in accordance with the present teachings. Thus, certain embodiments of the invention provide methods for producing anti-Notum antibodies or antigen-binding portions thereof comprising the steps of synthesizing a library of (preferably human) antibodies on phage, screening the library with Notum or an antibody-binding portion thereof, isolating phage that bind Notum, and obtaining the immunoreactive fragments from the phage. By way of example, one method for preparing the library of antibodies for use in phage display techniques comprises the steps of immunizing a non-human animal comprising human or non-human immunoglobulin loci with Notum or an antigenic portion thereof to create an immune response, extracting antibody-producing cells from the immunized animal; isolating RNA encoding heavy and light chains of antibodies of the invention from the extracted cells, reverse transcribing the RNA to produce cDNA, amplifying the cDNA using primers, and inserting the cDNA into a phage display vector such that antibodies are expressed on the phage. More particularly, DNA encoding the V_{H} and V_{L} domains are recombined together with an scFv linker by PCR and cloned into a phagemid vector (e.g., p CANTAB 6 or pComb 3 HSS). The vector may then be electroporated in E. coli and then the E. coli is infected with helper phage. Phage used in these methods are typically filamentous phage including fd and M13 and the V_{H} and V_{L} domains are usually recombinantly fused to either the phage gene III or gene VIII.

Recombinant human anti-Notum antibodies of the invention may be isolated by screening a recombinant combinatorial antibody library prepared as above. In a preferred embodiment, the library is a scFv phage display library, generated using human V_{L} and V_{H} cDNAs prepared from mRNA isolated from B cells. Methods for preparing and screening such libraries are well known in the art and kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology 9:1370-1372 (1991); Hay et al., Hum. Antibod. Hybridomas 3:81-85 (1992); Huse et al., Science 246:1275-1281 (1989); McCafferty et al., Nature 348:552-554 (1990); Griffiths et al., EMBO J. 12:725-734 (1993); Hawkins et al., J. Mol. Biol. 226:889-896 (1992); Clackson et al., Nature 352:624-628 (1991); Gram et al., Proc. Natl. Acad. Sci. USA 89:3576-3580 (1992); Garrad et al., Bio/Technology 9:1373-1377 (1991); Hoogenboom et al., Nuc. Acid Res. 19:4133-4137 (1991); and Barbas et al., Proc. Natl. Acad. Sci. USA 88:7978-7982 (1991).

The antibodies produced by naive libraries (either natural or synthetic) can be of moderate affinity (Kₐ of about 10⁶ to 10⁷ M⁻¹), but affinity maturation can also be mimicked in vitro by constructing and reselecting from secondary libraries as described in the art. For example, mutation can be introduced at random in vitro by using error-prone polymerase (reported in Leung et al., Technique, 1: 11-15 (1989)) in the method of Hawkins et al., J. Mol. Biol., 226: 889-896 (1992) or in the method of Gram et al., Proc. Natl. Acad. Sci. USA, 89: 3576-3580 (1992). Additionally, affinity maturation can be performed by randomly mutating one or more CDRs, e.g. using PCR with primers carrying random sequence spanning the CDR of interest, in selected individual Fv clones and screening for higher affinity clones. WO 9607754 described a method for inducing mutagenesis in a complementarity determining region of an immunoglobulin light chain to create a library of light chain genes. Another effective approach is to recombine the V_{H} or V_{L} domains selected by phage display with repertoires of naturally occurring V domain variants obtained from unimmunized donors and screen for higher affinity in several rounds of chain reshuffling as described in Marks et al., Biotechnol., 10: 779-783 (1992). This technique allows the production of antibodies and antibody fragments with a dissociation constant K_{d} (k_{off}/kₒₙ) of about 10⁻⁹M or less.

It will further be appreciated that similar procedures may be employed using libraries comprising eukaryotic cells (e.g., yeast) that express binding pairs on their surface. As with phage display technology, the eukaryotic libraries are screened against the antigen of interest (i.e., Notum) and cells expressing candidate-binding pairs are isolated and cloned. Steps may be taken to optimize library content and for affinity maturation of the reactive binding pairs. See, for example, U.S. Pat. No. 7,700,302 and U.S. Ser. No. 12/404,059. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. MoI. Biol, 227:381 (1991); Marks et al., J. MoI. Biol, 222:581 (1991)). In other embodiments human binding pairs may be isolated from combinatorial antibody libraries generated in eukaryotic cells such as yeast. See e.g., U.S. Pat. No. 7,700,302. Such techniques advantageously allow for the screening of large numbers of candidate modulators and provide for relatively easy manipulation of candidate sequences (e.g., by affinity maturation or recombinant shuffling).

Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and U.S. Pat. No. 6,075,181 and 6,150,584 regarding Xenomouse® technology along with the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B-lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., J. Immunol, 147 (1):86-95 (1991); and U.S. Pat. No. 5,750,373.

VI. Antibody Characteristics

No matter how obtained or which of the aforementioned forms the antibody modulator takes (e.g., humanized, human, etc.) the preferred embodiments of the disclosed modulators may exhibit various characteristics. In this regard anti-Notum antibody-producing cells (e.g., hybridomas or yeast colonies) may be selected, cloned and further screened for desirable characteristics including, for example, robust growth, high antibody production and, as discussed in more detail below, desirable antibody characteristics. Hybridomas can be expanded in vivo in syngeneic animals, in animals that lack an immune system, e.g., nude mice, or in cell culture in vitro. Methods of selecting, cloning and expanding hybridomas and/or colonies, each of which produces a discrete antibody species, are well known to those of ordinary skill in the art.

a. Neutralizing Antibodies

In particularly preferred embodiments the modulators of the instant invention will comprise neutralizing antibodies or derivative or fragment thereof. The term neutralizing antibody or neutralizing antagonist refers to an antibody or antagonist that binds to or interacts with a ligand or enzyme, prevents binding of the ligand or enzyme to its binding partner or substrate and interrupts the biological response that otherwise would result from the interaction of the two molecules. In assessing the binding and specificity of an antibody or immunologically functional fragment or derivative thereof, an antibody or fragment will substantially inhibit binding of a ligand or enzyme to its binding partner or substrate when an excess of antibody reduces the quantity of binding partner bound to the target molecule by at least about 20%, 30%, 40%, 50%, 60%, 70%, 80%, 85%, 90%, 95%, 97%, 99% or more (as measured in an in vitro competitive binding assay such as the TCF assay set forth in the Examples herein). In the case of antibodies to Notum, a neutralizing antibody or antagonist will diminish the ability of Notum to cleave GPI by at least about 20%, 30%, 40%, 50%, 60%, 70%, 80%, 85%, 90%, 95%, 97%, 99% or more and thereby reduce the concentration of free glypicans. It will be appreciated that this diminished concentration of glypicans may be measured directly using art recognized techniques or may be measured by the impact such reduction will have on Notum related pathways such as Wnt, Hh or BMP.

b. Internalizing Antibodies

While evidence indicates that Notum may be secreted by the cell, at least some Notum remains likely remains associated with the cell surface thereby allowing for internalization of the disclosed modulators. Accordingly, anti-Notum antibodies may be internalized, at least to some extent, by cells that express Notum. For example, an anti-Notum antibody that binds to Notum on the surface of a tumor-initiating cell may be internalized by the tumor-initiating cell. In particularly preferred embodiments such anti-Notum antibodies may be associated with or conjugated to cytotoxic moieties that kill the cell upon internalization.

As used herein, an anti-Notum antibody that internalizes is one that is taken up by the cell upon binding to Notum associated with a mammalian cell. The internalizing antibody includes antibody fragments, human or humanized antibody and antibody conjugates. Internalization may occur in vitro or in vivo. For therapeutic applications, internalization may occur in vivo. The number of antibody molecules internalized may be sufficient or adequate to kill a Notum-expressing cell, especially a Notum-expressing tumor initiating cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the tumor cell. Whether an anti-Notum antibody internalizes upon binding Notum on a mammalian cell can be determined by various assays including those described in the Examples below. Methods of detecting whether an antibody internalizes into a cell are described in U.S. Pat. No. 7,619,068 which is incorporated herein by reference in its entirety.

c. Depleting Antibodies

In other preferred embodiments the modulators of the instant invention will comprise depleting antibodies or derivative or fragment thereof. The term depleting antibody refers to an antibody or fragment that binds to or associates with Notum on or near the cell surface and induces, promotes or causes the death or elimination of the cell (e.g., by complement-dependent cytotoxicity or antibody-dependent cellular cytotoxicity). In some embodiments discussed more fully below the selected depleting antibodies will be associated or conjugated to a cytotoxic agent. Preferably a depleting antibody will be able to remove, eliminate or kill at least 20%, 30%, 40%, 50%, 60%, 70%, 80%, 85%, 90%, 95%, 97%, or 99% of tumor perpetuating cells in a defined cell population. In some embodiments the cell population may comprise enriched, sectioned, purified or isolated tumor perpetuating cells. In other embodiments the cell population may comprise whole tumor samples or heterogeneous tumor extracts that comprise tumor perpetuating cells. Those skilled in the art will appreciate that standard biochemical techniques as described in the Examples below may be used to monitor and quantify the depletion of tumor perpetuating cells in accordance with the teachings herein.

d. Epitope Binding

It will further be appreciated the disclosed anti-Notum antibodies will associate with, or bind to, discrete epitopes or determinants presented by the selected target(s). As used herein the term epitope refers to that portion of the target antigen capable of being recognized and specifically bound by a particular antibody. When the antigen is a polypeptide such as Notum, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. More specifically, the skilled artisan will appreciate the term epitope includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three dimensional structural characteristics, as well as specific charge characteristics. Additionally an epitope may be linear or conformational. In a linear epitope, all of the points of interaction between the protein and the interacting molecule (such as an antibody) occur linearly along the primary amino acid sequence of the protein. In a conformational epitope, the points of interaction occur across amino acid residues on the protein that are linearly separated from one another.

Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct competition studies to find antibodies that competitively bind with one another, i.e. the antibodies compete for binding to the antigen. A high throughput process for binning antibodies based upon their cross-competition is described in WO 03/48731.

As used herein, the term binning refers to a method to group antibodies based on their antigen binding characteristics. The assignment of bins is somewhat arbitrary, depending on how different the observed binding patterns of the antibodies tested. Thus, while the technique is a useful tool for categorizing antibodies of the instant invention, the bins do not always directly correlate with epitopes and such initial determinations should be further confirmed by other art recognized methodology.

With this caveat one can determine whether a selected primary antibody (or fragment thereof) binds to the same epitope or cross competes for binding with a second antibody by using methods known in the art and set forth in the Examples herein. In one embodiment, one allows the primary antibody of the invention to bind to Notum under saturating conditions and then measures the ability of the secondary antibody to bind to Notum. If the test antibody is able to bind to Notum at the same time as the primary anti-Notum antibody, then the secondary antibody binds to a different epitope than the primary antibody. However, if the secondary antibody is not able to bind to Notum at the same time, then the secondary antibody binds to the same epitope, an overlapping epitope, or an epitope that is in close proximity to the epitope bound by the primary antibody. As known in the art and detailed in the Examples below, the desired data can be obtained using solid phase direct or indirect radioimmunoassay (RIA), solid phase direct or indirect enzyme immunoassay (EIA), sandwich competition assay, a Biacore™ system (i.e., surface plasmon resonance—GE Healthcare), a ForteBio® Analyzer (i.e., bio-layer interferometry—ForteBio, Inc.) or flow cytometric methodology. The term surface plasmon resonance, as used herein, refers to an optical phenomenon that allows for the analysis of real-time biospecific interactions by detection of alterations in protein concentrations within a biosensor matrix. In a particularly preferred embodiment, the analysis is performed using a Biacore or ForteBio instrument as demonstrated in the Examples below.

The term compete when used in the context of antibodies that compete for the same epitope means competition between antibodies is determined by an assay in which the antibody or immunologically functional fragment under test prevents or inhibits specific binding of a reference antibody to a common antigen. Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Additional details regarding methods for determining competitive binding are provided in the Examples herein. Usually, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more.

Besides epitope specificity the disclosed antibodies may be characterized using a number of different physical characteristics including, for example, binding affinities, melting temperature (Tm), and isoelectric points.

e. Binding Affinity

In this respect, the present invention further encompasses the use of antibodies that have a high binding affinity for Notum. An antibody of the invention is said to specifically bind its target antigen when the dissociation constant K_{d} (k_{off}/kₒₙ) is ≦10⁻⁸M. The antibody specifically binds antigen with high affinity when the K_{d} is ≦5×10⁻⁹M, and with very high affinity when the K_{d} is ≦5×10⁻¹⁰M. In one embodiment of the invention, the antibody has a K_{d} of ≦10⁻⁹M and an off-rate of about 1×10⁻⁴/sec. In one embodiment of the invention, the off-rate is <1×10⁻⁵/sec. In other embodiments of the invention, the antibodies will bind to Notum with a K_{d} of between about 10⁻⁸M and 10⁻¹⁰M, and in yet another embodiment it will bind with a K_{d}≦2×10⁻¹⁰M. Still other selected embodiments of the present invention comprise antibodies that have a disassociation constant or K_{d} (k_{off}/kₒₙ) of less than 10⁻²M, less than 5×10⁻²M, less than 10⁻³M, less than 5×10⁻³M, less than 10⁴M, less than 5×10⁻⁴M, less than 10⁻⁵M, less than 5×10⁻⁵M, less than 10⁻⁶M, less than 5×10⁻⁶M, less than 10⁻⁷M, less than 5×10⁻⁷M, less than 10⁻⁸M, less than 5×10⁻⁸M, less than 10⁻⁹M, less than 5×10⁻⁹M, less than 10⁻¹⁰M, less than 5×10⁻¹⁰M, less than 10⁻¹¹M, less than 5×10⁻¹¹M, less than 10⁻¹²M, less than 5×10⁻¹²M, less than 10⁻¹³M, less than 5×10⁻¹³M, less than 10⁻¹⁴M, less than 5×10⁻¹⁴M, less than 10⁻¹⁵M or less than 5×10⁻¹⁵M.

In specific embodiments, an antibody of the invention that immunospecifically binds to Notum has an association rate constant or kₒₙ rate (Notum (Ab)+antigen (Ag) kₒₙ←Ab-Ag) of at least 10⁵M⁻¹ s⁻¹, at least 2×10⁵M⁻¹ s⁻¹, at least 5×10⁵M⁻¹ s⁻¹, at least 10⁶M⁻¹ s⁻¹, at least 5×10⁶M⁻¹ s⁻¹, at least 10⁷M⁻¹ s⁻¹, at least 5×10⁷M⁻¹ s⁻¹, or at least 10⁸M⁻¹ s⁻¹.

In another embodiment, an antibody of the invention that immunospecifically binds to Notum has a k_{off} rate (Notum (Ab)+antigen (Ag) k_{off}←Ab-Ag) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹ less than 10⁻⁷s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹s⁻¹ or less than 10⁻¹⁰ s⁻¹.

In other selected embodiments of the present invention anti-Notum antibodies will have an affinity constant or Kₐ (kₒₙ/k_{off}) of at least 10²M⁻¹, at least 5×10²M⁻¹, at least 10³M⁻¹, at least 5×10³M⁻¹, at least 10⁴M⁻¹, at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10¹⁰M⁻¹, at least 5×10¹⁰M⁻¹, at least 10¹¹M⁻¹, at least 5×10¹¹M⁻¹, at least 10¹²M⁻¹, at least 5×10¹²M⁻¹, at least 10¹³M⁻¹, at least 5×10¹³M⁻¹, at least 10¹⁴M⁻¹, at least 5×10¹⁴M⁻¹, at least 10¹⁵M⁻¹ or at least 5×10¹⁵M⁻¹.

f. Isoelectric Points

In addition to the aforementioned binding properties, anti-Notum antibodies and fragments thereof, like all polypeptides, have an Isoelectric Point (pI), which is generally defined as the pH at which a polypeptide carries no net charge. It is known in the art that protein solubility is typically lowest when the pH of the solution is equal to the isoelectric point (pI) of the protein. Therefore it is possible to optimize solubility by altering the number and location of ionizable residues in the antibody to adjust the pI. For example the pI of a polypeptide can be manipulated by making the appropriate amino acid substitutions (e.g., by substituting a charged amino acid such as a lysine, for an uncharged residue such as alanine). Without wishing to be bound by any particular theory, amino acid substitutions of an antibody that result in changes of the pI of said antibody may improve solubility and/or the stability of the antibody. One skilled in the art would understand which amino acid substitutions would be most appropriate for a particular antibody to achieve a desired pI.

The pI of a protein may be determined by a variety of methods including but not limited to, isoelectric focusing and various computer algorithms (see for example Bjellqvist et al., 1993, Electrophoresis 14:1023). In one embodiment, the pI of the anti-Notum antibodies of the invention is between is higher than about 6.5, about 7.0, about 7.5, about 8.0, about 8.5, or about 9.0. In another embodiment, the pI of the anti-Notum antibodies of the invention is between is higher than 6.5, 7.0, 7.5, 8.0, 8.5, or 9.0. In yet another embodiment, substitutions resulting in alterations in the pI of antibodies of the invention will not significantly diminish their binding affinity for Notum. As discussed in more detail below, it is specifically contemplated that the substitution(s) of the Fc region that result in altered binding to FcγR may also result in a change in the pI. In a preferred embodiment, substitution(s) of the Fc region are specifically chosen to effect both the desired alteration in FcγR binding and any desired change in pI. As used herein, the pI value is defined as the pI of the predominant charge form.

g. Thermal Stability

It will further be appreciated that the Tm of the Fab domain of an antibody can be a good indicator of the thermal stability of an antibody and may further provide an indication of the shelf-life. Tm is merely the temperature of 50% unfolding for a given domain or sequence. A lower Tm indicates more aggregation/less stability, whereas a higher Tm indicates less aggregation/more stability. Thus, antibodies or fragments or derivatives having higher Tm are preferable. Moreover, using art-recognized techniques it is possible to alter the composition of the anti-Notum antibodies or domains thereof to increase or optimize molecular stability. See, for example, U.S. Pat. No. 7,960,142. Thus, in one embodiment, the Fab domain of a selected antibody has a Tm value higher than at least 50° C., 55° C., 60° C., 65° C., 70° C., 75° C., 80° C., 85° C., 90° C., 95° C., 100° C., 105° C., 110° C., 115° C. or 120° C. In another embodiment, the Fab domain of an antibody has a Tm value higher than at least about 50° C., about 55° C., about 60° C., about 65° C., about 70° C., about 75° C., about 80° C., about 85° C., about 90° C., about 95° C., about 100° C., about 105° C., about 110° C., about 115° C. or about 120° C. Thermal melting temperatures (Tm) of a protein domain (e.g., a Fab domain) can be measured using any standard method known in the art, for example, by differential scanning calorimetry (see, e.g., Vermeer et al., 2000, Biophys. J. 78:394-404; Vermeer et al., 2000, Biophys. J. 79: 2150-2154 both incorporated herein by reference).

VII. Notum Modulator Fragments and Derivatives

Whether the agents of the present invention comprise intact fusion constructs, antibodies, fragments or derivatives, the selected modulators will react, bind, combine, complex, connect, attach, join, interact or otherwise associate with Notum and thereby provide the desired anti-neoplastic effects. Those of skill in the art will appreciate that modulators comprising anti-Notum antibodies interact or associate with Notum through one or more binding sites expressed on the antibody. More specifically, as used herein the term binding site comprises a region of a polypeptide that is responsible for selectively binding to a target molecule of interest (e.g., enzyme, antigen, ligand, receptor, substrate or inhibitor). Binding domains comprise at least one binding site (e.g. an intact IgG antibody will have two binding domains and two binding sites). Exemplary binding domains include an antibody variable domain, a receptor-binding domain of a ligand, a ligand-binding domain of a receptor or an enzymatic domain. For the purpose of the instant invention the enzymatically active region of Notum (e.g., as part of an Fc-notum fusion construct) may comprise a binding site for a substrate (e.g., a glypican).

a. Fragments

Regardless of which form of the modulator (e.g. chimeric, humanized, etc.) is selected to practice the invention, it will be appreciated that immunoreactive fragments of the same may be used in accordance with the teachings herein. In the broadest sense, the term antibody fragment comprises at least a portion of an intact antibody (e.g. a naturally occurring immunoglobulin). More particularly the term fragment refers to a part or portion of an antibody or antibody chain (or Notum molecule in the case of Fc fusions) comprising fewer amino acid residues than an intact or complete antibody or antibody chain. The term antigen-binding fragment refers to a polypeptide fragment of an immunoglobulin or antibody that binds antigen or competes with intact antibody (i.e., with the intact antibody from which they were derived) for antigen binding (i.e., specific binding). As used herein, the term fragment of an antibody molecule includes antigen-binding fragments of antibodies, for example, an antibody light chain (V_{L}), an antibody heavy chain (V_{H}), a single chain antibody (scFv), a F(ab′)2 fragment, a Fab fragment, an Fd fragment, an Fv fragment, single domain antibody fragments, diabodies, linear antibodies, single-chain antibody molecules and multispecific antibodies formed from antibody fragments. Similarly, an enzymatically active fragment of Notum comprises a portion of the Notum molecule that retains its ability to interact with Notum substrates and modify them (e.g., clip them) in a manner similar to that of an intact Notum (though maybe with somewhat less efficiency).

Those skilled in the art will appreciate fragments can be obtained via chemical or enzymatic treatment of an intact or complete modulator (e.g., antibody or antibody chain) or by recombinant means. In this regard, while various antibody fragments are defined in terms of the digestion of an intact antibody, one of skill will appreciate that such fragments may be synthesized de novo either chemically or by using recombinant DNA methodology. Thus, the term antibody, as used herein, explicitly includes antibodies or fragments or derivatives thereof either produced by the modification of whole antibodies or synthesized de novo using recombinant DNA methodologies.

More specifically, papain digestion of antibodies produces two identical antigen-binding fragments, called Fab fragments, each with a single antigen-binding site, and a residual Fc fragment, whose name reflects its ability to crystallize readily. Pepsin treatment yields an F(ab′)₂ fragment that has two antigen-binding sites and is still capable of cross-linking antigen. The Fab fragment also contains the constant domain of the light chain and the first constant domain (C_{H}1) of the heavy chain. Fab′ fragments differ from Fab fragments by the addition of a few residues at the carboxy terminus of the heavy-chain C_{H}1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known. See, e.g., Fundamental Immunology, W. E. Paul, ed., Raven Press, N.Y. (1999), for a more detailed description of other antibody fragments.

It will further be appreciated that an Fv fragment is an antibody fragment that contains a complete antigen recognition and binding site. This region is made up of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

In other embodiments an antibody fragment, for example, is one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

b. Derivatives

In another embodiment, it will further be appreciated that the modulators of the invention may be monovalent or multivalent (e.g., bivalent, trivalent, etc.). As used herein the term valency refers to the number of potential target (i.e., Notum) binding sites associated with an antibody. Each target binding site specifically binds one target molecule or specific position or locus on a target molecule. When an antibody of the instant invention comprises more than one target binding site (multivalent), each target binding site may specifically bind the same or different molecules (e.g., may bind to different ligands or different antigens, or different epitopes or positions on the same antigen). For the purposes of the instant invention, the subject antibodies will preferably have at least one binding site specific for human Notum. In one embodiment the antibodies of the instant invention will be monovalent in that each binding site of the molecule will specifically bind to a single Notum position or epitope. In other embodiments, the antibodies will be multivalent in that they comprise more than one binding site and the different binding sites specifically associate with more than a single position or epitope. In such cases the multiple epitopes may be present on the selected Notum polypeptide or a single epitope may be present on Notum while a second, different epitope may be present on another molecule or surface. See, for example, U.S.P.N. 2009/0130105.

As alluded to above, multivalent antibodies may immunospecifically bind to different epitopes of the desired target molecule or may immunospecifically bind to both the target molecule as well as a heterologous epitope, such as a heterologous polypeptide or solid support material. While preferred embodiments of the anti-Notum antibodies only bind two antigens (i.e. bispecific antibodies), antibodies with additional specificities such as trispecific antibodies are also encompassed by the instant invention. Examples of bispecific antibodies include, without limitation, those with one arm directed against Notum and the other arm directed against any other antigen (e.g., an modulator cell marker). Methods for making bispecific antibodies are known in the art. Traditional production of full-length bispecific antibodies is based on the coexpression of two immunoglobulin heavy chain-light chain pairs, where the two chains have different specificities (Millstein et al., 1983, Nature, 305:537-539). Other more sophisticated compatible multispecific constructs and methods of their fabrication are set forth in U.S.P.N. 2009/0155255.

In yet other embodiments, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and/or C_{H}3 regions. In one example, the first heavy-chain constant region (C_{H}1) containing the site necessary for light chain binding is present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when, the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm (e.g., Notum), and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., 1986, Methods in Enzymology, 121:210. According to another approach described in WO96/27011, a pair of antibody molecules can be engineered to maximize the percentage of heterodimers that are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory cavities of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies also include cross-linked or heteroconjugate antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

VIII. Notum Modulators—Constant Region Modifications

a. Fc Region and Fc Receptors

In addition to the various modifications, substitutions, additions or deletions to the variable or binding region of the disclosed modulators (e.g., Fc-Notum or anti-Notum antibodies) set forth above, those skilled in the art will appreciate that selected embodiments of the present invention may also comprise substitutions or modifications of the constant region (i.e. the Fc region). More particularly, it is contemplated that the Notum modulators of the invention may contain inter alia one or more additional amino acid residue substitutions, mutations and/or modifications which result in a compound with preferred characteristics including, but not limited to: altered pharmacokinetics, increased serum half life, increase binding affinity, reduced immunogenicity, increased production, altered Fc ligand binding, enhanced or reduced ADCC or CDC activity, altered glycosylation and/or disulfide bonds and modified binding specificity. In this regard it will be appreciated that these Fc variants may advantageously be used to enhance the effective anti-neoplastic properties of the disclosed modulators.

The term Fc region herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A functional Fc region possesses an effector function of a native sequence Fc region. Exemplary effector functions include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

Fc receptor or FcR describes a receptor that binds to the Fc region of an antibody. In some embodiments, an FcR is a native human FcR. In some embodiments, an FcR is one that binds an IgG antibody (a gamma receptor) and includes receptors of the FcγRI, Fc.RII, and FcγRIII subclasses, including allelic variants and alternatively spliced forms of those receptors. FcγII receptors include FcγRIIA (an activating receptor) and FcγRIIB (an inhibiting receptor), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FγRIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term FcR herein. The term Fc receptor or FcR also includes the neonatal receptor, FcRn, which, in certain instances, is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

b. Fc Functions

As used herein complement dependent cytotoxicity and CDC refer to the lysing of a target cell in the presence of complement. The complement activation pathway is initiated by the binding of the first component of the complement system (C1q) to a molecule, an antibody for example, complexed with a cognate antigen. To assess complement activation, a CDC assay, e.g. as described in Gazzano-Santoro et al., 1996, J. Immunol. Methods, 202:163, may be performed.

Further, antibody-dependent cell-mediated cytotoxicity or ADCC refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the target arm cytotoxic cells and are absolutely required for such killing. Lysis of the target cell is extracellular, requires direct cell-to-cell contact, and does not involve complement.

Notum modulator variants with altered FcR binding affinity or ADCC activity is one which has either enhanced or diminished FcR binding activity and/or ADCC activity compared to a parent or unmodified antibody or to a modulator comprising a native sequence Fc region. The modulator variant which displays increased binding to an FcR binds at least one FcR with better affinity than the parent or unmodified antibody or to a modulator comprising a native sequence Fc region. A variant which displays decreased binding to an FcR, binds at least one FcR with worse affinity than the parent or unmodified antibody or to a modulator comprising a native sequence Fc region. Such variants which display decreased binding to an FcR may possess little or no appreciable binding to an FcR, e.g., 0-20% binding to the FcR compared to a native sequence IgG Fc region, e.g. as determined techniques well known in the art.

As to FcRn, the antibodies of the instant invention also comprise or encompass Fc variants with modifications to the constant region that provide half-lives (e.g., serum half-lives) in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies (or Fc containing molecules) of the present invention in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies having increased in vivo half-lives can be generated by techniques known to those of skill in the art. For example, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311. Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001).

c. Glycosylation Modifications

In still other embodiments, glycosylation patterns or compositions of the antibodies of the invention are modified. More particularly, preferred embodiments of the present invention may comprise one or more engineered glycoforms, i.e., an altered glycosylation pattern or altered carbohydrate composition that is covalently attached to a molecule comprising an Fc region. Engineered glycoforms may be useful for a variety of purposes, including but not limited to enhancing or reducing effector function, increasing the affinity of the antibody for a target antigen or facilitating production of the antibody. In cases where reduced effector function is desired, it will be appreciated that the molecule may be engineered to express in an aglycosylated form. Such carbohydrate modifications can be accomplished by, for example, altering one or more sites of glycosylation within the antibody sequence. That is, one or more amino acid substitutions can be made that result in elimination of one or more variable region framework glycosylation sites to thereby eliminate glycosylation at that site (see e.g. U.S. Pat. Nos. 5,714,350 and 6,350,861. Conversely, enhanced effector functions or improved binding may be imparted to the Fc containing molecule by engineering in one or more additional glycosylation sites.

Additionally or alternatively, an Fc variant can be made that has an altered glycosylation composition, such as a hypofucosylated antibody having reduced amounts of fucosyl residues or an antibody having increased bisecting GlcNAc structures. These and similar altered glycosylation patterns have been demonstrated to increase the ADCC ability of antibodies. Engineered glycoforms may be generated by any method known to one skilled in the art, for example by using engineered or variant expression strains, by co-expression with one or more enzymes (for example N-acetylglucosaminyltransferase III (GnTI11)), by expressing a molecule comprising an Fc region in various organisms or cell lines from various organisms or by modifying carbohydrate(s) after the molecule comprising Fc region has been expressed. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342, Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. Nos. 10/277,370; 10/113,929; PCT WO 00/61739A1; PCT WO 01/292246A1; PCT WO 02/311140A1; PCT WO 02/30954A1; Potillegent™ technology (Biowa, Inc.); GlycoMAb™ glycosylation engineering technology (GLYCART biotechnology AG); WO 00061739; EA01229125; U.S.P.N. 2003/0115614; Okazaki et al., 2004, JMB, 336: 1239-49.

IX. Modulator Expression

a. Overview

DNA encoding the desired Notum modulators may be readily isolated and sequenced using conventional procedures (e.g., by using oligonucleotide probes that are capable of binding specifically to genes encoding antibody heavy and light chains). Isolated and subcloned hybridoma cells (or phage or yeast derived colonies) may serve as a preferred source of such DNA if the modulator is an antibody. If desired, the nucleic acid can further be manipulated as described herein to create agents including fusion proteins, or chimeric, humanized or fully human antibodies. More particularly, the isolated DNA (which may be modified) can be used to clone constant and variable region sequences for the manufacture antibodies as described in U.S. Pat. No. 7,709,611.

This exemplary method entails extraction of RNA from the selected cells, conversion to cDNA, and amplification by PCR using antibody specific primers. Suitable primers are well known in the art and, as exemplified herein, are readily available from numerous commercial sources. It will be appreciated that, to express a recombinant human or non-human antibody isolated by screening of a combinatorial library, the DNA encoding the antibody is cloned into a recombinant expression vector and introduced into host cells including mammalian cells, insect cells, plant cells, yeast, and bacteria. In yet other embodiments, the modulators are introduced into and expressed by simian COS cells, NSO cells, Chinese Hamster Ovary (CHO) cells or myeloma cells that do not otherwise produce the desired construct. As will be discussed in more detail below, transformed cells expressing the desired modulator may be grown up in relatively large quantities to provide clinical and commercial supplies of the fusion construct or immunoglobulin.

Whether the nucleic acid encoding the desired portion of the Notum modulator is obtained or derived from phage display technology, yeast libraries, hybridoma based technology, synthetically or from commercial sources, it is to be understood that the present invention explicitly encompasses nucleic acid molecules and sequences encoding Notum modulators including fusion proteins and anti-Notum antibodies or antigen-binding fragments or derivatives thereof. The invention further encompasses nucleic acids or nucleic acid molecules (e.g., polynucleotides) that hybridize under high stringency, or alternatively, under intermediate or lower stringency hybridization conditions (e.g., as defined below), to polynucleotides complementary to nucleic acids having a polynucleotide sequence that encodes a modulator of the invention or a fragment or variant thereof. The term nucleic acid molecule or isolated nucleic acid molecule, as used herein, is intended to include at least DNA molecules and RNA molecules. A nucleic acid molecule may be single-stranded or double-stranded, but preferably is double-stranded DNA. Moreover, the present invention comprises any vehicle or construct, incorporating such modulator encoding polynucleotide including, without limitation, vectors, plasmids, host cells, cosmids or viral constructs.

The term isolated nucleic acid means a that the nucleic acid was (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid that is available for manipulation by recombinant DNA techniques.

More specifically, nucleic acids that encode a modulator, including one or both chains of an antibody of the invention, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing are also provided. The nucleic acids can be any length. They can be, for example, 5, 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length, and/or can comprise one or more additional sequences, for example, regulatory sequences, and/or be part of a larger nucleic acid, for example, a vector. These nucleic acids can be single-stranded or double-stranded and can comprise RNA and/or DNA nucleotides, and artificial variants thereof (e.g., peptide nucleic acids). Nucleic acids encoding modulators of the invention, including antibodies or immunoreactive fragments or derivatives thereof, have preferably been isolated as described above.

b. Hybridization and Identity

As indicated, the invention further provides nucleic acids that hybridize to other nucleic acids under particular hybridization conditions. Methods for hybridizing nucleic acids are well known in the art. See, e.g., Current Protocols in Molecular Biology, John Wiley & Sons, N.Y. (1989), 6.3.1-6.3.6. For the purposes of the instant application, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequences that are at least 65, 70, 75, 80, 85, 90, 95, 98 or 99% identical to each other typically remain hybridized to each other. More generally, for the purposes of the instant disclosure the term substantially identical with regard to a nucleic acid sequence may be construed as a sequence of nucleotides exhibiting at least about 85%, or 90%, or 95%, or 97% sequence identity to the reference nucleic acid sequence.

The basic parameters affecting the choice of hybridization conditions and guidance for devising suitable conditions are set forth by, for example, Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the nucleic acid.

It will further be appreciated that nucleic acids may, according to the invention, be present alone or in combination with other nucleic acids, which may be homologous or heterologous. In preferred embodiments, a nucleic acid is functionally linked to expression control sequences that may be homologous or heterologous with respect to said nucleic acid. In this context the term homologous means that a nucleic acid is also functionally linked to the expression control sequence naturally and the term heterologous means that a nucleic acid is not functionally linked to the expression control sequence naturally.

c. Expression

A nucleic acid, such as a nucleic acid expressing RNA and/or protein or peptide, and an expression control sequence are functionally linked to one another, if they are covalently linked to one another in such a way that expression or transcription of said nucleic acid is under the control or under the influence of said expression control sequence. If the nucleic acid is to be translated into a functional protein, then, with an expression control sequence functionally linked to a coding sequence, induction of said expression control sequence results in transcription of said nucleic acid, without causing a frame shift in the coding sequence or said coding sequence not being capable of being translated into the desired protein or peptide.

The term expression control sequence comprises according to the invention promoters, ribosome binding sites, enhancers and other control elements that regulate transcription of a gene or translation of mRNA. In particular embodiments of the invention, the expression control sequences can be regulated. The exact structure of expression control sequences may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region that includes a promoter sequence for transcriptional control of the functionally linked nucleic acid. Expression control sequences may also comprise enhancer sequences or upstream activator sequences.

According to the invention the term promoter or promoter region relates to a nucleic acid sequence which is located upstream (5′) to the nucleic acid sequence being expressed and controls expression of the sequence by providing a recognition and binding site for RNA-polymerase. The promoter region may include further recognition and binding sites for further factors that are involved in the regulation of transcription of a gene. A promoter may control the transcription of a prokaryotic or eukaryotic gene. Furthermore, a promoter may be inducible and may initiate transcription in response to an inducing agent or may be constitutive if transcription is not controlled by an inducing agent. A gene that is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

Promoters which are preferred according to the invention include promoters for SP6, T3 and T7 polymerase, human U6 RNA promoter, CMV promoter, and artificial hybrid promoters thereof (e.g. CMV) where a part or parts are fused to a part or parts of promoters of genes of other cellular proteins such as e.g. human GAPDH (glyceraldehyde-3-phosphate dehydrogenase), and including or not including (an) additional intron(s).

According to the invention, the term expression is used in its most general meaning and comprises the production of RNA or of RNA and protein/peptide. It also comprises partial expression of nucleic acids. Furthermore, expression may be carried out transiently or stably.

In a preferred embodiment, a nucleic acid molecule is according to the invention present in a vector, where appropriate with a promoter, which controls expression of the nucleic acid. The term vector is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors may comprise plasmids, phagemids, bacteriophages or viral genomes. The term plasmid as used herein generally relates to a construct of extrachromosomal genetic material, usually a circular DNA duplex, which can replicate independently of chromosomal DNA.

In practicing the present invention it will be appreciated that many conventional techniques in molecular biology, microbiology, and recombinant DNA technology are optionally used. Such conventional techniques relate to vectors, host cells and recombinant methods as defined herein. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., supra Other useful references, e.g. for cell isolation and culture (e.g., for subsequent nucleic acid or protein isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla. Methods of making nucleic acids (e.g., by in vitro amplification, purification from cells, or chemical synthesis), methods for manipulating nucleic acids (e.g., site-directed mutagenesis, by restriction enzyme digestion, ligation, etc.), and various vectors, cell lines and the like useful in manipulating and making nucleic acids are described in the above references. In addition, essentially any polynucleotide (including, e.g., labeled or biotinylated polynucleotides) can be custom or standard ordered from any of a variety of commercial sources.

Thus, in one aspect, the present invention provides recombinant host cells allowing recombinant expression of antibodies of the invention or portions thereof. Antibodies produced by expression in such recombinant host cells are referred to herein as recombinant antibodies. The present invention also provides progeny cells of such host cells, and antibodies produced by the same.

The term recombinant host cell (or simply host cell), as used herein, means a cell into which a recombinant expression vector has been introduced. It should be understood that recombinant host cell and host cell mean not only the particular subject cell but also the progeny of such a cell. Because certain modifications may occur in succeeding generations due to either mutation or environmental influences, such progeny may not, in fact, be identical to the parent cell, but are still included within the scope of the term host cell as used herein. Such cells may comprise a vector according to the invention as described above.

In another aspect, the present invention provides a method for making an antibody or portion thereof as described herein. According to one embodiment, said method comprises culturing a cell transfected or transformed with a vector as described above, and retrieving the antibody or portion thereof.

As indicated above, expression of an antibody of the invention (or fragment or variants thereof) preferably comprises expression vector(s) containing a polynucleotide that encodes the desired anti-Notum antibody. Methods that are well known to those skilled in the art can be used to construct expression vectors comprising antibody coding sequences and appropriate transcriptional and translational control signals. These methods include, for example, in vitro recombinant DNA techniques, synthetic techniques, and in vivo genetic recombination. Embodiments of the invention, thus, provide replicable vectors comprising a nucleotide sequence encoding an anti-Notum antibody of the invention (e.g., a whole antibody, a heavy or light chain of an antibody, a heavy or light chain variable domain of an antibody, or a portion thereof, or a heavy or light chain CDR, a single chain Fv, or fragments or variants thereof), operably linked to a promoter. In preferred embodiments such vectors may include a nucleotide sequence encoding the heavy chain of an antibody molecule (or fragment thereof), a nucleotide sequence encoding the light chain of an antibody (or fragment thereof) or both the heavy and light chain.

Once the nucleotides of the present invention have been isolated and modified according to the teachings herein, they may be used to produce selected modulators including anti-Notum antibodies or fragments thereof.

X. Modulator Production and Purification

Using art recognized molecular biology techniques and current protein expression methodology, substantial quantities of the desired modulators may be produced. More specifically, nucleic acid molecules encoding modulators, such as antibodies obtained and engineered as described above, may be integrated into well known and commercially available protein production systems comprising various types of host cells to provide preclinical, clinical or commercial quantities of the desired pharmaceutical product. It will be appreciated that in preferred embodiments the nucleic acid molecules encoding the modulators are engineered into vectors or expression vectors that provide for efficient integration into the selected host cell and subsequent high expression levels of the desired Notum modulator.

Preferably nucleic acid molecules encoding Notum modulators and vectors comprising these nucleic acid molecules can be used for transfection of a suitable mammalian, plant, bacterial or yeast host cell though it will be appreciated that prokaryotic systems may be used for modulator production. Transfection can be by any known method for introducing polynucleotides into a host cell. Methods for the introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming mammalian cells are well known in the art. See, e.g., U.S. Pat. Nos. 4,399,216, 4,912,040, 4,740,461, and 4,959,455. Further, methods of transforming plant cells are well known in the art, including, e.g., Agrobacterium-mediated transformation, biolistic transformation, direct injection, electroporation and viral transformation. Methods of transforming bacterial and yeast cells are also well known in the art.

Moreover, the host cell may be co-transfected with two expression vectors of the invention, for example, the first vector encoding a heavy chain derived polypeptide and the second vector encoding a light chain derived polypeptide. The two vectors may contain identical selectable markers that enable substantially equal expression of heavy and light chain polypeptides. Alternatively, a single vector may be used which encodes, and is capable of expressing, both heavy and light chain polypeptides. In such situations, the light chain is preferably placed before the heavy chain to avoid an excess of toxic free heavy chain. The coding sequences for the heavy and light chains may comprise cDNA or genomic DNA.

a. Host-Expression Systems

A variety of host-expression vector systems, many commercially available, are compatible with the teachings herein and may be used to express the modulators of the invention. Such host-expression systems represent vehicles by which the coding sequences of interest may be expressed and subsequently purified, but also represent cells which may, when transformed or transfected with the appropriate nucleotide coding sequences, express a molecule of the invention in situ. Such systems include, but are not limited to, microorganisms such as bacteria (e.g., E. coli, B. subtilis, streptomyces) transformed with recombinant bacteriophage DNA, plasmid DNA or cosmid DNA expression vectors containing modulator coding sequences; yeast (e.g., Saccharomyces, Pichia) transfected with recombinant yeast expression vectors containing modulator coding sequences; insect cell systems infected with recombinant virus expression vectors (e.g., baculovirus) containing modulator coding sequences; plant cell systems (e.g., Nicotiana, Arabidopsis, duckweed, corn, wheat, potato, etc.) infected with recombinant virus expression vectors (e.g., cauliflower mosaic virus, CaMV; tobacco mosaic virus, TMV) or transfected with recombinant plasmid expression vectors (e.g., Ti plasmid) containing modulator coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the molecule being expressed. For example, when a large quantity of such a protein is to be produced, for the generation of pharmaceutical compositions of a modulator, vectors which direct the expression of high levels of fusion protein products that are readily purified may be desirable. Such vectors include, but are not limited to, the E. coli expression vector pUR278 (Ruther et al., EMBO 1. 2:1791 (1983)), in which the coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) may be used as a vector to express foreign genes. The virus grows in Spodoptera frugiperda cells. The coding sequences may be cloned individually into non-essential regions (for example, the polyhedrin gene) of the virus and placed under control of an AcNPV promoter (for example, the polyhedrin promoter).

In mammalian host cells, a number of viral-based expression systems may be used to introduce the desired nucleotide sequence. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (e.g., see Logan & Shenk, Proc. Natl. Acad. Sci. USA 8 1:355-359 (1984)). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)). Thus, compatible mammalian cell lines available as hosts for expression are well known in the art and include many immortalized cell lines available from the American Type Culture Collection (ATCC). These include, inter alia, Chinese hamster ovary (CHO) cells, NSO cells, SP2 cells, HEK-293T cells, 293 Freestyle cells (Life Technologies, San Diego), NIH-3T3 cells, HeLa cells, baby hamster kidney (BHK) cells, African green monkey kidney cells (COS), human hepatocellular carcinoma cells (e.g., Hep G2), A549 cells, and a number of other cell lines.

For long-term, high-yield production of recombinant proteins stable expression is preferred. Accordingly, cell lines that stably express the selected modulator may be engineered using standard art recognized techniques. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the molecule. Such engineered cell lines may be particularly useful in screening and evaluation of compositions that interact directly or indirectly with the molecule.

A number of selection systems are well known in the art and may be used including, but not limited to, the herpes simplex virus thymidine kinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:8 17 (1980)) genes can be employed in tk−, hgprt− or aprt− cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62: 191-217 (1993); TIB TECH 11(5):155-2 15 (May, 1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981). It will be appreciated that one particularly preferred method of establishing a stable, high yield cell line comprises the glutamine synthetase gene expression system (the GS system) which provides an efficient approach for enhancing expression under certain conditions. The GS system is discussed in whole or part in connection with EP patents 0 216 846, 0 256 055, 0 323 997 and 0 338 841 each of which is incorporated herein by reference.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function and/or purification of the protein. Different host cells have characteristic and specific mechanisms for the post-translational processing and modification of proteins and gene products. As known in the art appropriate cell lines or host systems can be chosen to ensure the desired modification and processing of the expressed polypeptide. To this end, eukaryotic host cells that possess the cellular machinery for proper processing of the primary transcript, glycosylation, and phosphorylation of the gene product are particularly effective for use in the instant invention. Accordingly, particularly preferred mammalian host cells include, but are not limited to, CHO, VERY, BHK, HeLa, COS, NSO, MDCK, 293, 3T3, W138, as well as breast cancer cell lines such as, for example, BT483, Hs578T, HTB2, BT2O and T47D, and normal mammary gland cell line such as, for example, CRL7O3O and HsS78Bst. Depending on the modulator and the selected production system, those of skill in the art may easily select and optimize appropriate host cells for efficient expression of the modulator.

b. Chemical Synthesis

Besides the aforementioned host cell systems, it will be appreciated that the modulators of the invention may be chemically synthesized using techniques known in the art (e.g., see Creighton, 1983, Proteins: Structures and Molecular Principles, W.H. Freeman & Co., N.Y., and Hunkapiller, M., et al., 1984, Nature 310:105-111). For example, a peptide corresponding to a polypeptide fragment of the invention can be synthesized by use of a peptide synthesizer. Furthermore, if desired, nonclassical amino acids or chemical amino acid analogs can be introduced as a substitution or addition into a polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

c. Transgenic Systems

The Notum modulators of the invention also can be produced transgenically through the generation of a mammal or plant that is transgenic for the immunoglobulin heavy and light chain sequences (or fragments or derivatives or variants thereof) of interest and production of the desired compounds in a recoverable form. In connection with the transgenic production in mammals, anti-Notum antibodies, for example, can be produced in, and recovered from, the milk of goats, cows, or other mammals. See, e.g., U.S. Pat. Nos. 5,827,690, 5,756,687, 5,750,172, and 5,741,957. In some embodiments, non-human transgenic animals that comprise human immunoglobulin loci are immunized with Notum or an immunogenic portion thereof, as described above. Methods for making antibodies in plants are described, e.g., in U.S. Pat. Nos. 6,046,037 and 5,959,177.

In accordance with the teachings herein non-human transgenic animals or plants may be produced by introducing one or more nucleic acid molecules encoding a Notum modulator of the invention into the animal or plant by standard transgenic techniques. See Hogan and U.S. Pat. No. 6,417,429. The transgenic cells used for making the transgenic animal can be embryonic stem cells or somatic cells or a fertilized egg. The transgenic non-human organisms can be chimeric, nonchimeric heterozygotes, and nonchimeric homozygotes. See, e.g., Hogan et al., Manipulating the Mouse Embryo: A Laboratory Manual 2nd ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999). In some embodiments, the transgenic non-human animals have a targeted disruption and replacement by a targeting construct that encodes, for example, a heavy chain and/or a light chain of interest. In one embodiment, the transgenic animals comprise and express nucleic acid molecules encoding heavy and light chains that specifically bind to Notum. While anti-Notum antibodies may be made in any transgenic animal, in particularly preferred embodiments the non-human animals are mice, rats, sheep, pigs, goats, cattle or horses. In further embodiments the non-human transgenic animal expresses the desired pharmaceutical product in blood, milk, urine, saliva, tears, mucus and other bodily fluids from which it is readily obtainable using art recognized purification techniques.

It is likely that modulators, including antibodies, expressed by different cell lines or in transgenic animals will have different glycosylation patterns from each other. However, all modulators encoded by the nucleic acid molecules provided herein, or comprising the amino acid sequences provided herein are part of the instant invention, regardless of the glycosylation state of the molecule, and more generally, regardless of the presence or absence of post-translational modification(s). In addition the invention encompasses modulators that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄, acetylation, formylation, oxidation, reduction, metabolic synthesis in the presence of tunicamycin, etc. Various post-translational modifications are also encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. Moreover, as set forth in the text and Examples below the polypeptides may also be modified with a detectable label, such as an enzymatic, fluorescent, radioisotopic or affinity label to allow for detection and isolation of the modulator.

d. Purification

Once a modulator of the invention has been produced by recombinant expression or any one of the other techniques disclosed herein, it may be purified by any method known in the art for purification of immunoglobulins, or more generally by any other standard technique for the purification of proteins. In this respect the modulator may be isolated. As used herein, an isolated Notum modulator is one that has been identified and separated and/or recovered from a component of its natural environment. Contaminant components of its natural environment are materials that would interfere with diagnostic or therapeutic uses for the polypeptide and may include enzymes, hormones, and other proteinaceous or nonproteinaceous solutes. Isolated modulators include a modulator in situ within recombinant cells because at least one component of the polypeptide's natural environment will not be present.

When using recombinant techniques, the Notum modulator (e.g. an anti-Notum antibody or derivative or fragment thereof) can be produced intracellularly, in the periplasmic space, or directly secreted into the medium. If the desired molecule is produced intracellularly, as a first step, the particulate debris, either host cells or lysed fragments, may be removed, for example, by centrifugation or ultrafiltration. For example, Carter, et al., Bio/Technology 10:163 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The modulator (e.g., fc-Notum or anti-Notum antibody) composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the selected construct. Protein A can be used to purify antibodies that are based on human IgG 1, IgG2 or IgG4 heavy chains (Lindmark, et al., J Immunol Meth 62:1 (1983)). Protein G is recommended for all mouse isotypes and for human IgG3 (Guss, et al., EMBO J 5:1567 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J.T. Baker; Phillipsburg, N.J.) is useful for purification. Other techniques for protein purification such as fractionation on an ion-exchange column, ethanol precipitation, reverse phase HPLC, chromatography on silica, chromatography on heparin, sepharose chromatography on an anion or cation exchange resin (such as a polyaspartic acid column), chromatofocusing, SDS-PAGE and ammonium sulfate precipitation are also available depending on the antibody to be recovered. In particularly preferred embodiments the modulators of the instant invention will be purified, at least in part, using Protein A or Protein G affinity chromatography.

XI. Conjugated Notum Modulators

Once the modulators of the invention have been purified according to the teachings herein they may be linked with, fused to, conjugated to (e.g., covalently or non-covalently) or otherwise associated with pharmaceutically active or diagnostic moieties or biocompatible modifiers. As used herein the term conjugate will be used broadly and held to mean any molecule associated with the disclosed modulators regardless of the method of association. In this respect it will be understood that such conjugates may comprise peptides, polypeptides, proteins, polymers, nucleic acid molecules, small molecules, mimetic agents, synthetic drugs, inorganic molecules, organic molecules and radioisotopes. Moreover, as indicated above the selected conjugate may be covalently or non-covalently linked to the modulator and exhibit various molar ratios depending, at least in part, on the method used to effect the conjugation.

In preferred embodiments it will be apparent that the modulators of the invention may be conjugated or associated with proteins, polypeptides or peptides that impart selected characteristics (e.g., biotoxins, biomarkers, purification tags, etc.). More generally, in selected embodiments the present invention encompasses the use of modulators or fragments thereof recombinantly fused or chemically conjugated (including both covalent and non-covalent conjugations) to a heterologous protein or polypeptide wherein the polypeptide comprises at least 10, at least 20, at least 30, at least 40, at least 50, at least 60, at least 70, at least 80, at least 90 or at least 100 amino acids. The construct does not necessarily need to be directly linked, but may occur through linker sequences. For example, antibodies may be used to target heterologous polypeptides to particular cell types expressing Notum, either in vitro or in vivo, by fusing or conjugating the modulators of the present invention to antibodies specific for particular cell surface receptors. Moreover, modulators fused or conjugated to heterologous polypeptides may also be used in in vitro immunoassays and may be compatible with purification methodology known in the art. See e.g., International publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452.

a. Biocompatible Modifiers

In a preferred embodiment, the modulators of the invention may be conjugated or otherwise associated with biocompatible modifiers that may be used to adjust, alter, improve or moderate modulator characteristics as desired. For example, antibodies or fusion constructs with increased in vivo half-lives can be generated by attaching relatively high molecular weight polymer molecules such as commercially available polyethylene glycol (PEG) or similar biocompatible polymers. Those skilled in the art will appreciate that PEG may be obtained in many different molecular weight and molecular configurations that can be selected to impart specific properties to the antibody (e.g. the half-life may be tailored). PEG can be attached to modulators or antibody fragments or derivatives with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity may be used. The degree of conjugation can be closely monitored by SDS-PAGE and mass spectrometry to ensure optimal conjugation of PEG molecules to antibody molecules. Unreacted PEG can be separated from antibody-PEG conjugates by, e.g., size exclusion or ion-exchange chromatography. In a similar manner, the disclosed modulators can be conjugated to albumin in order to make the antibody or antibody fragment more stable in vivo or have a longer half life in vivo. The techniques are well known in the art, see e.g., International Publication Nos. WO 93/15199, WO 93/15200, and WO 01/77137; and European Patent No. 0 413, 622. Other biocompatible conjugates are evident to those of ordinary skill and may readily be identified in accordance with the teachings herein.

b. Diagnostic or Detection Agents

In other preferred embodiments, modulators of the present invention, or fragments or derivatives thereof, are conjugated to a diagnostic or detectable agent which may be a biological molecule (e.g., a peptide or nucleotide) or a small molecule or radioisotope. Such modulators can be useful for monitoring the development or progression of a hyperproliferative disorder or as part of a clinical testing procedure to determine the efficacy of a particular therapy including the disclosed modulators. Such markers may also be useful in purifying the selected modulator, separating or isolating TIC or in preclinical procedures or toxicology studies.

Such diagnosis and detection can be accomplished by coupling the modulator to detectable substances including, but not limited to, various enzymes comprising for example horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I,), carbon (¹⁴C), sulfur (³⁵S), tritium (³H), indium (¹¹⁵In, ¹¹³In, ¹¹²In,), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, noradioactive paramagnetic metal ions, and molecules that are radiolabeled or conjugated to specific radioisotopes. In such embodiments appropriate detection methodology is well known in the art and readily available from numerous commercial sources.

As indicated above, in other embodiments the modulators or fragments thereof can be fused to marker sequences, such as a peptide or fluorophore to facilitate purification or diagnostic procedures such as immunohistochemistry or FACs. In preferred embodiments, the marker amino acid sequence is a hexa-histidine peptide, such as the tag provided in a pQE vector (Qiagen), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag (U.S. Pat. No. 4,703,004).

c. Therapeutic Moieties

As previously alluded to the modulators or fragments or derivatives thereof may also be conjugated, linked or fused to or otherwise associated with a therapeutic moiety such as a cytotoxin or cytotoxic agent, e.g., a cytostatic or cytocidal agent, a therapeutic agent or a radioactive metal ion, e.g., alpha or beta-emitters. As used herein a cytotoxin or cytotoxic agent includes any agent or therapeutic moiety that is detrimental to cells and may inhibit cell growth or survival. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4 (Immunogen, Inc.), dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Additional cytoxins comprise auristatins, including monomethyl auristatin E (MMAE) and monomethyl auristatin F (MMAF) (Seattle Genetics, Inc.), amanitins such as alpha-amanitin, beta-amanitin, gamma-amanitin or epsilon-amanitin (Heidelberg Pharma AG), DNA minor groove binding agents such as duocarmycin derivatives (Syntarga, B.V.) and modified pyrrolobenzodiazepine dimers (PBDs, Spirogen, Ltd). Furthermore, in one embodiment the Notum modulators of the instant invention may be associated with anti-CD3 binding molecules to recruit cytotoxic T-cells and have them target the tumor initiating cells (BiTE technology; see e.g., Fuhrmann, S. et. al. Annual Meeting of AACR Abstract No. 5625 (2010) which is incorporated herein by reference).

Additional compatible therapeutic moieties comprise cytotoxic agents including, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). A more extensive list of therapeutic moieties can be found in PCT publication WO 03/075957 and U.S.P.N. 2009/0155255 each of which is incorporated herein by reference.

The selected modulators can also be conjugated to therapeutic moieties such as radioactive materials or macrocyclic chelators useful for conjugating radiometal ions (see above for examples of radioactive materials). In certain embodiments, the macrocyclic chelator is 1,4,7,10-tetraazacyclododecane-N,M,N″,N″-tetraacetic acid (DOTA) which can be attached to the antibody via a linker molecule. Such linker molecules are commonly known in the art and described in Denardo et al., 1998, Clin Cancer Res. 4:2483; Peterson et al., 1999, Bioconjug. Chem. 10:553; and Zimmerman et al., 1999, Nucl. Med. Biol. 26:943.

Exemplary radioisotopes that may be compatible with this aspect of the invention include, but are not limited to, iodine (¹³¹I, ¹²⁵I, ¹²³I, ¹²¹I,), carbon (¹⁴C), copper (⁶²Cu, ⁶⁴Cu, ⁶⁷Cu), sulfur (³⁵S), tritium (³H), indium (¹¹⁵In, ¹¹³In, ¹¹²In, ¹¹¹In,), bismuth (²¹²Bi, ²¹³Bi), technetium (⁹⁹Tc), thallium (²⁰¹Ti) gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, ¹¹⁷Tin, ²²⁵Ac, ⁷⁶Br, and ²¹¹At. Other radionuclides are also available as diagnostic and therapeutic agents, especially those in the energy range of 60 to 4,000 keV. Depending on the condition to be treated and the desired therapeutic profile, those skilled in the art may readily select the appropriate radioisotope for use with the disclosed modulators.

Notum modulators of the present invention may also be conjugated to a therapeutic moiety or drug that modifies a given biological response. That is, therapeutic agents or moieties compatible with the instant invention are not to be construed as limited to classical chemical therapeutic agents. For example, in particularly preferred embodiments the drug moiety may be a protein or polypeptide or fragment thereof possessing a desired biological activity. Such proteins may include, for example, a toxin such as abrin, ricin A, Onconase (or another cytotoxic RNase), pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)). As set forth above, methods for fusing or conjugating modulators to polypeptide moieties are known in the art. In addition to the previously disclosed subject references see, e.g., U.S. Pat. Nos. 5,336,603; 5,622,929; 5,359,046; 5,349,053; 5,447,851, and 5,112,946; EP 307,434; EP 367,166; PCT Publications WO 96/04388 and WO 91/06570; Ashkenazi et al., 1991, PNAS USA 88:10535; Zheng et al., 1995, J Immunol 154:5590; and Vil et al., 1992, PNAS USA 89:11337 each of which is incorporated herein by reference. The association of a modulator with a moiety does not necessarily need to be direct, but may occur through linker sequences. Such linker molecules are commonly known in the art and described in Denardo et al., 1998, Clin Cancer Res 4:2483; Peterson et al., 1999, Bioconjug Chem 10:553; Zimmerman et al., 1999, Nucl Med Biol 26:943; Garnett, 2002, Adv Drug Deliv Rev 53:171 each of which is incorporated herein.

More generally, techniques for conjugating therapeutic moieties or cytotoxic agents to modulators are well known. Moieties can be conjugated to modulators by any art-recognized method, including, but not limited to aldehyde/Schiff linkage, sulphydryl linkage, acid-labile linkage, cis-aconityl linkage, hydrazone linkage, enzymatically degradable linkage (see generally Garnett, 2002, Adv Drug Deliv Rev 53:171). Also see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119. In preferred embodiments a Notum modulator that is conjugated to a therapeutic moiety or cytotoxic agent may be internalized by a cell upon binding to a Notum molecule associated with the cell surface thereby delivering the therapeutic payload.

XII. Diagnostics and Screening

As indicated, the present invention provides methods for detecting or diagnosing hyperproliferative disorders and methods of screening cells from a patient to identify a tumor initiating cell. Such methods include identifying an individual having cancer for treatment or monitoring progression of a cancer comprising contacting a sample obtained from a patient with a Notum modulator as described herein and detecting presence or absence, or level of association of the modulator to bound or free Notum in the sample. When the modulator comprises an antibody or immunologically active fragment thereof the association with Notum in the sample indicates that the sample may contain tumor perpetuating cells (e.g., a cancer stem cells) indicating that the individual having cancer may be effectively treated with a Notum modulator as described herein. The methods may further comprise a step of comparing the level of binding to a control. Conversely, when the selected modulator is Fc-Notum the enzymatic properties of the molecule as described herein may be monitored (directly or indirectly) when in contact with the sample to provide the desired information. Other diagnostic methods compatible with the teachings herein are well known in the art and can be practiced using commercial materials such as dedicated reporting systems.

Exemplary compatible assay methods include radioimmunoassays, enzyme immunoassays, competitive-binding assays, fluorescent immunoassay, immunoblot assays, Western Blot analysis, flow cytometry assays, and ELISA assays. More generally detection of Notum in a biological sample or the measurement of Notum enzymatic activity (or inhibition thereof) may be accomplished using any art-known assay.

In another aspect, and as discussed in more detail below, the present invention provides kits for detecting, monitoring or diagnosing a hyperproliferative disorder, identifying individual having such a disorder for possible treatment or monitoring progression (or regression) of the disorder in a patient, wherein the kit comprises a modulator as described herein, and reagents for detecting the impact of the modulator on a sample.

The Notum modulators and cells, cultures, populations and compositions comprising the same, including progeny thereof, can also be used to screen for or identify compounds or agents (e.g., drugs) that affect a function or activity of tumor initiating cells or progeny thereof by interacting with Notum (e.g., the polypeptide or genetic components thereof). The invention therefore further provides systems and methods for evaluation or identification of a compound or agent that can affect a function or activity tumor initiating cells or progeny thereof by associating with Notum or its substrates. Such compounds and agents can be drug candidates that are screened for the treatment of a hyperproliferative disorder, for example. In one embodiment, a system or method includes tumor initiating cells exhibiting Notum and a compound or agent (e.g., drug), wherein the cells and compound or agent (e.g., drug) are in contact with each other.

The invention further provides methods of screening and identifying Notum modulators or agents and compounds for altering an activity or function of tumor initiating cells or progeny cells. In one embodiment, a method includes contacting tumor initiating cells or progeny thereof with a test agent or compound; and determining if the test agent or compound modulates an activity or function of the Notum⁺ tumor initiating cells.

A test agent or compound modulating a Notum related activity or function of such tumor initiating cells or progeny thereof within the population identifies the test agent or compound as an active agent. Exemplary activity or function that can be modulated include changes in cell morphology, expression of a marker, differentiation or de-differentiation, maturation, proliferation, viability, apoptosis or cell death neuronal progenitor cells or progeny thereof.

Contacting, when used in reference to cells or a cell culture or method step or treatment, means a direct or indirect interaction between the composition (e.g., Notum⁺ cell or cell culture) and another referenced entity. A particular example of a direct interaction is physical interaction. A particular example of an indirect interaction is where a composition acts upon an intermediary molecule which in turn acts upon the referenced entity (e.g., cell or cell culture).

In this aspect of the invention modulates indicates influencing an activity or function of tumor initiating cells or progeny cells in a manner compatible with detecting the effects on cell activity or function that has been determined to be relevant to a particular aspect (e.g., metastasis or proliferation) of the tumor initiating cells or progeny cells of the invention. Exemplary activities and functions include, but are not limited to, measuring morphology, developmental markers, differentiation, proliferation, viability, cell respiration, mitochondrial activity, membrane integrity, or expression of markers associated with certain conditions. Accordingly, a compound or agent (e.g., a drug candidate) can be evaluated for its effect on tumor initiating cells or progeny cells, by contacting such cells or progeny cells with the compound or agent and measuring any modulation of an activity or function of tumor initiating cells or progeny cells as disclosed herein or would be known to the skilled artisan.

Methods of screening and identifying agents and compounds include those suitable for high throughput screening, which include arrays of cells (e.g., microarrays) positioned or placed, optionally at pre-determined locations or addresses. High-throughput robotic or manual handling methods can probe chemical interactions and determine levels of expression of many genes in a short period of time. Techniques have been developed that utilize molecular signals (e.g., fluorophores) and automated analyses that process information at a very rapid rate (see, e.g., Pinhasov et al., Comb. Chem. High Throughput Screen. 7:133 (2004)). For example, microarray technology has been extensively utilized to probe the interactions of thousands of genes at once, while providing information for specific genes (see, e.g., Mocellin and Rossi, Adv. Exp. Med. Biol. 593:19 (2007)).

Such screening methods (e.g., high-throughput) can identify active agents and compounds rapidly and efficiently. For example, cells can be positioned or placed (pre-seeded) on a culture dish, tube, flask, roller bottle or plate (e.g., a single multi-well plate or dish such as an 8, 16, 32, 64, 96, 384 and 1536 multi-well plate or dish), optionally at defined locations, for identification of potentially therapeutic molecules. Libraries that can be screened include, for example, small molecule libraries, phage display libraries, fully human antibody yeast display libraries (Adimab, LLC), siRNA libraries, and adenoviral transfection vectors.

XIII. Pharmaceutical Preparations and Therapeutic Uses

a. Formulations and Routes of Administration

Depending on the form of the modulator along with any optional conjugate, the mode of intended delivery, the disease being treated or monitored and numerous other variables, compositions of the instant invention may be formulated as desired using art recognized techniques. That is, in various embodiments of the instant invention compositions comprising Notum modulators are formulated with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents and the like, are also available. Certain non-limiting exemplary carriers include saline, buffered saline, dextrose, water, glycerol, ethanol, and combinations thereof.

More particularly it will be appreciated that, in some embodiments, the therapeutic compositions of the invention may be administered neat or with a minimum of additional components. Conversely the Notum modulators of the present invention may optionally be formulated to contain suitable pharmaceutically acceptable carriers comprising excipients and auxiliaries that are well known in the art and are relatively inert substances that facilitate administration of the modulator or which aid processing of the active compounds into preparations that are pharmaceutically optimized for delivery to the site of action. For example, an excipient can give form or consistency or act as a diluent to improve the pharmacokinetics of the modulator. Suitable excipients include but are not limited to stabilizing agents, wetting and emulsifying agents, salts for varying osmolarity, encapsulating agents, buffers, and skin penetration enhancers.

Disclosed modulators for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington, The Science and Practice of Pharmacy 20th Ed. Mack Publishing (2000). Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

Suitable formulations for enteral administration include hard or soft gelatin capsules, pills, tablets, including coated tablets, elixirs, suspensions, syrups or inhalations and controlled release forms thereof.

In general the compounds and compositions of the invention, comprising Notum modulators may be administered in vivo, to a subject in need thereof, by various routes, including, but not limited to, oral, intravenous, intra-arterial, subcutaneous, parenteral, intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

b. Dosages

Similarly, the particular dosage regimen, i.e., dose, timing and repetition, will depend on the particular individual and that individual's medical history. Empirical considerations, such as the half-life, generally will contribute to the determination of the dosage. Frequency of administration may be determined and adjusted over the course of therapy, and is based on reducing the number of hyperproliferative or neoplastic cells, including tumor initiating cells, maintaining the reduction of such neoplastic cells, reducing the proliferation of neoplastic cells, or delaying the development of metastasis. Alternatively, sustained continuous release formulations of a subject therapeutic composition may be appropriate. As alluded to above various formulations and devices for achieving sustained release are known in the art.

From a therapeutic standpoint the pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated. In general, the Notum modulators of the invention may be administered in an amount in the range of about 10 μg/kg body weight to about 100 mg/kg body weight per dose. In certain embodiments, the Notum modulators of the invention may be administered in an amount in the range of about 50 μg/kg body weight to about 5 mg/kg body weight per dose. In certain other embodiments, the Notum modulators of the invention may be administered in an amount in the range of about 100 μg/kg body weight to about 10 mg/kg body weight per dose. Optionally, the Notum modulators of the invention may be administered in an amount in the range of about 100 μg/kg body weight to about 20 mg/kg body weight per dose. Further optionally, the Notum modulators of the invention may be administered in an amount in the range of about 0.5 mg/kg body weight to about 20 mg/kg body weight per dose. In certain embodiments the compounds of present invention are provided a dose of at least about 100 μg/kg body weight, at least about 250 μg/kg body weight, at least about 750 μg/kg body weight, at least about 3 mg/kg body weight, at least about 5 mg/kg body weight, at least about 10 mg/kg body weight is administered.

Other dosing regimens may be predicated on Body Surface Area (BSA) calculations as disclosed in U.S. Pat. No. 7,744,877 which is incorporated herein by reference in its entirety. As is well known in the art the BSA is calculated using the patient's height and weight and provides a measure of a subject's size as represented by the surface area of his or her body. In selected embodiments of the invention using the BSA the modulators may be administered in dosages from 10 mg/m² to 800 mg/m². In other preferred embodiments the modulators will be administered in dosages from 50 mg/m² to 500 mg/m² and even more preferably at dosages of 100 mg/m², 150 mg/m², 200 mg/m², 250 mg/m², 300 mg/m², 350 mg/m², 400 mg/m² or 450 mg/m². Of course it will be appreciated that, regardless of how the dosages are calculated, multiple dosages may be administered over a selected time period to provide an absolute dosage that is substantially higher than the individual administrations.

In any event, the Notum modulators are preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like. Generally, an effective dose of the Notum modulator is administered to a subject one or more times. More particularly, an effective dose of the modulator is administered to the subject once a month, more than once a month, or less than once a month. In certain embodiments, the effective dose of the Notum modulator may be administered multiple times, including for periods of at least a month, at least six months, or at least a year.

Dosages and regimens may also be determined empirically for the disclosed therapeutic compositions in individuals who have been given one or more administration(s). For example, individuals may be given incremental dosages of a therapeutic composition produced as described herein. To assess efficacy of the selected composition, a marker of the specific disease, disorder or condition can be followed. In embodiments where the individual has cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or an antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

c. Combination Therapies

Combination therapies contemplated by the invention may be particularly useful in decreasing or inhibiting unwanted neoplastic cell proliferation (e.g. endothelial cells), decreasing the occurrence of cancer, decreasing or preventing the recurrence of cancer, or decreasing or preventing the spread or metastasis of cancer. In such cases the compounds of the instant invention may function as sensitizing or chemosensitizing agent by removing the TPC propping up and perpetuating the tumor mass (e.g. NTG cells) and allow for more effective use of current standard of care debulking or anti-cancer agents. That is, a combination therapy comprising an Notum modulator and one or more anti-cancer agents may be used to diminish established cancer e.g., decrease the number of cancer cells present and/or decrease tumor burden, or ameliorate at least one manifestation or side effect of cancer. As such, combination therapy refers to the administration of a Notum modulator and one or more anti-cancer agent that include, but are not limited to, cytotoxic agents, cytostatic agents, chemotherapeutic agents, targeted anti-cancer agents, biological response modifiers, immunotherapeutic agents, cancer vaccines, anti-angiogenic agents, cytokines, hormone therapies, radiation therapy and anti-metastatic agents.

According to the methods of the present invention, there is no requirement for the combined results to be additive of the effects observed when each treatment (e.g., anti-Notum antibody and anti-cancer agent) is conducted separately. Although at least additive effects are generally desirable, any increased anti-tumor effect above one of the single therapies is beneficial. Furthermore, the invention does not require the combined treatment to exhibit synergistic effects. However, those skilled in the art will appreciate that with certain selected combinations that comprise preferred embodiments, synergism may be observed.

To practice combination therapy according to the invention, a Notum modulator (e.g., anti-Notum antibody) in combination with one or more anti-cancer agent may be administered to a subject in need thereof in a manner effective to result in anti-cancer activity within the subject. The Notum modulator and anti-cancer agent are provided in amounts effective and for periods of time effective to result in their combined presence and their combined actions in the tumor environment as desired. To achieve this goal, the Notum modulator and anti-cancer agent may be administered to the subject simultaneously, either in a single composition, or as two or more distinct compositions using the same or different administration routes.

Alternatively, the modulator may precede, or follow, the anti-cancer agent treatment by, e.g., intervals ranging from minutes to weeks. In certain embodiments wherein the anti-cancer agent and the antibody are applied separately to the subject, the time period between the time of each delivery is such that the anti-cancer agent and modulator are able to exert a combined effect on the tumor. In a particular embodiment, it is contemplated that both the anti-cancer agent and the Notum modulator are administered within about 5 minutes to about two weeks of each other.

In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the modulator and the anti-cancer agent. The Notum modulator and one or more anti-cancer agent (combination therapy) may be administered once, twice or at least the period of time until the condition is treated, palliated or cured. Preferably, the combination therapy is administered multiple times. The combination therapy may be administered from three times daily to once every six months. The administering may be on a schedule such as three times daily, twice daily, once daily, once every two days, once every three days, once weekly, once every two weeks, once every month, once every two months, once every three months, once every six months or may be administered continuously via a minipump. As previously indicated the combination therapy may be administered via an oral, mucosal, buccal, intranasal, inhalable, intravenous, subcutaneous, intramuscular, parenteral, intratumor or topical route. The combination therapy may be administered at a site distant from the site of the tumor. The combination therapy generally will be administered for as long as the tumor is present provided that the combination therapy causes the tumor or cancer to stop growing or to decrease in weight or volume.

In one embodiment a Notum modulator is administered in combination with one or more anti-cancer agents for a short treatment cycle to a cancer patient to treat cancer. The duration of treatment with the antibody may vary according to the particular anti-cancer agent used. The invention also contemplates discontinuous administration or daily doses divided into several partial administrations. An appropriate treatment time for a particular anti-cancer agent will be appreciated by the skilled artisan, and the invention contemplates the continued assessment of optimal treatment schedules for each anti-cancer agent.

The present invention contemplates at least one cycle, preferably more than one cycle during which the combination therapy is administered. An appropriate period of time for one cycle will be appreciated by the skilled artisan, as will the total number of cycles, and the interval between cycles. The invention contemplates the continued assessment of optimal treatment schedules for each modulator and anti-cancer agent. Moreover, the invention also provides for more than one administration of either the anti-Notum antibody or the anti-cancer agent. The modulator and anti-cancer agent may be administered interchangeably, on alternate days or weeks; or a sequence of antibody treatment may be given, followed by one or more treatments of anti-cancer agent therapy. In any event, as will be understood by those of ordinary skill in the art, the appropriate doses of chemotherapeutic agents will be generally around those already employed in clinical therapies wherein the chemotherapeutics are administered alone or in combination with other chemotherapeutics.

In another preferred embodiment the Notum modulators of the instant invention may be used in maintenance therapy to reduce or eliminate the chance of tumor recurrence following the initial presentation of the disease. Preferably the disorder will have been treated and the initial tumor mass eliminated, reduced or otherwise ameliorated so the patient is asymptomatic or in remission. As such time the subject may be administered pharmaceutically effective amounts of the disclosed effectors one or more times even though there is little or no indication of disease using standard diagnostic procedures. In some embodiments the effectors will be administered on a regular schedule over a period of time. For example the Notum modulators could be administered weekly, every two weeks, monthly, every six weeks, every two months, every three months every six months or annually. Given the teachings herein one skilled in the art could readily determine favorable dosages and dosing regimens to reduce the potential of disease recurrence. Moreover such treatments could be continued for a period of weeks, months, years or even indefinitely depending on the patient response and clinical and diagnostic parameters.

In yet another preferred embodiment the effectors of the present invention may be used to prophylactically to prevent or reduce the possibility of tumor metastasis following a debulking procedure. As used in the instant disclosure a debulking procedure is defined broadly and shall mean any procedure, technique or method that eliminates, reduces, treats or ameliorates a tumor or tumor proliferation. Exemplary debulking procedures include, but are not limited to, surgery, radiation treatments (i.e., beam radiation), chemotherapy or ablation. At appropriate times readily determined by one skilled in the art in view of the instant disclosure the Notum modulators may be administered as suggested by clinical and diagnostic procedures to reduce tumor metastasis. The effectors may be administered one or more times at pharmaceutically effective dosages as determined using standard techniques. Preferably the dosing regimen will be accompanied by appropriate diagnostic or monitoring techniques that allow it to be modified as necessary.

d. Anti-Cancer Agents

As used herein the term anti-cancer agent means any agent that can be used to treat a cell proliferative disorder such as cancer, including cytotoxic agents, cytostatic agents, anti-angiogenic agents, debulking agents, chemotherapeutic agents, radiotherapy and radiotherapeutic agents, targeted anti-cancer agents, biological response modifiers, antibodies, and immunotherapeutic agents. It will be appreciated that, in selected embodiments as discussed above, anti-cancer agents may comprise conjugates and may be associated with modulators prior to administration.

The term cytotoxic agent means a substance that decreases or inhibits the function of cells and/or causes destruction of cells, i.e., the substance is toxic to the cells. Typically, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, gelonin, mitegellin, restrictocin, phenomycin, neomycin, and the tricothecenes) or animals, e.g., cytotoxic RNases, such as extracellular pancreatic RNases; DNase I, including fragments and/or variants thereof.

A chemotherapeutic agent means a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in the formulation CHOP.

Examples of anti-cancer agents that may be used in combination with (or conjugated to) the modulators of the present invention include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, an esperamicin, chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN″ doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluoromethylornithine (DMFO); retinoids; capecitabine; combretastatin; leucovorin (LV); oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators (SERMs), aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides; ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above. Other embodiments comprise the use of antibodies approved for cancer therapy including, but not limited to, rituximab, trastuzumab, gemtuzumab ozogamcin, alemtuzumab, ibritumomab tiuxetan, tositumomab, bevacizumab, cetuximab, patitumumab, ofatumumab, ipilimumab and brentuximab vedotin. Those skilled in the art will be able to readily identify additional anti-cancer agents that are compatible with the teachings herein.

e. Radiotherapy

The present invention also provides for the combination of Notum modulators with radiotherapy (i.e., any mechanism for inducing DNA damage locally within tumor cells such as gamma.-irradiation, X-rays, UV-irradiation, microwaves, electronic emissions and the like). Combination therapy using the directed delivery of radioisotopes to tumor cells is also contemplated, and may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

f. Neoplastic Conditions

Whether administered alone or in combination with an anti-cancer agent or radiotherapy, the Notum modulators of the instant invention are particularly useful for generally treating neoplastic conditions in patients or subjects which may include benign or malignant tumors (e.g., renal, liver, kidney, bladder, breast, gastric, ovarian, colorectal, prostate, pancreatic, lung, thyroid, hepatic carcinomas; sarcomas; glioblastomas; and various head and neck tumors); leukemias and lymphoid malignancies; other disorders such as neuronal, glial, astrocytal, hypothalamic and other glandular, macrophagal, epithelial, stromal and blastocoelic disorders; and inflammatory, angiogenic, immunologic disorders and disorders caused by pathogens. Particularly preferred targets for treatment with therapeutic compositions and methods of the present invention are neoplastic conditions comprising solid tumors. In other preferred embodiments the modulators of the present invention may be used for the diagnosis, prevention or treatment of hematologic malignancies. Preferably the subject or patient to be treated will be human although, as used herein, the terms are expressly held to comprise any mammalian species.

More specifically, neoplastic conditions subject to treatment in accordance with the instant invention may be selected from the group including, but not limited to, adrenal gland tumors, AIDS-associated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma). In certain preferred embodiments, the cancerous cells are selected from the group of solid tumors including but not limited to breast cancer, non-small cell lung cancer (NSCLC), small cell lung cancer, pancreatic cancer, colon cancer, prostate cancer, sarcomas, renal metastatic cancer, thyroid metastatic cancer, and clear cell carcinoma.

With regard to hematologic malignancies it will be further be appreciated that the compounds and methods of the present invention may be particularly effective in treating a variety of B-cell lymphomas, including low grade/NHL follicular cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, Waldenstrom's Macroglobulinemia, lymphoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic, follicular, diffuse large cell, diffuse small cleaved cell, large cell immunoblastic lymphoblastoma, small, non-cleaved, Burkitt's and non-Burkitt's, follicular, predominantly large cell; follicular, predominantly small cleaved cell; and follicular, mixed small cleaved and large cell lymphomas, See, Gaidono et al., “Lymphomas”, IN CANCER: PRINCIPLES & PRACTICE OF ONCOLOGY, Vol. 2: 2131-2145 (DeVita et al., eds., 5.sup.th ed. 1997). It should be clear to those of skill in the art that these lymphomas will often have different names due to changing systems of classification, and that patients having lymphomas classified under different names may also benefit from the combined therapeutic regimens of the present invention.

In yet other preferred embodiments the Notum modulators may be used to effectively treat certain myeloid and hematologic malignancies including leukemias such as chronic lymphocytic leukemia (CLL or B-CLL). CLL is predominantly a disease of the elderly that starts to increase in incidence after fifty years of age and reaches a peak by late sixties. It generally involves the proliferation of neoplastic peripheral blood lymphocytes. Clinical finding of CLL involves lymphocytosis, lymphadenopatliy, splenomegaly, anemia and thrombocytopenia. A characteristic feature of CLL is monoclonal B cell proliferation and accumulation of B-lymphocytes arrested at an intermediate state of differentiation where such B cells express surface IgM (sIgM) or both sIgM and sIgD, and a single light chain at densities lower than that on the normal B cells. However, as discussed above and shown in the Examples appended hereto, selected Notum expression (e.g., Notum) is upregulated on B-CLL cells thereby providing an attractive target for the disclosed modulators.

The present invention also provides for a preventative or prophylactic treatment of subjects who present with benign or precancerous tumors. It is not believed that any particular type of tumor or neoplastic disorder should be excluded from treatment using the present invention. However, the type of tumor cells may be relevant to the use of the invention in combination with secondary therapeutic agents, particularly chemotherapeutic agents and targeted anti-cancer agents.

As discussed herein, preferred embodiments of the instant invention comprise the use of Notum modulators to treat subjects suffering from solid tumors. In such subjects many of these solid tumors comprise tissue exhibiting various genetic mutations that may render them particularly susceptible to treatment with the disclosed effectors. For example, KRAS, APC and CTNNB1 mutations are relatively common in patients with colorectal cancer. Moreover, patients suffering from tumors with these mutations are usually the most refractory to current therapies; especially those patients with KRAS mutations. KRAS activating mutations, which typically result in single amino acid substitutions, are also implicated in other difficult to treat malignancies, including lung adenocarcinoma, mucinous adenoma, and ductal carcinoma of the pancreas.

Currently, the most reliable prediction of whether colorectal cancer patients will respond to EGFR- or VEGF-inhibiting drugs, for example, is to test for certain KRAS “activating” mutations. KRAS is mutated in 35-45% of colorectal cancers, and patients whose tumors express mutated KRAS do not respond well to these drugs. For example, KRAS mutations are predictive of a lack of response to panitumumab and cetuximab therapy in colorectal cancer (Lievre et al. Cancer Res 66:3992-5; Karapetis et al. NEJM 359:1757-1765). Approximately 85% of patients with colorectal cancer have mutations in the APC gene (Markowitz & Bertagnolli. NEJM 361:2449-60), and more than 800 APC mutations have been characterized in patients with familial adenomatous polyposis and colorectal cancer. A majority of these mutations result in a truncated APC protein with reduced functional ability to mediate the destruction of beta-catenin. Mutations in the beta-catenin gene, CTNNB1, can also result in increased stabilization of the protein, resulting in nuclear import and subsequent activation of several oncogenic transcriptional programs, which is also the mechanism of oncogenesis resulting from failure of mutated APC to appropriately mediate beta-catenin destruction, which is required to keep normal cell proliferation and differentiation programs in check. As indicated by the Examples herein, tumors comprising such mutations may prove to be particularly susceptible to treatment with the Notum modulators of the instant invention.

XIV. Articles of Manufacture

Pharmaceutical packs and kits comprising one or more containers, comprising one or more doses of a Notum modulator are also provided. In certain embodiments, a unit dosage is provided wherein the unit dosage contains a predetermined amount of a composition comprising, for example, an anti-Notum antibody, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water. In certain preferred embodiments, the composition comprises one or more substances that inhibit protein aggregation, including, but not limited to, sucrose and arginine. Any label on, or associated with, the container(s) indicates that the enclosed composition is used for diagnosing or treating the disease condition of choice.

The present invention also provides kits for producing single-dose or multi-dose administration units of a Notum modulator and, optionally, one or more anti-cancer agents. The kit comprises a container and a label or package insert on or associated with the container. Suitable containers include, for example, bottles, vials, syringes, etc. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). Such kits will generally contain in a suitable container a pharmaceutically acceptable formulation of the Notum modulator and, optionally, one or more anti-cancer agents in the same or different containers. The kits may also contain other pharmaceutically acceptable formulations, either for diagnosis or combined therapy. For example, in addition to the Notum modulator of the invention such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents. Such kits may also provide appropriate reagents to conjugate the Notum modulator with an anti-cancer agent or diagnostic agent (e.g., see U.S. Pat. No. 7,422,739 which is incorporated herein by reference in its entirety).

More specifically the kits may have a single container that contains the Notum modulator, with or without additional components, or they may have distinct containers for each desired agent. Where combined therapeutics are provided for conjugation, a single solution may be pre-mixed, either in a molar equivalent combination, or with one component in excess of the other. Alternatively, the Notum modulator and any optional anti-cancer agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody and any optional components to an animal or patient, e.g., one or more needles or syringes, or even an eye dropper, pipette, or other such like apparatus, from which the formulation may be injected or introduced into the animal or applied to a diseased area of the body. The kits of the present invention will also typically include a means for containing the vials, or such like, and other component in close confinement for commercial sale, such as, e.g., injection or blow-molded plastic containers into which the desired vials and other apparatus are placed and retained. Any label or package insert indicates that the Notum modulator composition is used for treating cancer, for example colorectal cancer.

XV. Research Reagents

Other preferred embodiments of the invention also exploit the properties of the disclosed modulators as an instrument useful for identifying, isolating, sectioning or enriching populations or subpopulations of tumor initiating cells through methods such as fluorescent activated cell sorting (FACS), magnetic activated cell sorting (MACS) or laser mediated sectioning. Those skilled in the art will appreciate that the modulators may be used in several compatible techniques for the characterization and manipulation of TIC including cancer stem cells (e.g., see U.S. Ser. Nos. 12/686,359, 12/669,136 and 12/757,649 each of which is incorporated herein by reference in its entirety).

XVI. Miscellaneous

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In addition, ranges provided in the specification and appended claims include both end points and all points between the end points. Therefore, a range of 2.0 to 3.0 includes 2.0, 3.0, and all points between 2.0 and 3.0.

Generally, nomenclature used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well known and commonly used in the art.

All references or documents disclosed or cited within this specification are, without limitation, incorporated herein by reference in their entirety. Moreover, any section headings used herein are for organizational purposes only and are not to be construed as limiting the subject matter described.

### EXAMPLES

The present invention, thus generally described, will be understood more readily by reference to the following Examples, which are provided by way of illustration and are not intended to be limiting of the instant invention. The Examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

### Example 1

### Characterization of Tumor Initiating Cell Populations

To characterize the cellular heterogeneity of solid tumors as they exist in cancer patients, elucidate the identity of tumor perpetuating cells (TPC; i.e. cancer stem cells: CSC) using particular phenotypic markers and identify clinically relevant therapeutic targets, a large non-traditional xenograft (NTX) tumor bank was developed and maintained using art recognized techniques. The NTX tumor bank, comprising a large number of discrete tumor cell lines, was propagated in immunocompromised mice through multiple passages of heterogeneous tumor cells originally obtained from numerous cancer patients afflicted by a variety of solid tumor malignancies. The continued availability of a large number of discrete early passage NTX tumor cell lines having well defined lineages greatly facilitate the identification and isolation of TPC as they allow for the reproducible and repeated characterization of cells purified from the cell lines. More particularly, isolated or purified TPC are most accurately defined retrospectively according to their ability to generate phenotypically and morphologically heterogeneous tumors in mice that recapitulate the patient tumor sample from which the cells originated. Thus, the ability to use small populations of isolated cells to generate fully heterogeneous tumors in mice is strongly indicative of the fact that the isolated cells comprise TPC. In such work the use of minimally passaged NTX cell lines greatly simplifies in vivo experimentation and provides readily verifiable results. Moreover, early passage NTX tumors also respond to therapeutic agents such as irinotecan (i.e. Camptosar®), which provides clinically relevant insights into underlying mechanisms driving tumor growth, resistance to current therapies and tumor recurrence.

As the NTX tumor cell lines were established the constituent tumor cell phenotypes were analyzed using flow cytometry to identify discrete markers that might be used to characterize, isolate, purify or enrich tumor initiating cells (TIC) and separate or analyze TPC and TProg cells within such populations. In this regard the inventors employed a proprietary proteomic based platform (i.e. PhenoPrint™ Array) that provided for the rapid characterization of cells based on protein expression and the concomitant identification of potentially useful markers. The PhenoPrint Array is a proprietary proteomic platform comprising hundreds of discrete binding molecules, many obtained from commercial sources, arrayed in 96 well plates wherein each well contains a distinct antibody in the phycoerythrin fluorescent channel and multiple additional antibodies in different fluorochromes arrayed in every well across the plate. This allows for the determination of expression levels of the antigen of interest in a subpopulation of selected tumor cells through rapid inclusion of relevant cells or elimination of non-relevant cells via non-phycoerythrin channels. When the PhenoPrint Array was used in combination with tissue dissociation, transplantation and stem cell techniques well known in the art (Al-Hajj et al., 2004, Dalerba et al., 2007 and Dylla et al., 2008, all supra, each of which is incorporated herein by reference in its entirety), it was possible to effectively identify relevant markers and subsequently isolate and transplant specific human tumor cell subpopulations with great efficiency.

Accordingly, upon establishing various NTX tumor cell lines as is commonly done for human tumors in severely immune compromised mice, the tumors were resected from mice upon reaching 800-2,000 mm³ and the cells were dissociated into single cell suspensions using art-recognized enzymatic digestion techniques (See for example U.S.P.N. 2007/0292414 which is incorporated herein). Data obtained from these suspensions using the PhenoPrint Array provided both absolute (per cell) and relative (vs. other cells in the population) surface protein expression on a cell-by-cell basis, leading to more complex characterization and stratification of cell populations. More specifically, use of the PhenoPrint Array allowed for the rapid identification of proteins or markers that prospectively distinguished TIC or TPC from NTG bulk tumor cells and tumor stroma and, when isolated from NTX tumor models, provided for the relatively rapid characterization of tumor cell subpopulations expressing differing levels of specific cell surface proteins. In particular, proteins with heterogeneous expression across the tumor cell population allow for the isolation and transplantation of distinct, and highly purified, tumor cell subpopulations expressing either high and low levels of a particular protein or marker into immune-compromised mice, thereby facilitating the assessment of whether TPC were enriched in one subpopulation or another.

The term enriching is used synonymously with isolating cells and means that the yield (fraction) of cells of one type is increased over the fraction of other types of cells as compared to the starting or initial cell population. Preferably, enriching refers to increasing the percentage by about 10%, by about 20%, by about 30%, by about 40%, by about 50% or greater than 50% of one type of cell in a population of cells as compared to the starting population of cells.

As used herein a marker, in the context of a cell or tissue, means any characteristic in the form of a chemical or biological entity that is identifiably associated with, or specifically found in or on a particular cell, cell population or tissue including those identified in or on a tissue or cell population affected by a disease or disorder. As manifested, markers may be morphological, functional or biochemical in nature. In preferred embodiments the marker is a cell surface antigen that is differentially or preferentially expressed by specific cell types (e.g., TPC) or by cells under certain conditions (e.g., during specific points of the cell life cycle or cells in a particular niche). Preferably, such markers are proteins, and more preferably, possess an epitope for antibodies, aptamers or other binding molecules as known in the art. However, a marker may consist of any molecule found on the surface or within a cell including, but not limited to, proteins (peptides and polypeptides), lipids, polysaccharides, nucleic acids and steroids. Examples of morphological marker characteristics or traits include, but are not limited to, shape, size, and nuclear to cytoplasmic ratio. Examples of functional marker characteristics or traits include, but are not limited to, the ability to adhere to particular substrates, ability to incorporate or exclude particular dyes, for example but not limited to exclusions of lipophilic dyes, ability to migrate under particular conditions and the ability to differentiate along particular lineages. Markers can also be a protein expressed from a reporter gene, for example a reporter gene expressed by the cell as a result of introduction of the nucleic acid sequence encoding the reporter gene into the cell and its transcription resulting in the production of the reporter protein that can be used as a marker. Such reporter genes that can be used as markers are, for example but not limited to fluorescent proteins enzymes, chromomeric proteins, resistance genes and the like.

In a related sense the term marker phenotype in the context of a tissue, cell or cell population (e.g., a stable TPC phenotype) means any marker or combination of markers that may be used to characterize, identify, separate, isolate or enrich a particular cell or cell population. In specific embodiments, the marker phenotype is a cell surface phenotype that may be determined by detecting or identifying the expression of a combination of cell surface markers.

Those skilled in the art will recognize that numerous markers (or their absence) have been associated with various populations of cancer stem cells and used to isolate or characterize tumor cell subpopulations. In this respect exemplary cancer stem cell markers comprise OCT4, Nanog, STAT3, EPCAM, CD24, CD34, NB84, TrkA, GD2, CD133, CD20, CD56, CD29, B7H3, CD46, transferrin receptor, JAM3, carboxypeptidase M, ADAM9, oncostatin M, Lgr5, Lgr6, CD324, CD325, nestin, Sox1, Bmi-1, eed, easyh1, easyh2, mf2, yy1, smarcA3, smarckA5, smarcD3, smarcE1, mllt3, FZD1, FZD2, FZD3, FZD4, FZD6, FZD7, FZD8, FZD9, FZD10, WNT2, WNT2B, WNT3, WNT5A, WNT10B, WNT16, AXIN1, BCL9, MYC, (TCF4) SLC7A8, IL1RAP, TEM8, TMPRSS4, MUC16, GPRC5B, SLC6A14, SLC4A11, PPAP2C, CAV1, CAV2, PTPN3, EPHA1, EPHA2, SLC1A1, CX3CL1, ADORA2A, MPZL1, FLJ10052, C4.4A, EDG3, RARRES1, TMEPAI, PTS, CEACAM6, NID2, STEAP, ABCA3, CRIM1, IL1R1, OPN3, DAF, MUC1, MCP, CPD, NMA, ADAM9, GJA1, SLC19A2, ABCA1, PCDH7, ADCY9, SLC39A1, NPC1, ENPP1, N33, GPNMB, LY6E, CELSR1, LRP3, C20orf52, TMEPAI, FLVCR, PCDHA10, GPR54, TGFBR3, SEMA4B, PCDHB2, ABCG2, CD166, AFP, BMP-4,13-catenin, CD2, CD3, CD9, CD14, CD31, CD38, CD44, CD45, CD74, CD90, CXCR4, decorin, EGFR, CD105, CD64, CD16, CD16a, CD16b, GLI1, GLI2, CD49b, and CD49f. See, for example, Schulenburg et al., 2010, PMID: 20185329, U.S. Pat. No. 7,632,678 and U.S.P.Ns. 2007/0292414, 2008/0175870, 2010/0275280, 2010/0162416 and 2011/0020221 each of which is incorporated herein by reference. It will be appreciated that a number of these markers were included in the PhenoPrint Array described above.

Similarly, non-limiting examples of cell surface phenotypes associated with cancer stem cells of certain tumor types include CD44⁺CD24^{low}, ALM⁺, CD133⁺, CD123⁺, CD34⁺CD38⁻, CD44⁺CD24⁻, CD46⁺CD324⁺CD66c⁻, CD133⁺CD34⁺CD10⁻CD19⁻, CD138⁻CD34⁻CD19⁺, CD133⁺RC2⁺, CD44⁺α₂β₁^{hi}CD133⁺, CD44⁺CD24⁺ESA⁺, CD271⁺, ABCB5⁺ as well as other cancer stem cell surface phenotypes that are known in the art. See, for example, Schulenburg et al., 2010, supra, Visvader et al., 2008, PMID: 18784658 and U.S.P.N. 2008/0138313, each of which is incorporated herein in its entirety by reference. Those skilled in the art will appreciate that marker phenotypes such as those exemplified immediately above may be used in conjunction with standard flow cytometric analysis and cell sorting techniques to characterize, isolate, purify or enrich TIC and/or TPC cells or cell populations for further analysis. Of interest with regard to the instant invention CD46, CD324 and, optionally, CD66c are either highly or heterogeneously expressed on the surface of many human colorectal (“CR”), breast (“BR”), non-small cell lung (NSCLC), small cell lung (SCLC), pancreatic (“PA”), melanoma (“MeI”), ovarian (“OV”), and head and neck cancer (“HN”) tumor cells, regardless of whether the tumor specimens being analyzed were primary patient tumor specimens or patient-derived NTX tumors.

### Example 2

### Isolation and Analysis of RNA Samples from Enriched Tumor Initiating Cell Populations

An established colorectal NTX cell line (SCRx-CR4) was used to initiate tumors in immune compromised mice. Once the mean tumor burden reached ˜300 mm³, mice were randomized and treated with either 15 mg/kg irinotecan or vehicle control (PBS) twice weekly for a period of twenty days, at which point in time the mice were euthanized and TPC, TProg, and NTG cells, respectively, were isolated from freshly resected NTX tumors generally using marker phenotypes as set forth in Example 1. More particularly, cell populations were isolated by fluorescence activated cell sorting (FACS) using CD46, CD324 and CD66c markers and immediately pelleted and lysed in Qiagen RLTPIus RNA lysis buffer (Qiagen, Inc.). The lysates were then stored at −80° C. until used. Upon thawing the RNA cell lysate, total RNA was extracted using the Qiagen RNEasy isolation kit (Qiagen, Inc.) following the vendor's instructions and quantified on the Nanodrop (Thermo Scientific) and a Bioanalyzer 2100 (Agilent) again using the vendor's protocols and recommended instrument settings. The resulting total RNA preparation was suitable for genetic sequencing and analysis.

The RNA samples obtained from the TPC, TProg and NTG cell populations isolated as described above from vehicle or irinotecan-treated mice were prepared for whole transcriptome sequencing using an Applied Biosystems SOLiD 3.0 (Sequencing by Oligo Ligation/Detection) next generation sequencing platform (Life Technologies), starting with 5 ng of total RNA per sample. The data generated by the SOLiD platform mapped to 34,609 genes from the human genome, was able to detect Notum and provided verifiable measurements of Notum levels in all samples.

Generally the SOLiD3 next generation sequencing platform enables parallel sequencing of clonally-amplified RNA/DNA fragments linked to beads. Sequencing by ligation with dye-labeled oligonucleotides is then used to generate 50 base reads of each fragment that exists in the sample with a total of greater than 50 million reads generating a much more accurate representation of the mRNA transcript level expression of proteins in the genome. The SOLiD3 platform is able to capture not only expression, but SNPs, known and unknown alternative splicing events, and potentially new exon discoveries based solely on the read coverage (reads mapped uniquely to genomic locations). Thus, use of this next generation platform allowed the determination of differences in transcript level expression as well as differences or preferences for specific splice variants of those expressed mRNA transcripts. Moreover, analysis with the SOLiD3 platform using a modified whole transcriptome protocol from Applied Biosystems only required approximately 5 ng of starting material pre-amplification. This is significant as extraction of total RNA from sorted cell populations where the TPC subset of cells is, for example, vastly smaller in number than the NTG or bulk tumors and thus results in very small quantities of usable starting material.

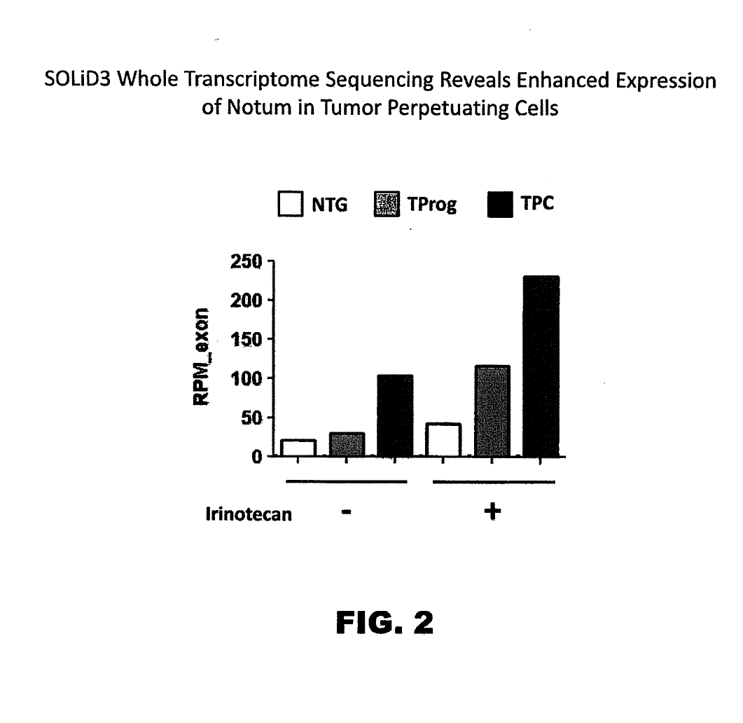

Duplicate runs of sequencing data from the SOLiD3 platform were normalized and transformed and fold ratios calculated as is standard industry practice. As seen in FIG. 2, an analysis of the data showed that Notum was up-regulated at the transcript level by 2 to 5 fold in the TPC over the TProg and NTG populations and was further elevated in NTX tumor-bearing mice being treated with 15 mg/kg irinotecan, twice weekly. The observed overexpression of Notum in the TPC subpopulation of NTX tumor samples using the extremely sensitive SOLiD3 analytical platform suggests that Notum may play an important role in colorectal tumorigenesis and maintenance.

### Example 3

### Real-Time PCR Analysis of Notum in Enriched Tumor Initiating Cell Populations

To confirm enhanced expression of Notum in TPC populations versus TProg and NTG cells, TaqMan quantitative real-time PCR was used to measure gene expression levels in respective cell populations isolated from various NTX lines as set forth above. It will be appreciated that such real-time PCR analysis allows for a more direct and rapid measurement of gene expression levels for discrete targets using primers and probe sets specific to a particular gene of interest. TaqMan real-time quantitative PCR was performed on an Applied Biosystems 5900HT Machine (Life Technologies) which was used to measure Notum gene expression in multiple patient-derived NTX line cell populations and corresponding controls. Subsequent analysis was conducted as specified in the instructions supplied with the TaqMan System and using commercially available Notum primer/probe sets (Life Technologies).

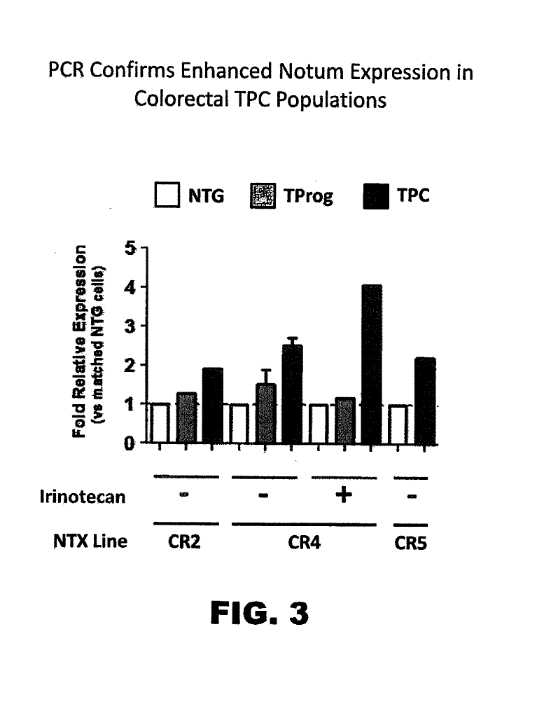

As seen in FIG. 3 quantitative real-time PCR interrogating gene expression in NTG, TProg and TPC populations isolated from 3 distinct colorectal NTX tumor lines (e.g., CR2, CR4 and CR5) shows that Notum gene expression is elevated approximately 2-fold in TPC cells, and this expression is further elevated to approximately 4-fold in mice undergoing treatment with irinotecan. The observation of elevated Notum expression in NTX TPC cell preparations as compared with TProg and NTG cell controls using the more widely accepted methodology of real-time quantitative PCR confirms the SOLiD3 whole transcriptome sequencing data of the previous Example and further implicates Notum as a driving factor in colorectal neoplasias. Moreover, increased Notum expression in tumors treated with an anti-cancer agent shows that Notum modulators or antagonists may prove valuable as an adjunct therapy.

### Example 4

### Expression of Notum in Unfractionated Colorectal Tumor Samples

In light of the fact that Notum gene expression was found to be elevated in TPC populations from colorectal tumors when compared with TProg and NTG cells, experiments were conducted to determine whether Notum expression levels were also elevated in unfractionated colorectal tumor samples versus normal adjacent tissue (NAT) and other normal tissue samples. Custom Tumor Scan qPCR (Origene Technologies) 384-well arrays containing 110 colorectal patient tumor specimens, normal adjacent tissue, and 48 normal tissues were designed and custom fabricated according to a provided protocol. Using the procedures detailed in Example 3 and the same Notum specific primer/probe sets, TaqMan real-time quantitative PCR was then performed in the wells of the custom plates.

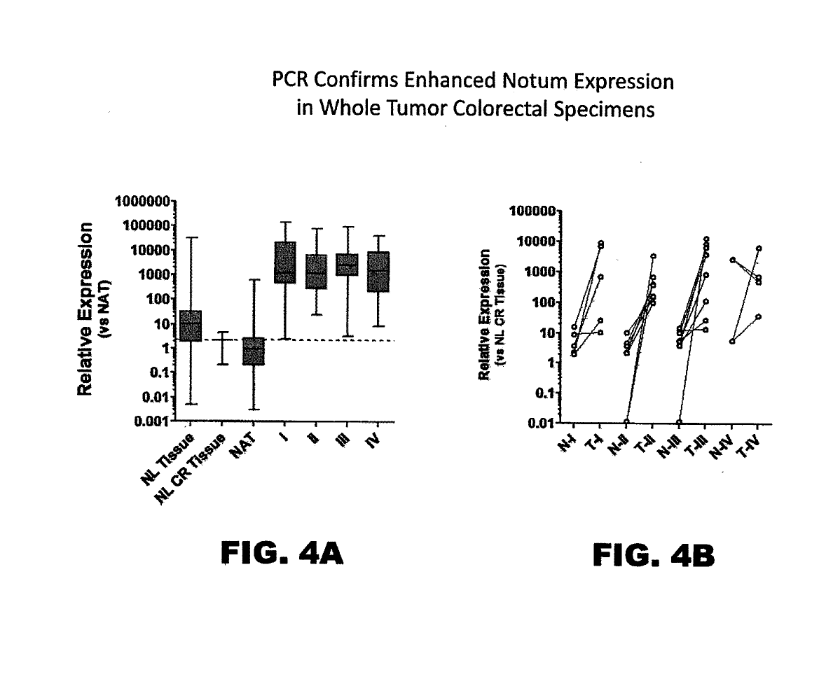

FIGS. 4A and 4B show the results of the expression data in a graphical format normalized against the mean expression in normal colon and rectum tissue. More specifically, FIG. 4A summarizes data generated using 168 tissue specimens, obtained from 110 colorectal cancer patients, (35 tissue specimens of which are normal adjacent tissue from colorectal cancer patients) and 48 normal tissues. In the plot data is represented as box and whisker plots, with the median value represented as a line within the box. Similarly, FIG. 4B contains data from 24 matched colorectal patient specimens obtained from tumor or normal adjacent tissue. Here the plotted data is presented on a sample by sample basis with linkage between the respective tumor and NAT. Both FIGS. 4A and 4B indicate that, in all four stages presented, the expressed level of the Notum gene is elevated in colorectal tumors and in matched tumor specimens versus normal adjacent tissue.

More particularly the results of real-time PCR on these primary patient tumor samples (as opposed to NTX tumors) showed that Notum gene expression was approximately 1,000-fold higher in the patient tumors versus normal adjacent tissue (NAT), irrespective of cancer stage (i.e. Stage I-IV disease). Notum gene expression was similarly elevated approximately 10-100 fold in matched tumor versus NAT. Moreover, Notum expression was relatively low in most normal tissues, with only normal placenta and liver tissue containing gene expression levels at or above the median levels observed in colorectal cancer patient tumors clustered by stage. Elevated expression of Notum in unfractionated colorectal tumor samples and relatively low expression levels in normal control tissue is again suggestive as to the role of the Notum gene product in the development and support of malignancies.

### Example 5

### Differential Expression of Notum in Exemplary Tumor Samples

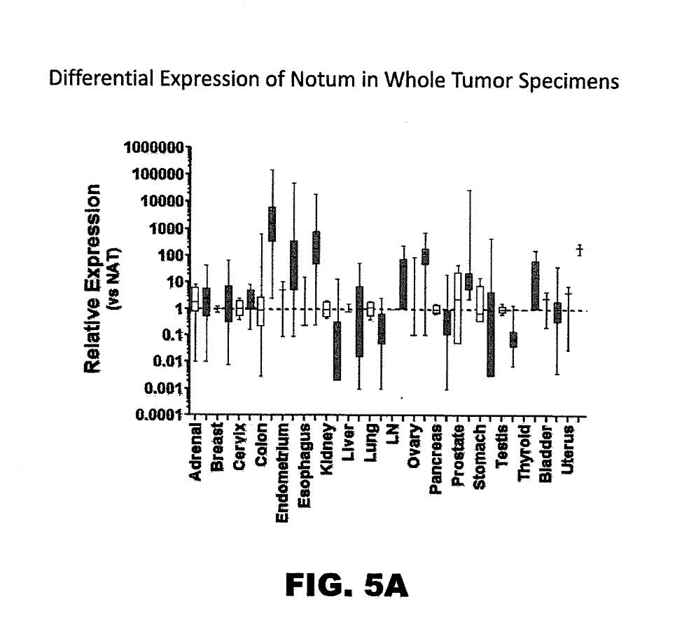

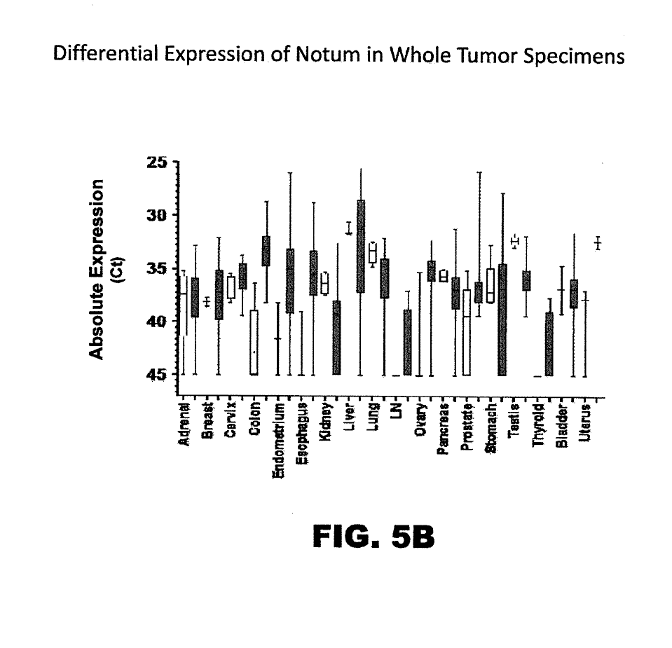

To further assess Notum gene expression in additional colorectal cancer patient tumor samples and tumor specimens from patients diagnosed with 1 of 17 other different solid tumor types, TaqMan qRT-PCR was performed using TissueScan qPCR (Origene Technologies) 384-well arrays, which were custom assembled according to a provided protocol as in Example 4. The results of the measurements are presented in FIGS. 5A and 5B and show that gene expression of Notum is significantly elevated in a number of tumor samples.

In this regard, FIGS. 5A and 5B show the relative or absolute gene expression levels, respectively, of human Notum in whole tumor specimens (grey box) or matched NAT (white box) from patients with one of eighteen different solid tumor types. In FIG. 5A, data is normalized against mean NAT gene expression for each tumor type analyzed. In FIG. 5B, the absolute expression of Notum was assessed in various tissues/tumors, with the data being plotted as the number of cycles (Ct) needed to reach exponential amplification by quantitative real-time PCR. Specimens not amplified were assigned a Ct value of 45, which represents the last cycle of amplification in the experimental protocol. Data is represented as box and whisker plots, with the median value represented as a line within the box.

In addition to patients diagnosed with colorectal cancer, those diagnosed with endometrial, esophageal and uterine cancer also had significantly more Notum gene expression in their tumors versus NAT, suggesting that Notum might also play a pathological role by impacting TPC self-renewal and proliferation in these tumors. Ovarian, prostate and thyroid tumors also had elevated Notum expression, albeit less significant. What was also clear from the these studies is that Notum gene expression was generally low to non-detectable in most NAT samples; with the highest expression being observed in the liver, testis and lung. Again, these data suggest that Notum expression is indicative, and potentially dispositive, as to tumorigenesis or perpetuation in a number of hyperproliferative disorders.

### Example 6

### Differential Notum Protein Expression in Various Pooled Tissue Lysates

After documenting enhanced Notum gene expression in a number of tumorigenic samples as evidenced by the previous Examples, evidence was sought for corresponding increases in the Notum protein in similar tumor samples. In this respect, reverse phase protein arrays comprising two pooled replicates of lysates from eleven different tumor types or their respective normal adjacent tissue were provided along with controls of 293 cells with or without TP53-overexpression as driven by an exogenous promoter (OriGene Technologies). Notum protein expression in the lysates was detected using a mouse polyclonal antibody generated against human Notum and colorimetric detection reagents and protocols provided by the manufacturer. Spots on the fabricated array were converted to a digital image using a flatbed scanner and then quantified using the SpotDenso function within AlphaEaseFc Software (Alpha Innotech, Inc).

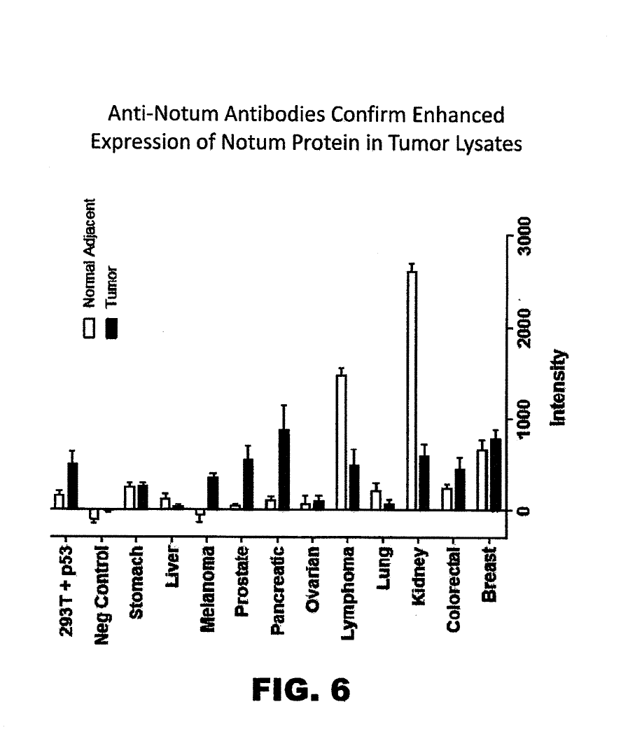

The results of these assays are shown in FIG. 6 and indicate that expression of the Notum protein is upregulated in several different types of tumor. More specifically, FIG. 6 shows the levels of expression of human Notum in normal adjacent tissue and 293T P53 negative controls (white) or 293T P53 positive controls and tumor tissue (black) from specimens obtained from patients with one of eleven different tumor types (i.e., primary tumor samples). Data was generated as described above and represented as average pixel intensity per spot. Data plotted represents Mean±SEM.

In addition to colorectal cancer, Notum protein expression appears significantly elevated in tumor specimens from patients with melanoma, prostate and pancreatic cancer. These data suggest that Notum overexpression may be involved in TPC proliferation and/or survival in these tumors. Furthermore, detection of Notum protein may be prognostic of these diseases.

In view of the forgoing Examples showing Notum is overexpressed in TPC enriched cell populations and various tumors (both at a genetic and proteomic level) coupled with the likelihood that such elevated expression levels are associated with tumorigenesis and tumor propagation, it was decided to construct Notum immunogens that could be used in the generation of Notum modulators.

### Example 7

### Construction and Expression of Tagged Notum Modulators

Constructs were fabricated and expressed as set forth below for use in generating Notum modulators. As a starting point a human Notum cDNA encoding the entire open reading frame (ORF) SEQ ID NO: 1 was obtained from a commercial source (Open Biosystems; Accession No. BC060882). The cDNA clone ORF sequence was confirmed by DNA sequencing to be without mutation relative to the reference sequence (GenBank NM_{—}178493).

For ease of purification and detection of the recombinant product, the cDNA encoding the full length Notum ORF was modified by PCR to include sequences encoding 8×-Hisand Strep-tag II epitopes, (IBA GmBH). The DNA encoding the modified Notum ORF was purified from the PCR using QiaQuick PCR clean up columns (Qiagen), the DNA subcloned between the Not I and Xho I sites of pCMV-Script (Stratagene, Inc.), and confirmed to be free of mutations by DNA sequencing. In this case, the wild-type Notum signal peptide sequence directs secretion of the recombinant protein.

In accordance with the present invention pSEC expression vectors were constructed for use in production of desired recombinant products. The pSEC-CAG expression vector contains the CAG promoter, which is composed of a human cytomegalovirus (CMV) major immediate-early gene enhancer/promoter region a β-globin/IgG chimeric intron located downstream of the enhancer/promoter region. pSEC-CAG vectors promotes strong, constitutive expression of cloned cDNA inserts in many cell types. pSEC-CAG also contains the IgK signal peptide/leader sequence to promote enhanced secretion of expressed of recombinant proteins from cells transfected with the plasmid. The epitope-tagged Notum ORF from pCMV-Script was subcloned by PCR into the pSEC-CAG vector between the Sfi I and Xho I sites to create pSEC-CAG-NOTUM-StrepHis.

pSEC-CAG-NOTUM-StrepHis DNA was used for 1 liter transfection of suspension 293 cells, and the recombinant protein was purified from supernatant of transfected cells using Nickel-NTA columns. More specifically, recombinant Notum protein was produced in adherent HEK293T cells, by transfecting the plasmid pSEC-CAG-NOTUM-StrepHis using Lipofectamine 2000 (Life Technologies) according to manufacturer's instructions. Supernatants from the adherent cells were harvested at 48 hours, and the recombinant His tagged protein purified on Ni-NTA H isTrap column (GE Amersham) using an AKTA prime instrument. Recombinant protein (i.e., hNotum-His) was eluted from the column using a linear gradient of imidazole (final concentration 500 mM), and the fractions containing the Notum protein pooled, concentrated, and further purified on a Superdex200 size exclusion column using an AKTA FPLC to collect monomeric protein. Purified Notum protein was confirmed by ELISA and by protein blot analysis. Collected material was used for immunization in subsequent Examples.

Similarly, His tagged murine Notum (i.e., Notum-His) was subsequently fabricated and expressed using substantially the same techniques as set forth immediately above and the murine Notum gene described in Example 8 below. This construct was also used to characterize the modulators of the present invention as described in ensuing Examples.

### Example 8

### Construction and Expression of a Fc-Notum Fusion Modulators

Additional, relatively more soluble, Notum proteins were produced for use as modulators, immunogens, assay reagents and for in vivo studies. More particularly, Fc constructs were made using human Notum and the orthologs for mouse and Rhesus macaque (Macaca mulatta or macaque), respectively. For the purposes of the instant application the Fc portion of such constructs will be human in origin unless otherwise specified.

As set forth in Example 7, the DNA encoding the mature human Notum protein was amplified by PCR to include in frame, flanking EcoR I and Nco I restriction sites, and subcloned between the EcoR I and Nco I sites of pFUSE-mIgG₂b vector (Invivogen) to generate pFUSE-NOTUM-mIgG, comprising an IL-2 signal peptide sequence, fused in frame to the sequences encoding the mature human Notum protein, fused in frame with sequences encoding the Fc domains derived from the mouse IgG2b gene. The mouse IgG2b Fc domain was replaced by a DNA sequence encoding the human IgG2 Fc, which had been amplified by PCR from the plasmid pFUSE-hIgG₂ (Invivogen). The human IgG2 Fc PCR product was digested with Bgl II and Nhe I, and subcloned into the same sites in the vector pFUSE-NOTUM-mIgG, to yield pNOTUM-hIgG₂ hFc, comprising an IL-2 signal peptide sequence, fused in frame to the sequences encoding the mature human Notum protein, fused in frame with sequences encoding the Fc domains derived from the human IgG2 gene. The amino acid sequence (SEQ ID NO: 333) and nucleic acid sequence (SEQ ID NO: 334) of an exemplary human Fc-Notum fusion construct are set forth in FIG. 1D wherein the Notum portion of the molecule is underlined.

Recombinant human Notum-Fc protein (i.e., hNotum-Fc) was produced in CHO-S cells (Life Technologies) that were transfected with pNOTUM-hIgG₂ hFc plasmid using linear poylethylenimine and standard methods (See e.g., Durocher, Y. et al. Nucleic Acids Res. (2002) 30:e9 which is incorporated herein by reference). Five days after transfection, the recombinant protein was purified from the supernatant using a Protein A columns and manufacturer's instructions (GE Amersham). Material eluted from the column was concentrated (to approximately 1 mg/mL) and the buffer exchanged to PBS.

Using similar molecular biological and DNA cloning techniques, fusion constructs comprising mouse Notum and macaque Notum and human Fc regions were fabricated for use in assay development efforts and in vivo product development. Sequences corresponding to the ORFs of Mus musculus Notum (GenBank NM_{—}175263) and Macaca mulatta Notum (GenBank XM_{—}001112829) were synthesized from oligonucleotides by GENEArt (Regensburg, Germany). The DNA encoding the mature murine Notum protein was amplified by PCR from the GENEArt supplied vector, and subcloned into the EcoR I and Nco I sites of pSCRXv003, a plasmid derived from pFUSE-mIgG2b in which the sequences encoding the mouse IgG2b Fc domain had been replaced with sequences encoding a human IgG2 Fc domain. This yielded plasmid pSCRXv3-mus-Notum which is largely similar to pNOTUM-hIgG₂ hFc with the substitution of murine Notum for human. Durocher, Y. et al. Supra

Similarly, the DNA encoding the mature M. mulatta Notum protein was amplified by PCR from the GENEArt supplied vector and subcloned into the EcoR I and Bgl II sites of pSCRXv003 to yield pSCRXv003-mac-Notum (again similar to pNOTUM-IgG₂ hFc with the substitution of macaque Notum for human). Recombinant murine and macaque Notum-human Fc tagged proteins were produced as needed in CHO-S cells as described for the human-Fc tagged human Notum, above.

### Example 9

### Generation of Anti-Notum Antibodies Using Notum Constructs

Notum modulators in the form of murine antibodies were produced in accordance with the teachings herein through inoculation with hNotum-His or hNotum-Fc. In this regard three strains of mice were used to generate high affinity, murine, monoclonal antibodies that can be used therapeutically to inhibit the action of Notum for the treatment of neoplastic disorders. Specifically, Balb/c, CD-1 and FVB mouse strains were immunized with human recombinant Notum and used to produce hybridomas as follows:

Murine antibodies were generated by immunizing 6 female mice (2 each: Balb/c, CD-1, FVB) with various preparations of Notum antigen. Immunogens included His tagged human Notum, or Notum-Fc expressed in 293 cells. Mice were immunized via footpad route for all injections. 10 μg of Notum immunogen emulsified with an equal volume of TITERMAX or alum adjuvant were used for immunization.

A solid-phase ELISA assay was used to screen mouse sera for mouse IgG antibodies specific for human Notum. Briefly, plates were coated with Notum-His (from Example 7) at different concentrations ranging from 0.01-1 μg/mL in PBS overnight. After washing with PBS containing 0.02% (v/v) Tween 20, the wells were blocked with 3% (w/v) BSA in PBS, 200 μL/well for 1 hour at RT. Mouse serum dilutions were incubated on the Notum-His coated plates at 50 μL/well at RT for 1 hour. The plates are washed and then incubated with 50 μL/well HRP-labeled goat anti-mouse IgG diluted 1:10,000 in 3% BSA-PBS for 1 hour at RT. The plates were washed and 100 μL/well of the TMB substrate solution was added for 15 minutes at RT. After washing, the plates were developed with TMB substrate (Thermo Scientific 34028) and analyzed by spectrophotometer at OD 450.

Sera positive immunized mice were sacrificed and draining lymph nodes (popliteal and inguinal, if enlarged) were dissected out and used as a source for antibody producing cells. Single cell suspension of B cells (6.35×10⁷ cells) were fused with non-secreting P3x63Ag8.653 myeloma cells (ATCC #CRL-1580) at a ratio of 1:1 by Electro-fusion. Electro cell fusion was performed using a fusion generator, model ECM2001, (Genetronic, Inc.). Cells were resuspended in hybridoma selection medium supplemented with HAT (Sigma #A9666) (DMEM (Cellgro cat#15-017-CM) medium containing, 15% Fetal Clone I serum (Hyclone), 1 mM sodium pyruvate, 4 mM L-glutamine, 10 μg/mL gentamicin, 50 μM 2-mercaptoethanol, 100 hypoxanthine, 0.4 μM aminopterin, and 16 μM thymidine) and then plated at 200 μL/well in twenty 96-well flat bottom tissue culture plates, based on a final plating of 2×10⁶ B cells per 96-well plate. The plates are then placed in a humidified 37° C. incubator containing 5% CO₂ and 95% air for 7-10 days.

Growth positive hybridomas wells secreting mouse immunoglobulins were screened for Notum specificity using an ELISA assay similar to that described above. Briefly, 96 well plates (VWR, 610744) were coated with 0.4 μg/mL human Notum-His in sodium carbonate buffer overnight at 4° C. The plates were washed and blocked with 1% BSA-PBS for one hour at 37° C. and used immediately or kept at 4° C. Undiluted hybridoma supernatants were incubated on the plates for one hour at RT. The plates are washed and probed with HRP labeled goat anti-mouse IgG diluted 1:10,000 in 1% BSA-PBS for one hour at RT. The plates are then incubated with substrate solution as described above and read at OD 450.

Alternatively, ELISA plates were coated with goat anti-human IgG Fc, to capture hNotum-Fc to ELISA plate. The plates were washed and blocked with 3% BSA-PBS for one hour at RT, and used to screen undiluted hybridoma supernatants. Subsequently, the plates were washed and probed with HRP labeled goat anti-mouse IgG diluted 1:10,000 in 3% BSA-PBS for one hour at RT. The plates were then incubated with substrate solution as described above and read at OD 450.

Notum specific hybridomas were expanded in cell culture were re-plated, rescreened and serially subcloned by limiting dilution, or single cell FACS sorting. The resulting clonal populations were expanded and cryopreserved in freezing medium (90% FBS, 10% DMSO) and stored in liquid nitrogen.

ELISA analysis confirmed that purified antibody from most or all of these hybridomas bind Notum in a concentration-dependent manner. It should be noted that binding Notum directly to the ELISA plate can cause denaturation of the protein and the apparent binding affinities cannot be reflective of binding to undenatured protein.

Two fusions were performed and each fusion was seeded in 20 plates (1920 wells/fusion). This yielded several dozen murine antibodies specific for human Notum.

### Example 10

### Characterization of Notum Modulators

The Notum modulators produced in the previous Example were characterized as follows:

Binding characteristics for antibodies were assessed using antibody capture Biacore technology. Disassociation constant values K_{d} (k_{off}/kₒₙ) were determined for selected antibodies. A Biacore 3000 (GE Healthcare) biosensor was used for surface plasmon resonance (SPR) kinetic measurements. Using purified antibody quantitative k_{off} constants were derived through capture the antibody on the sensor surface. Anti-mouse IgG was immobilized on the CM5 surface of sensor chip using standard amine coupling chemistry. Each mAb was captured onto an anti-IgG surface before the antigen was injected over the immobilized antibody allowing the antibody-antigen interaction to be analyzed.

Quantitative K_{d} values obtained using Biacore analysis of the anti-Notum antibodies reveals that several of the monoclonal antibodies are very high affinity with IQ measurements in the range of 1×10⁻⁷M to 7×10⁻¹⁰M.

### Example 11

### Epitope Determination of Notum Modulators

Multiplexed competitive antibody binning is outlined in the Jia et al., 2004, PMID: 15183088 which is incorporated herein by reference. Multiplexing Luminex beads were coupled with an anti-mouse IgG to capture a reference mAb. Each bead had a unique spectral coding such that each mAb was associated with a unique spectral address. All of the mAb bead complexes were pooled into a master mix and aliquoted into individual wells of 96-well micro titer plates. The master mix of reference antibody-bead complexes in each well was incubated first with antigen, then with a probe mAb, one different probe mAb per well. The antigen in the competitive antibody binning assay was recombinant Notum-His. The probe mAbs only bound to antigen that had been captured by a reference mAb that recognized a different epitope. The signal was read as RFU on a Luminex 100. This experiment showed the screened antibodies bound to at least four different epitopes on the Notum protein.

### Example 12

### Sequencing of Notum Modulators

Based on the foregoing, a number of exemplary distinct monoclonal antibodies that bind immobilized human Notum with apparently high affinity were selected. As shown in a tabular fashion in FIGS. 7A and 7B, sequence analysis of the DNA encoding mAbs from Example 9 confirmed that many had a unique VDJ rearrangements and displayed novel complementarity determining regions. Note that the complementarity determining regions set forth in FIG. 7B are defined as per Chothia et al., supra

For initiation of sequencing TRIZOL reagent was purchased from Invitrogen (Life Technologies). One step RT PCR kit and QIAquick PCR Purification Kit were purchased from Qiagen, Inc. with RNasin were from Promega. Custom oligonucleotides were purchased from Integrated DNA Technologies.

Hybridoma cells were lysed in TRIZOL reagent for RNA preparation. Between 10⁴ μL and 10⁵ cells were resuspended in 1 ml TRIZOL. Tubes were shaken vigorously after addition of 200 μl of chloroform. Samples were centrifuged at 4° C. for 10 minutes. The aqueous phase was transferred to a fresh microfuge tube and an equal volume of isopropanol was added. Tubes were shaken vigorously and allowed to incubate at room temperature for 10 minutes. Samples were then centrifuged at 4° C. for 10 minutes. The pellets were washed once with 1 ml of 70% ethanol and dried briefly at room temperature. The RNA pellets were resuspended with 40 μl of DEPC-treated water. The quality of the RNA preparations was determined by fractionating 3 μL in a 1% agarose gel. The RNA was stored in a −80° C. freezer until used.

The variable DNA sequences of the hybridoma amplified with consensus primer sets specific for murine immunoglobulin heavy chains and kappa light chains were obtained using a mix of variable domain primers. One step RT-PCR kit was used to amplify the VH and VK gene segments from each RNA sample. The Qiagen One-Step RT-PCR Kit provides a blend of Sensiscript and Omniscript Reverse Transcriptases, HotStarTaq DNA Polymerase, Qiagen OneStep RT-PCR Buffer, a dNTP mix, and Q-Solution, a novel additive that enables efficient amplification of “difficult” (e.g., GC-rich) templates.

Reaction mixtures were prepared that included 3 μL of RNA, 0.5 of 100 μM of either heavy chain or kappa light chain primers 5 μL of 5×RT-PCR buffer, 1 μL dNTPs, 1 μL of enzyme mix containing reverse transcriptase and DNA polymerase, and 0.4 μL, of ribonuclease inhibitor RNasin (1 unit). The reaction mixture contains all of the reagents required for both reverse transcription and PCR. The thermal cycler program was RT step 50° C. for 30 minutes 95° C. for 15 minutes followed by 30 cycles of (95° C. for 30 seconds, 48° C. for 30 seconds, 72° C. for 1.0 minutes). There was then a final incubation at 72° C. for 10 minutes.

To prepare the PCR products for direct DNA sequencing, they were purified using the QIAquick™ PCR Purification Kit according to the manufacturer's protocol. The DNA was eluted from the spin column using 50 μL of sterile water and then sequenced directly from both strands. PCR fragments were sequenced directly and DNA sequences were analyzed using VBASE2 (Retter et al., Nucleic Acid Res. 33; 671-674, 2005).

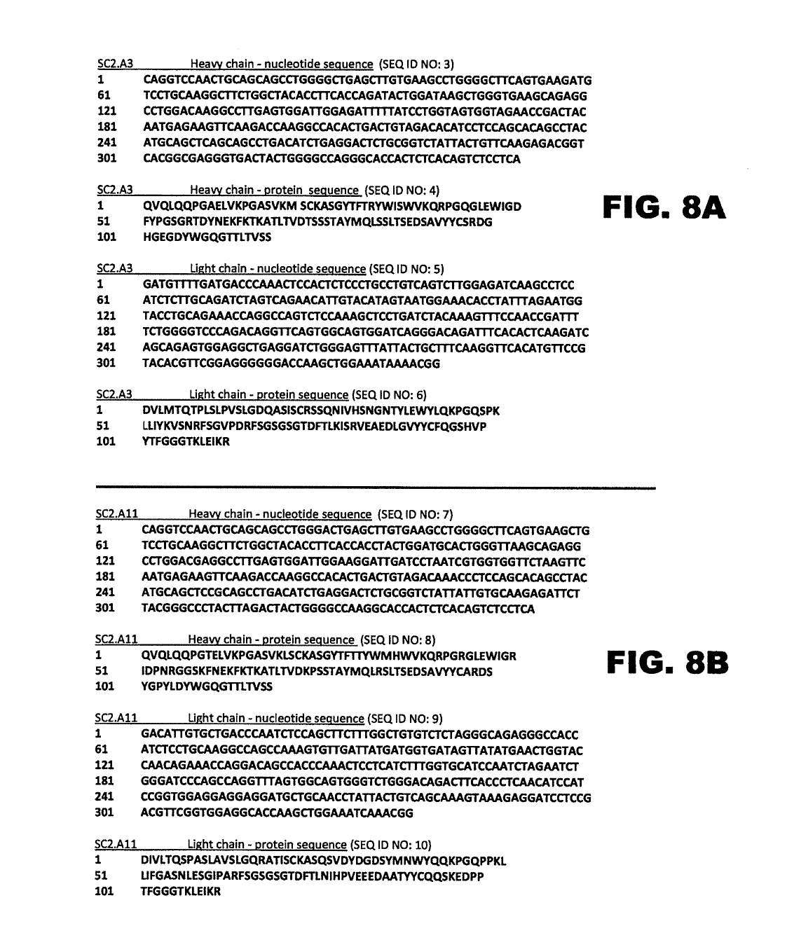

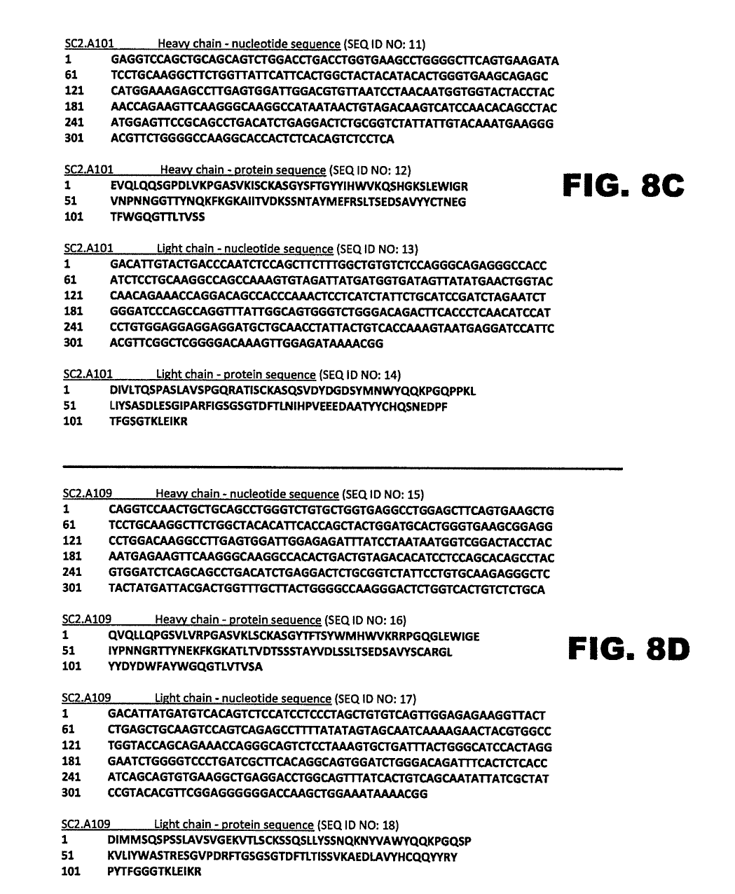

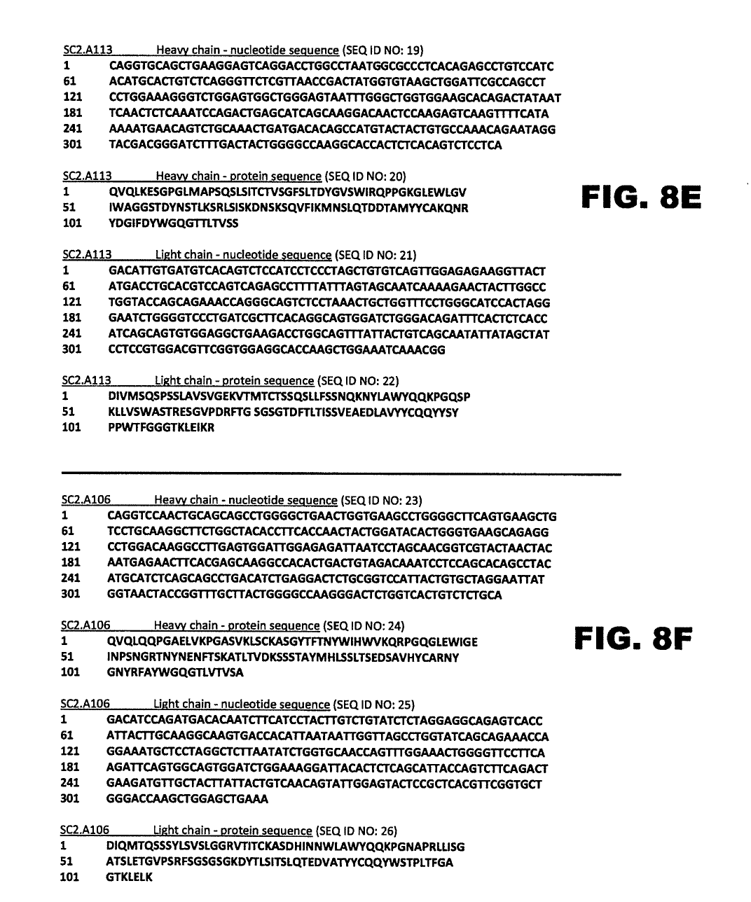

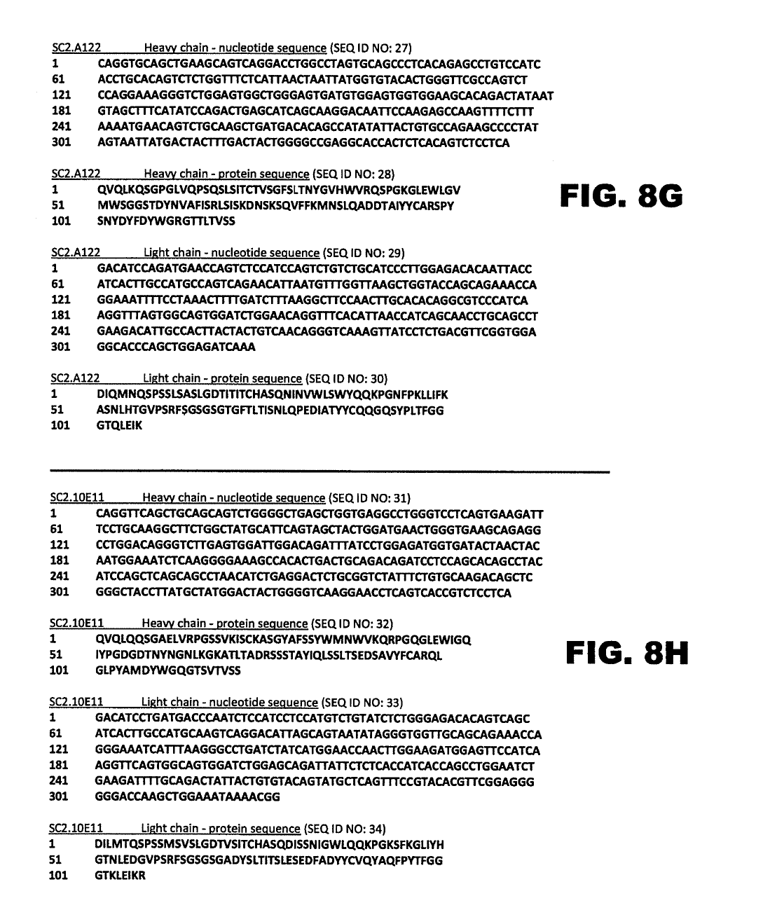

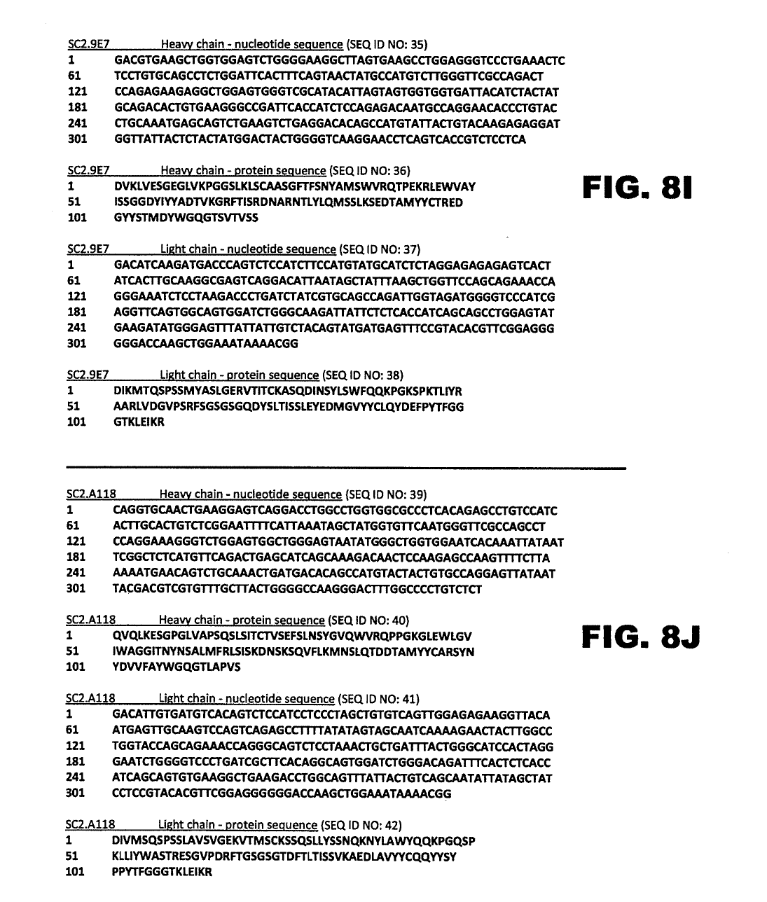

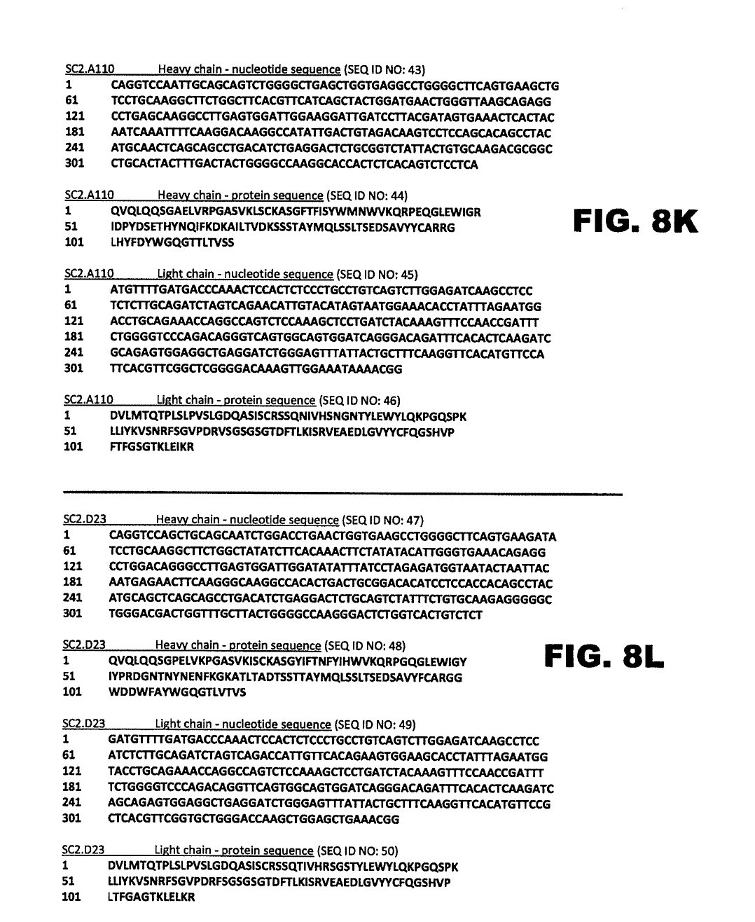

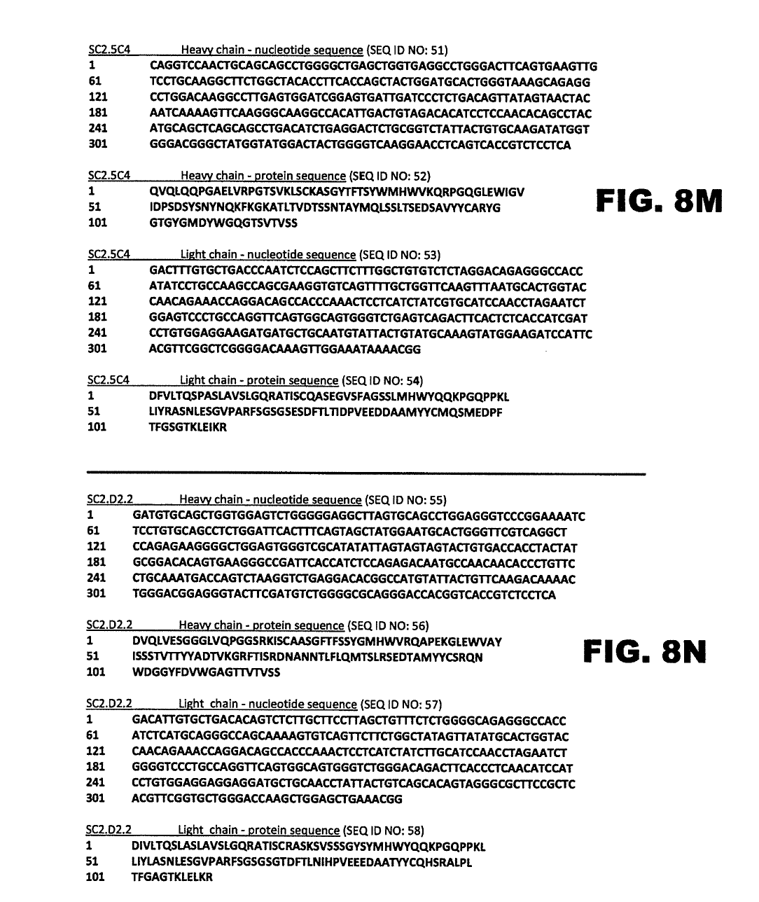

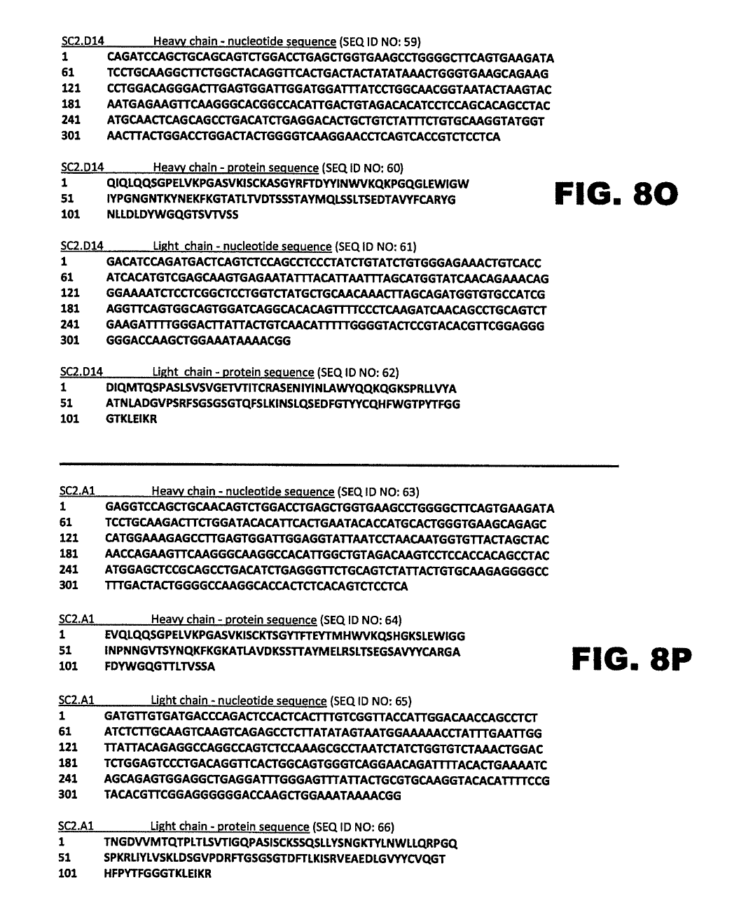

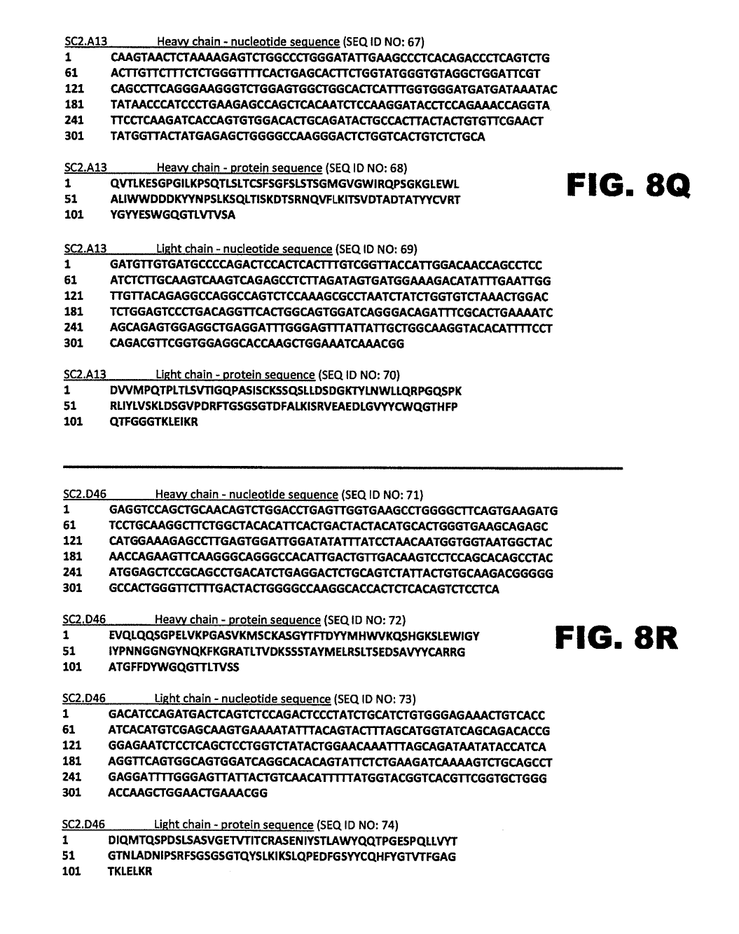

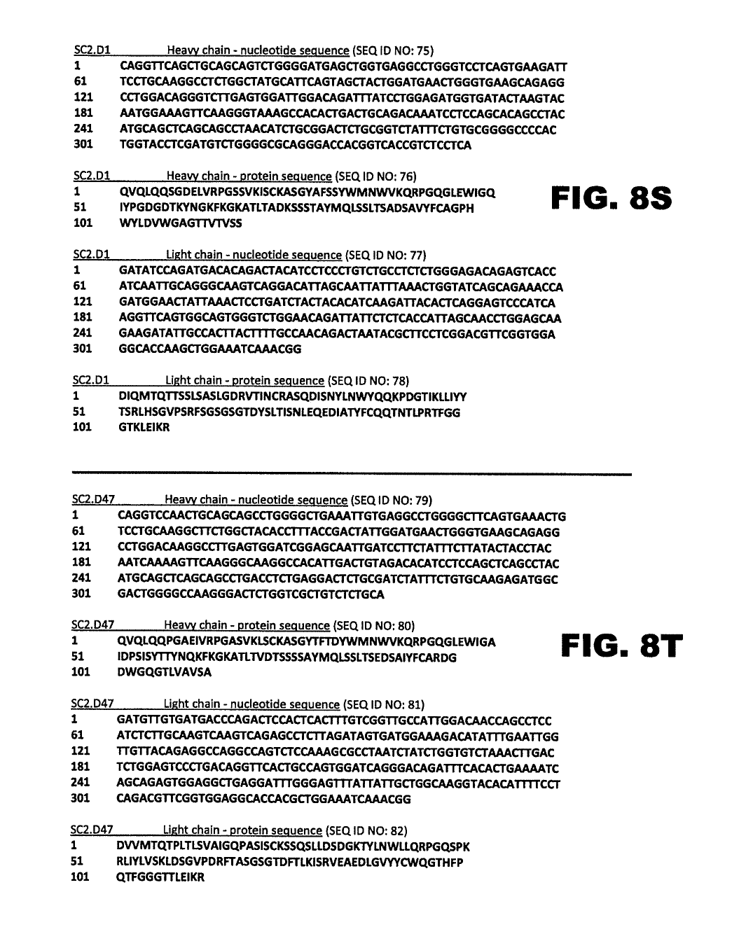

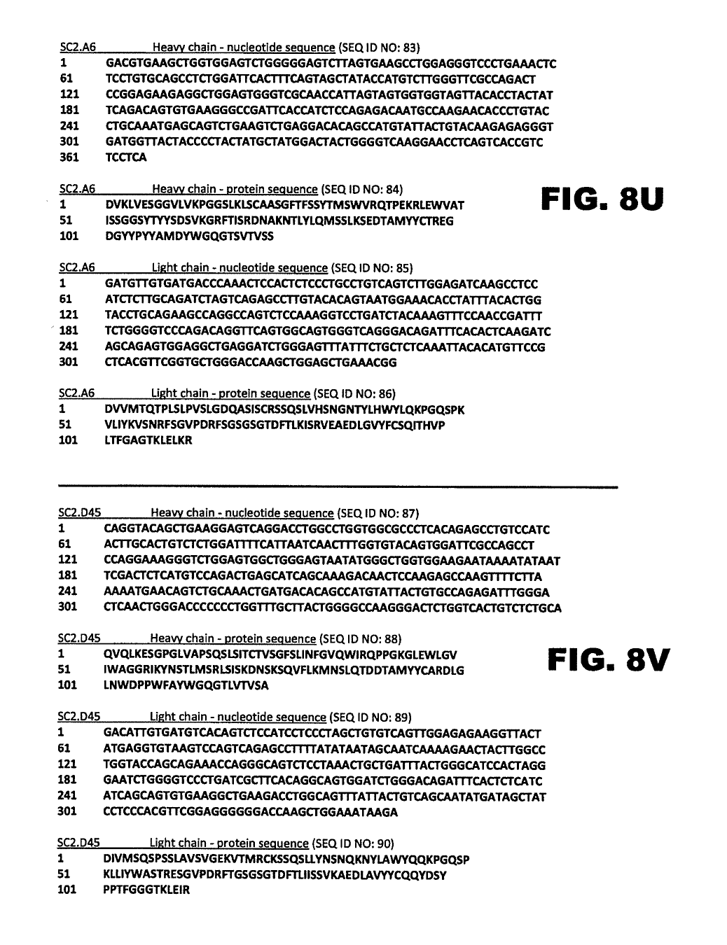

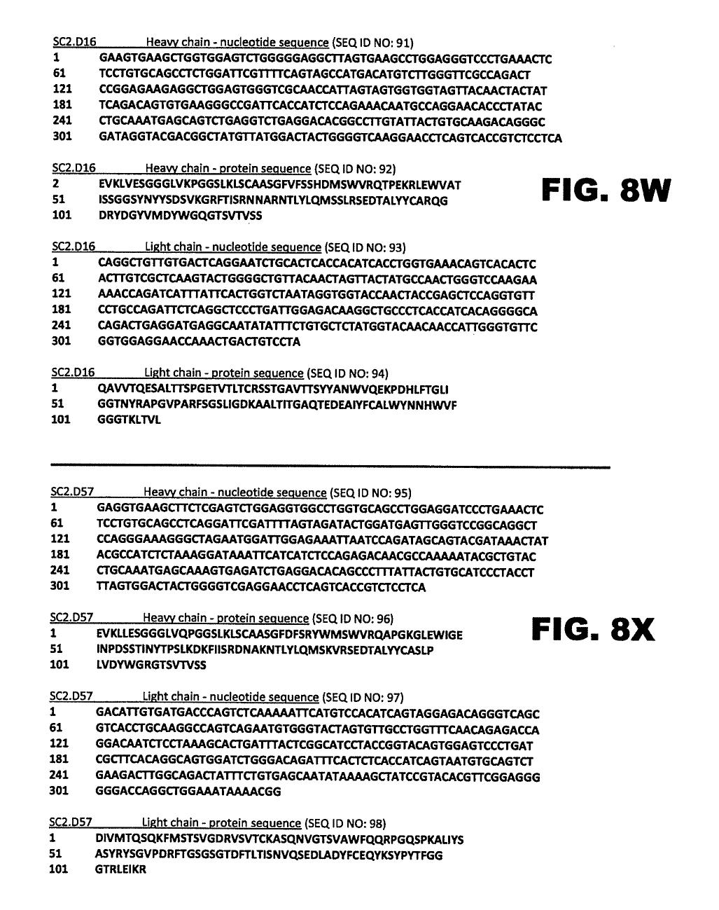

As discussed above the amino acid and nucleic acid sequences for twenty-four (24) exemplary antibody heavy and light chain variable regions are set forth in FIGS. 8A-8X respectively (SEQ ID NOs: 3-98) while the genetic arrangements and derived CDRs (as defined by Chothia et al., supra) of these and additional anti-hNotum antibodies are set forth, respectively, in a tabular form in FIGS. 7A and 7B (SEQ ID NOs: 103-330).

### Example 13

### Construction of Notum Modulators Comprising Point Mutations

As previously discussed, Notum is a member of the α/β hydrolase superfamily of enzymes. Sequence analysis of Notum identifies a signature catalytic elbow sequence of GXSXG, beginning at Gly230, and which Ser232 would be the putative nucleophilic residue of the catalytic triad of nucleophile, acidic residue and histidine characteristic of this superfamily. Site directed mutagenesis of the orthologous residue in the Drosophila (S237A, Kreuger, 2004, PMID: 15469839) and murine (S239A, Traister, 2008, supra) forms leads to an inactive protein; therefore, standard molecular biological techniques (Quick Change Mutagenesis Kit, Stratagene/Agilent, Inc.) were used to perform site directed mutagenesis on the wild-type human Notum protein to generate the S232A mutation in the His tagged version of the protein (i.e., hNotum-S232A-His). Similarly, sequence alignments suggest that human D340 is the catalytic acidic residue; therefore, this residue was changed using the same kit to generate a D340A mutated version of the molecule. As set forth in Examples 7 and 8, PCR cloning was used to clone the Notum domain containing this mutation into the human Notum-hFc expression vector (i.e., hNotum-S232A-hFc). The constructs were then expressed and purified as set forth above.

### Example 14

### Notum Modulators Alter Wnt3A Canonical Signaling

Drosophila Notum has been shown to be a functional antagonist of Wingless signaling, while murine Notum has been shown to antagonize the induction of a beta-catenin luciferase reporter in transient transfection assays.

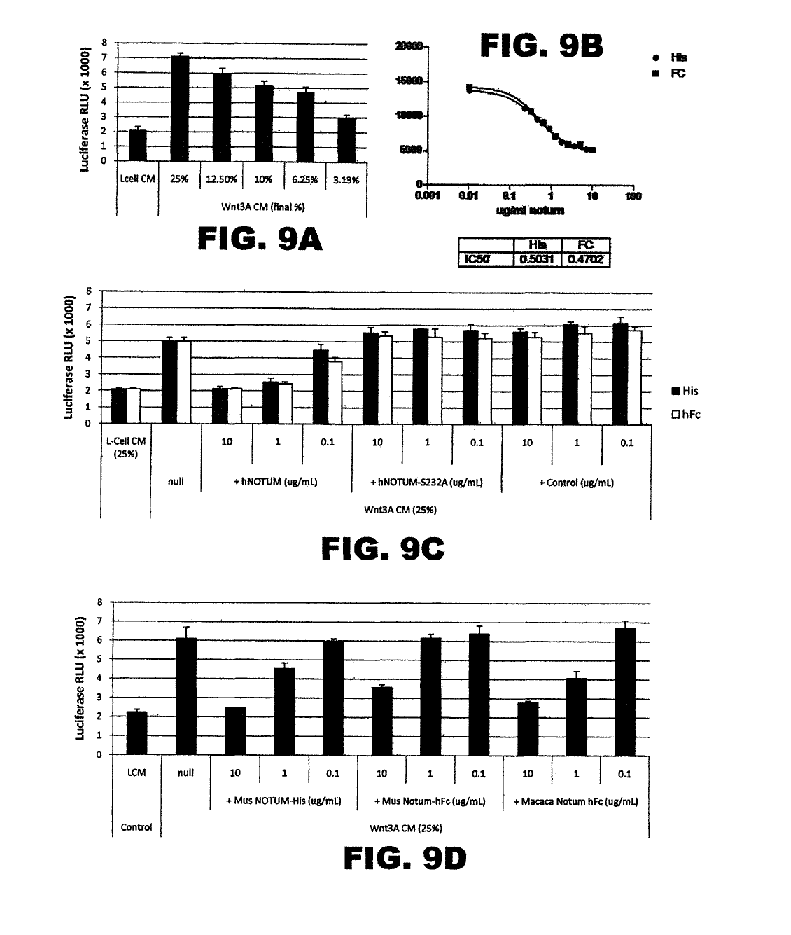

To generate a stable population of cells that contain a reporter for the activation of canonical Wnt signaling, HEK 293T cells were transduced with a lentiviral vector, pGreenFirel-TCF (System Biosciences) which encodes a bifunctional GFP and luciferase reporter cassette under the control of a minimal CMV reporter linked to four tandem repeats of the transcriptional response elements for TCF. Transduced cells populations, termed 293.TCF cells, were subsequently used in a Wnt3A canonical signaling assay as follows: 2.5×10⁴ 293.TCF cells were plated per well of a 96-well tissue culture plate in 50 μL of serum-free DME medium. After 24 hours of serum starvation, 25 μL of various dilutions of conditioned medium (CM) from L/Wnt3A cells (ATCC CRL-2647; Willert, 2003) or undiluted CM from parental L-cells (ATCC CRL-2648) along with 25 μL of DMEM+0.2% FBS were added to each well. Eighteen hours after addition of CM, 100 μL of One-Glo solution (ProMega Corp.) was added to each well. The contents of each well were then mixed thoroughly to lyse the cells, 100 μL of lysate transferred to black 96-well plates, and the luminescence in each well read after 5 mins using a Wallac Victor3 Multilabel Counter (Perkin-Elmer Corp). As can be seen in FIG. 9A, the cells exposed to differing concentrations of CM containing Wnt3A typically showed between 2 and 4-fold induction of luciferase signal relative to cells exposed to L-cell control CM. More particularly, as the Wnt3A+ CM media is diluted from 25% down to approximately 3%, activation of the Wnt pathway is reduced with a corresponding decrease in luminescence.

Once the luciferase reporter system was established, assays for determining the bioactivity of various Notum modulators were performed as follows. 2.5×10⁴ 293.TCF cells were plated per well of a 96-well tissue culture plate in 50 μL of serum-free medium. After 23 hours of serum starvation, 25 μL of DMEM+0.2% FBS containing various Notum modulators at various concentrations (e.g., hNotum-His, hNotum-hFc, hNotum-S232A-His, murine Notum-His, murine Notum-hFc, macaque Notum-hFc, control protein-His or control protein-hFc obtained as per Examples 7, 8 and 13 above), were added to the cells. After 1 hour, 25 μL of Wnt3A or control L-cell CM were added to each well. Eighteen hours after addition of CM, 100 μL of One-Glo solution (ProMega Corp.) was added to each well, the contents of each well mixed thoroughly to lyse the cells, 100 μL of lysate transferred to black 96-well plates, and the luminescence read after 5 minutes.

As can be seen in FIGS. 9B, 9C and 9D human Notum-His, human Notum-hFc, murine Notum-His, murine Notum-hFc, and macaque Notum-hFc all functionally antagonize Wnt3A-mediated induction of luciferase in the 293.TCF cells, whereas the human-NOTUM S232A mutant from Example 13 (His and hFc) and the control-His and control-hFc proteins did not antagonize Wnt3A-mediated induction of luciferase in the 293.TCF cells.

Besides demonstrating the development of a functional assay useful for characterizing compounds of the instant invention, FIGS. 9B-9D show that both soluble His tagged Notum constructs and Fc-Notum fusion proteins act effectively as Notum modulators in accordance with the teachings herein. More specifically, FIG. 9B illustrates the concentration dependent effect of hNotum-Fc and hNotum-His modulators on the Wnt pathway as shown by a decrease in luciferase activity with a calculated IC50 of 0.4702 and 0.5031 respectively. These results are confirmed in FIG. 9C which graphically illustrate that Notum-hFc and Notum-His modulators antagonize the Wnt3A pathway in a concentration dependent manner while the mutant Notum modulators of Example 13 do not. Similarly, FIG. 9D shows that murine Notum modulators (His and Fc) and macaque Notum-hFc also antagonize the Wnt3A canonical pathway in a concentration dependent manner. The foregoing data validates the Notum I Wnt bioassay and shows that various soluble Notum constructs comprising at least a portion of the Notum extracellular domain can antagonize the Wnt pathway.

### Example 15

### Notum Modulators Neutralize Notum Activity in Vitro

Using the 293.TCF cells described above, supernatants from hybridomas and/or purified antibodies shown to bind Notum by ELISA assays (Example 9) were screened for their ability to neutralize hNotum-His or hNotum-Fc activity as follows. 2.5×10⁴ 293.TCF cells were plated per well of a 96-well tissue culture plate in 50 μL of serum-free medium. After 23 hours of serum starvation, 10 μL of DMEM+0.2% FBS containing various Notum proteins at various concentrations were mixed with either 15 μL of supernatant from the hybridoma, or 15 μL of purified antibody at various concentrations, and allowed to incubate for 5 minutes at room temperature. The 25 μL antibody:Notum mixture was then added to the 293.TCF cells. After 1 hour, 25 μL of Wnt3A or control L-cell CM were added to each well. Eighteen hours after addition of CM, 100 μL of One-Glo solution (ProMega Corp.) was added to each well. The contents of each well were then mixed thoroughly to lyse the cells, 100 μL of lysate transferred to black 96-well plates, and the luminescence read after 5 minutes. For analysis of antibody activity, either RAW luciferase RLU were plotted, or the data was normalized to set Wnt3A CM activity at 1 and L-cell control medium at zero (graphed as Normalized Wnt3-induced luciferase activity), or normalized to set Wnt3A CM activity at 1 and the luciferase signal at maximal Notum antagonist activity as zero (graphed as % neutralizing activity).

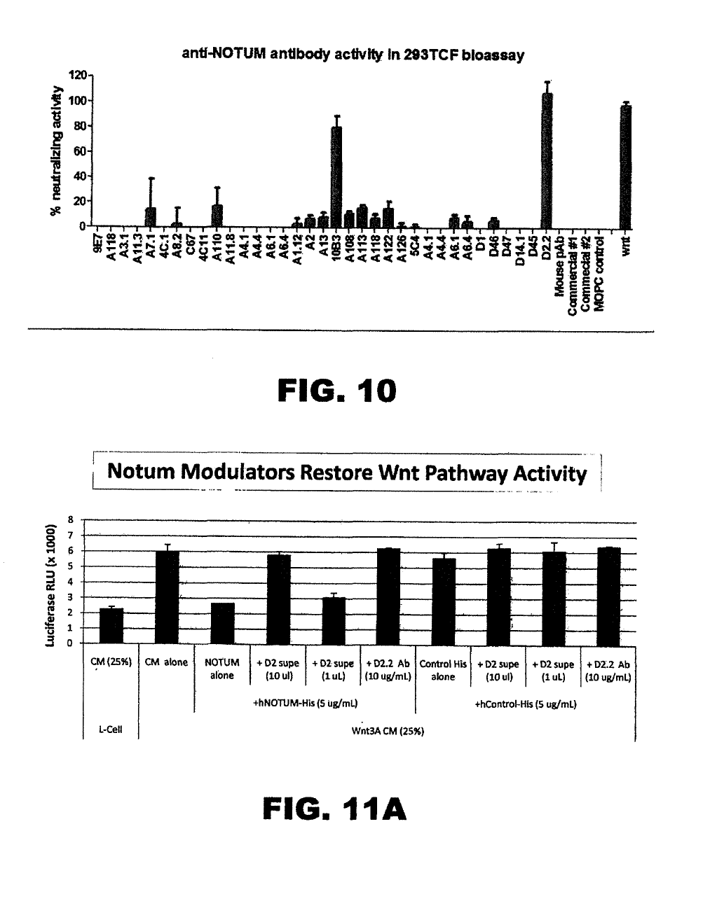

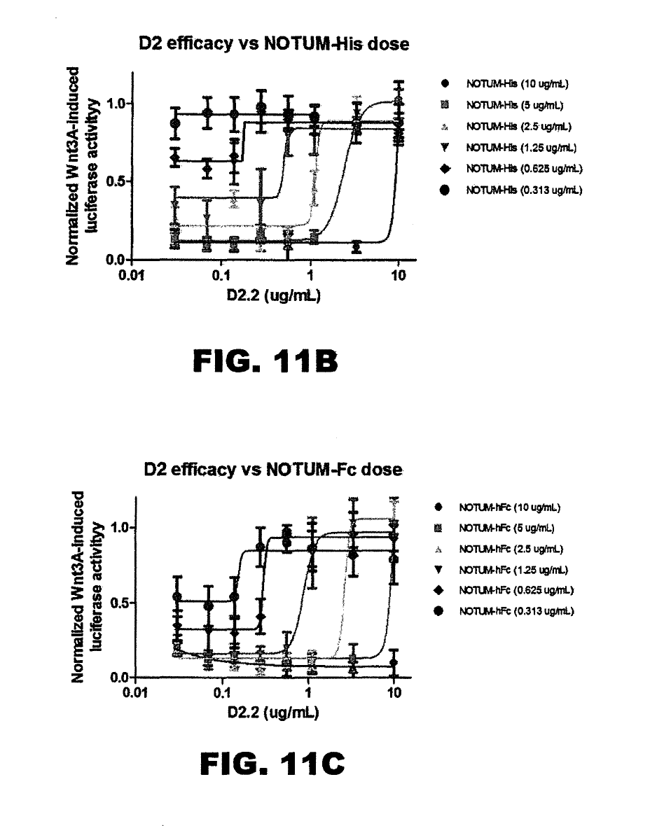

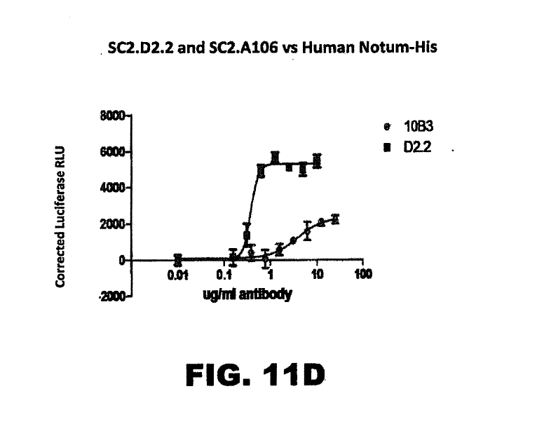

As can be seen in FIG. 10, several of the antibodies were able to inhibit Notum activity when added at a concentration of 10 μg/mL. Moreover, selected Notum modulators (e.g., SC2.A106 [aka 10B3] and SC2.D2.2) proved to be particularly effective and showed Notum inhibition of greater than 80% at the same concentration. Antibody SC2.D2.2 was further characterized to demonstrate its ability to inhibit the activity of human Notum in the 293.TCF luciferase induction assay, restoring the luciferase signal to near the same levels as negative controls (FIG. 11A). More particularly, FIG. 11A shows that SC2.D2.2 supernatant and purified antibody acts in a concentration dependent manner to antagonize the effects of added hNotum-His. This effect is further illustrated in FIGS. 11B and 11C wherein SC2.D2.2 purified antibody is titrated against various concentrations of Notum-His (FIG. 11B) and Notum-hFc (FIG. 11C) respectively. The inflection points in the resulting curves in each FIG. confirm that the modulation activities of the antibody act in a concentration dependent manner to antagonize Notum activity relative to the absolute amount of soluble Notum. Moreover, as seen in FIG. 11D a second Notum modulator, SC2.A106, was also able to inhibit the activity of human Notum-His although apparently not to the same extent as SC2.D2.2. Taken together these results show that the Notum modulators disclosed herein provide effective neutralization candidates and are strongly indicative of the use of such compounds to reduce tumor initiating cell frequency.

### Example 16

### ELISA Characterization of Notum Modulators

The high degree of specificity displayed by antibodies often results in varying potencies against antigen orthologs, which can affect the efficacy of these molecules in different animal models of disease. To investigate structure-function relationships of Notum, cDNA sequences that encode the Notum protein of the human, macaque and mouse (Examples 7 and 8) were cloned. Deduced amino acid sequences of the Notum proteins from these animals, showed a high degree of homology, which explains the biologic and immunological cross-reactivity that has been observed in a number of species. As previously discussed, human Notum is 97% identical to monkey Notum, and 91% to mouse. There is a full conservation of the (1) disulfide bonds (sixteen Cys residues in the mature human Notum sequence are conserved in the mouse Notum sequence) (2) N-glycosylation sites; and (3) predicted active domain based on the common enzymatic activity. Most of the amino acid replacements are conservative. The N-terminal part of the human and mouse sequence showed the most variation, with several amino acid substitutions, deletions, and/or insertions (FIG. 1C).

As per Example 9 human Notum antigen constructs were used to immunize mice and produce the modulators. With 91% sequence homology between human and mouse Notum protein it was expected that most of these antibodies cross react with the mouse Notum protein.

Binding of selected hybridoma derived mouse mAbs to purified Notum antigens generated from transient transfection of human and mouse Notum cDNAs was tested using ELISA assay. Human and mouse Notum were used to directly coat ELISA plate using art recognized techniques. Binding of mouse mAbs, was detected with HRP-conjugated goat anti mouse antibody and followed by colorimetric horseradish proxidase substrate (TMB substrate, Thermo Scientific). The absorbance of each well of the ELISA plates was measured at 450 nm on a microplate reader.

As seen in TABLE 1 immediately below, twenty-two of forty-six antibodies tested were specific for the human Notum:

TABLE 1

| Human Specific | Human/Mouse cross reactive |
|---|---|
| SC2.A3 | SC2.A1 |
| SC2.A5 | SC2.A2 |
| SC2.A7 | SC2.A6 |
| SC2.A10 | SC2.A8 |
| SC2.A11 | SC2.A13 |
| SC2.A12 | SC2.A101 |
| SC2.A19 | SC2.A109 |
| SC2.A110 | SC2.6C1 |
| SC2.A184 | SC2.A118 |
| SC2.D2.2 | SC2.A113 |
| SC2.D31 | SC2.10E11 |
| SC2.D3 | SC2.4F4 |
| SC2.D9 | SC2.4D4 |
| SC2.D17 | SC2.A106 (aka 10B3) |
| SC2.D19 | SC2.D14 |
| SC2.D22 | SC2.D16 |
| SC2.D30 | SC2.D23 |
| SC2.D35 | SC2.D34 |
| SC2.D41 | SC2.D44 |
| SC2.D49 | SC2.D45 |
| SC2.D51 | SC2.D16 |
| SC2.D53 | SC2.D34 |
| | SC2.D54 |
| | SC2.D57 |

### Example 17

### Epitope Mapping of SC2.D2.2 Notum Modulator

To better understand the structural basis for the interaction of SC2.D2.2 with human Notum, a chimeric Notum protein was fabricated. This approach takes advantage of the fact that the orthologs are structurally related. To that end a chimeric Notum molecule composed of the N terminal of the human mature Notum protein (residues 19-144) fused to the mouse Notum (mouse residues 150-484) (genes both consistent with Example 7) was generated and expressed in a similar manner to that set forth in previous Examples. The BamHI restriction cleavage site in human Notum gene was used for construction of in-frame fusion Notum chimeric protein. An expression vector was then constructed containing the His tagged chimeric Notum sequence. Chimeric Notum molecule was tested and found to be functionally active in the Wnt bioassay described above (see Example 27 below).

Binding of SC2.D2.2 and other mouse mAbs to purified Notum molecules generated from transient transfection of Human and Mouse Notum cDNAs were tested using ELISA assay with human Notum, mouse Notum and chimeric human/mouse Notum coated directly on ELISA plate. Binding of anti-Notum mAbs was detected with HRP-conjugated goat anti mouse antibody and followed by colorimetric horseradish peroxidase substrate (TMB substrate Thermo Scientific). The absorbance of each well of the ELISA plates was measured at 450 nm on a microplate autoreader.

The aforementioned ELISA assay confirmed the binding of the SC2.D2.2 antibody to human Notum and to the Notum chimeric protein, confirming that the SC2.D2.2 epitope is within the first 135 residues of the N terminus of the human Notum protein.

### Example 18

### Notum Modulators Exhibit Differential Species Activity

Using the 293.TCF cells, purified SC2.D2.2 and SC2.A106 antibodies were tested for their ability to neutralize murine Notum-His or macaque Notum-Fc activity as follows. 2.5×10⁴ 293.TCF cells were plated per well of a 96-well tissue culture plate in 50 μL of serum-free medium. After 23 hours of serum starvation, 10 μL of DMEM+0.2% FBS containing the Notum proteins at various concentrations were mixed with 15 μL of purified antibody at various concentrations, and allowed to incubate for 5 minutes at room temperature. The 25 μL antibody/Notum mixture was then added to the 293.TCF cells. After 1 hour, 25 μL of Wnt3A or control L-cell CM were added to each well. Eighteen hours after addition of CM, 100 μL of One-Glo solution (ProMega Corp.) was added to each well. The contents of each well were mixed thoroughly to lyse the cells, 100 μL of lysate transferred to black 96-well plates, and the luminescence read after 5 minutes.

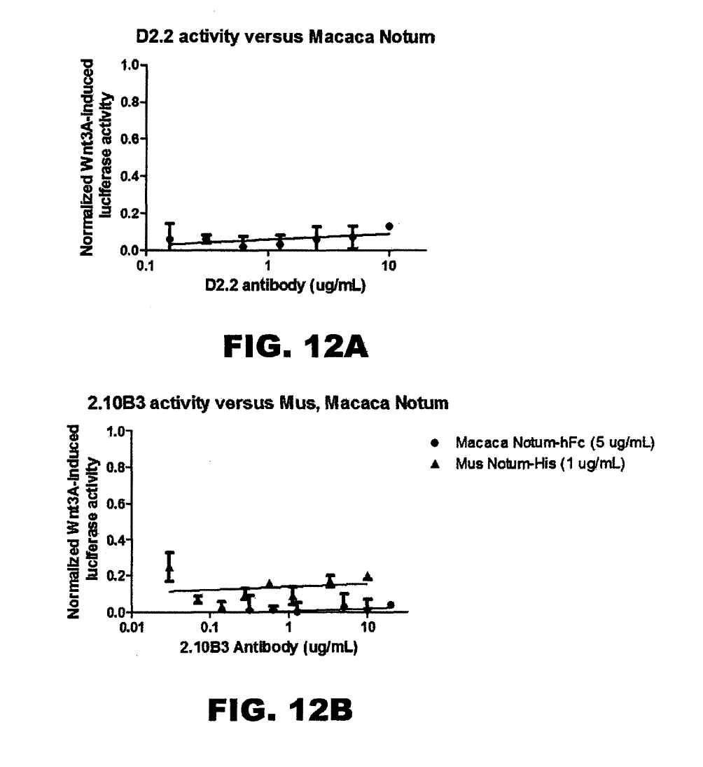

In addition to not being cross reactive with murine Notum as seen in Example 16, SC2.D2.2 did not inhibit the activity of either murine Notum or macaque Notum (FIG. 12A). Similarly, the antibody SC2.A106 did not inhibit the activity of murine or macaque Notum (FIG. 12B) despite showing cross reactivity with murine Notum in Example 16.

In accordance with the ELISA data in Example 17 suggesting that the epitope was in the first 135 amino acid residues of the N-terminus of the mature Notum protein, and the inability of SC2.D2.2 to inhibit the function of macaque Notum or bind or inhibit the function of murine Notum, the binding of SC2.D2.2 is likely to interfere with Asn129 (as numbered from the start of the mature Notum protein) activity. See the sequence alignment in FIG. 1C. That is, as the only amino acid difference in the relevant portion of the macaque and human Notum is at Asn129, interference with this site, either by direct occlusion (i.e. the epitope comprises the epitope) or conformational changes or steric hindrance is strongly suggested.

### Example 19

### Notum Modulators Reduce Notum Antagonism of the Wnt Pathway in a Co-Culture Assay

In order to more closely model the behavior of Notum producing cells in vivo, co-culture experiments were performed in which effector cells, either parental 293T cells (293.null) or 293T cells expressing soluble Notum (i.e., 293.Notum cells), were mixed in varying ratios with reporter 293.TCF cells. Notum activity or inhibition in the presence of antibodies was then determined from these cell mixtures after treatment with Wnt3A CM. Briefly, three different ratios of effector to reporter cells were tested: 2:1, 1:1 and 1:2.5, corresponding to 5×10⁴: 2.5×10⁴, 2.5×10⁴: 2.5×10⁴ cells, or 2.5×10⁴1.0×10⁴ cells per well of a 96-well plate by mixing the cells in 50 μL serum free medium per well prior to plating.

For direct co-culture experiments, after 23 hours of serum starvation 25 μL of Wnt3A or control L-cell CM were added to each well along with 25 μL of DMEM+0.2% FBS per well to a final volume of 100 μL. Eighteen hours after addition of CM, 100 μL of One-Glo solution (ProMega Corp.) was added to each well. The contents of each well were then mixed thoroughly to lyse the cells, 100 μL of lysate transferred to black 96-well plates, and the luminescence read after 5 minutes.

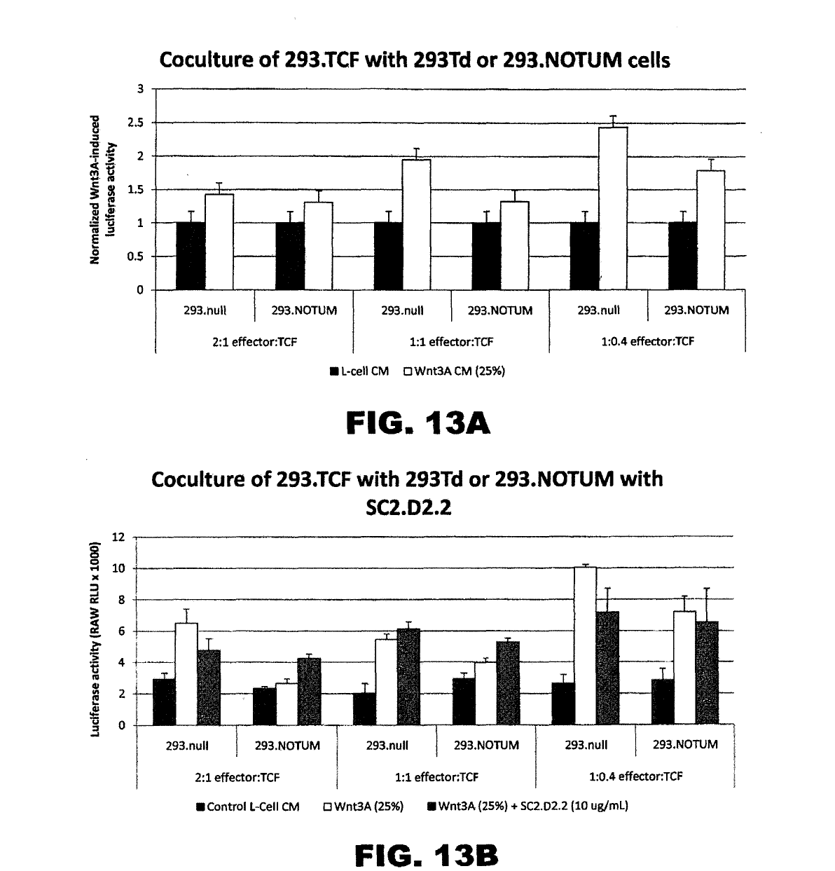

As can be seen in FIG. 13A, in all instances, co-culture with effector cells secreting Notum leads to lower levels of Wnt3A-induced luciferase activity versus co-culture with parental 293T effector cells at all ratios. Interestingly, the overall induction of luciferase activity increases as the total number of cells per well decreases, suggesting either media exhaustion effects or possibly effects due to a low level of secreted Notum from the parental 293 cells themselves.

For the antibody antagonism experiments, the mixture of cells was plated into wells containing 25 μL of DMEM+0.2% FBS and antibody at a final concentration of 10 μg/mL. Twenty-three hours after plating, 25 μL of Wnt3A or control L-cell CM were added to each well. Eighteen hours after addition of CM, 100 μL of One-Glo solution (ProMega Corp.) was added to each well. The contents of each well were then mixed thoroughly to lyse the cells, 100 μL of lysate transferred to black 96-well plates, and the luminescence read after 5 minutes.

As can be seen in FIG. 13B, addition of SC2.D2.2 to the co-cultures of 293.null and 293.TCF cells has little effect on the induction of luciferase activity by Wnt3A CM. In the case of the co-cultures of 293.Notum to 293.TCF cells, addition of the SC2.D2.2 antibody increases the amount of Wnt3A-induced luciferase, consistent with antibody inhibiting the Notum being secreted from the 293-Notum cells, and blocking its paracrine effects on the 293.TCF cells. Such results in an experimental system that more closely mimics in vivo conditions (e.g. an autocrine or paracrine effect of Notum), suggests that the Notum modulators disclosed herein can effectively influence Notum mediated events in animals.

### Example 20

### Detection of Notum Protein in Cell Lysates

In an attempt to identify mouse monoclonal antibodies that detect protein expression by Western blot and, potentially, immunohistochemistry, protein cell lysates from four different cell lines (HepG2, SW480, K562 and CHO) were run on NuPAGE 4-12% Bis-tris gels (Life Technologies) under denaturing conditions using art standard techniques. The protein was then transferred to PVDF membrane using the iBlot® Dry Blotting System (Life Technologies) according to the manufacturer protocol and membranes were blocked with 3% BSA in PBST for two hours. After probing the membrane with 1 μg/mL of either murine polyclonal, or one of two murine monoclonal antibodies (SC2.A101 or SC2.A109) and washing three times in PBST for 10 minutes between blocking, primary antibody and secondary antibody incubations, respectively, Notum was detected with AP-AffiniPure Goat Anti-Mouse IgG, Fcγ Frag Specific (Jackson ImmunoResearch) at a dilution of 1:5000 in blocking buffer. Notum was then detected using NBT/BCIP substrate: a ready-to-use, precipitating substrate system for alkaline phosphatase. This substrate system produces an insoluble NBT diformazan end product that is blue to purple in color and can be observed visually.

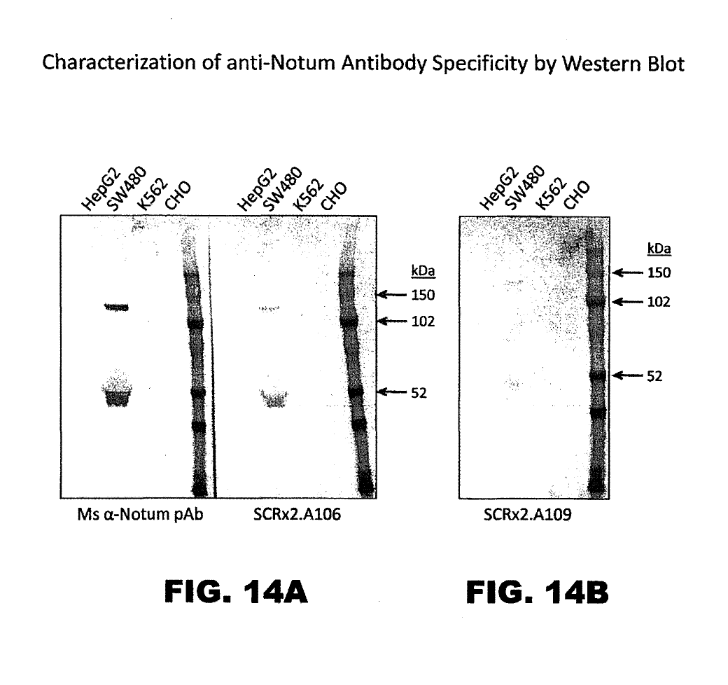

Each of the antibodies used to probe cell lysates detected human Notum in SW480 lysates, which appeared to be ˜50 kDa in size as a monomer and ˜125 kDa as a multimer (FIGS. 14A-14B). A slightly larger band in the range of ˜60 kDa, possibly representing an un-dimerized glycoform, was also observed when probed with all three antibodies.

### Example 21

### Differential Notum Protein Expression in Various Pooled Tissue Lysates

After documenting enhanced Notum gene expression in a number of tumorigenic samples as evidenced by the previous Examples, including reverse phase protein validation arrays comprising two pooled replicates of lysates from eleven different tumor types or their respective normal adjacent tissue (Example 6, OriGene Technologies) wherein Notum protein expression was detected using a mouse polyclonal antibody. Using the SCRx2.A109 mouse monoclonal antibody that recognizes human Notum by Western Blot (Example 20), more comprehensive reverse phase cancer protein lysate arrays comprising 4 dilutions of 432 tissue lysates from 11 tumor types, or their respective normal adjacent tissue, were obtained along with controls of 293 cells with or without TP53-overexpression as driven by an exogenous promoter (OriGene Technologies) were performed. Colorimetric detection reagents and protocols were provided by the manufacturer of the ProteoScan Arrays (OriGene Technologies), and spots on the fabricated array were converted to a digital image using a flatbed scanner using BZScan2 java Software (http://tagc.univ-mrs.fr/ComputationalBiology/bzscan/) to quantify Spot Intensity. Data was generated as described above and represented as average pixel intensity per spot. Data plotted represents individual spot densities for each tissue specimen, with a line representing the Geometric Mean.

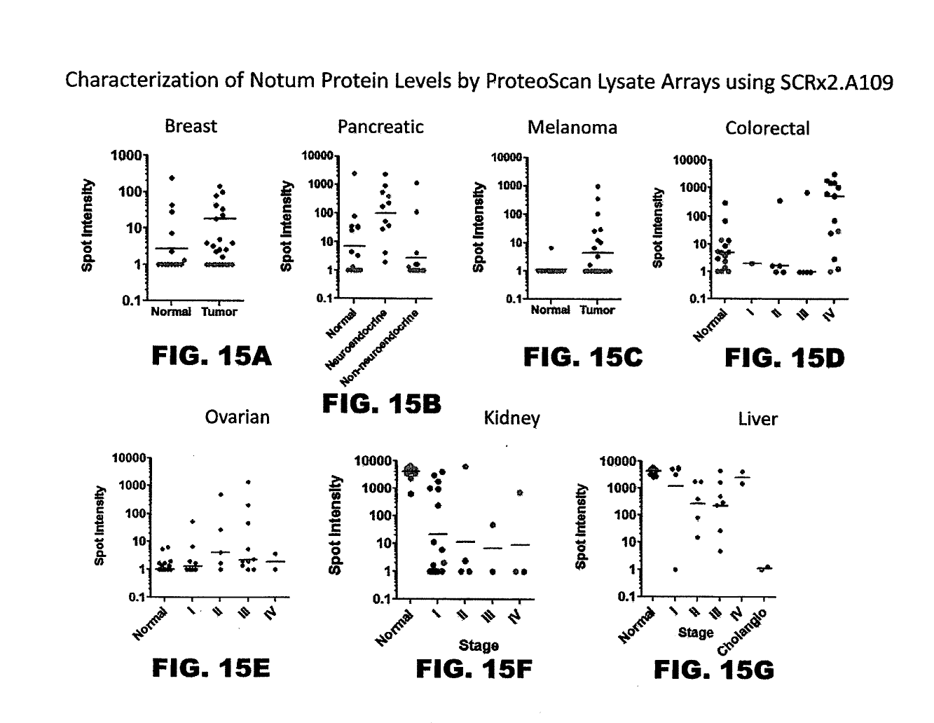

Results from these arrays are shown in FIGS. 15A-15G and indicate that expression of the Notum protein is upregulated in several different tumor types, including specific subpopulations of cancer patients. More specifically, FIGS. 15A-15G show that the levels of human Notum protein expression are elevated in subsets of patients with breast, colorectal and ovarian cancer, in addition to melanoma. Moreover, Notum protein expression appears elevated in most patients with the neuroendocrine-subtype of pancreatic cancer (FIG. 15B). Elevated Notum protein expression in various subsets of cancer patients, especially patients with late stage colorectal cancer and the pancreatic neuroendocrine subtype (islet cell tumors) of disease, suggest a role for Notum in promoting advanced disease and/or metastasis in these tumor types.

Also shown in the results in FIGS. 15F and 15G is the apparent reduction of Notum protein expression in kidney and liver tumors. This reduction is generally greater in later stages of disease, with the exception of stage IV liver cancer, and suggests that reduced local Notum levels may play a role in tumorigenesis and tumor progression. Though cholangiocarcinoma tumors have little Notum (FIG. 15G), cholangiocarcinoma is a cancer of the bile duct and no normal bile duct tissue was on the ProteoScan array for comparison.

### Example 22

### Notum Modulators Antagonize Notum Induced Cell Survival/Proliferation

As set forth in Examples 2 and 3, Notum expression was demonstrated to be elevated in tumor perpetuating cells from colorectal tumors. To determine whether Notum protein impacts cell proliferation and/or apoptosis of human colorectal cancer cells, HCT-116 cells or mouse lineage-depleted NTX tumor cells (i.e. human tumor cells) were plated as described below and exposed to recombinant hNotum (e.g. hNotum-His or hNotum-hFc) and anti-Notum antibodies. Cell numbers were then assessed 12-14 days later.

More specifically, mouse lineage-depleted NTX tumor cells from SCRx-CR4 or SCRx-CR42 tumors were plated at 20,000 cells/well in serum-free media that had previously been demonstrated to maintain tumorigenic cells in vitro followed 24-hours later by the addition of recombinant human Notum (His or hFc) in the presence or absence of Notum modulators SC2.D2.2 or SC2.10B3, or an isotype control antibody (i.e. MOPC). Cells were then incubated for 14 days at 37° C., 5% CO₂ and 5% O₂ and the number of viable cells was assessed using Promega's CellTiterGlo assay kit per the manufacturer's instructions. For the HCT-116 cell line (a commercially available colorectal tumor cell line), cells were plated at 2,000 cells per well in DMEM+1% FBS, followed 24-hours later by the addition of serum free DMEM containing recombinant human Notum in the presence or absence of monoclonal antibodies SC2.D2.2 or SC2.10B3. HCT-116 cells were then incubated for 12 days at 37° C., 5% CO2 and cell viability was assessed with Promega's CellTiterGlo assay kit. Higher readings are indicative of higher viable cell counts.

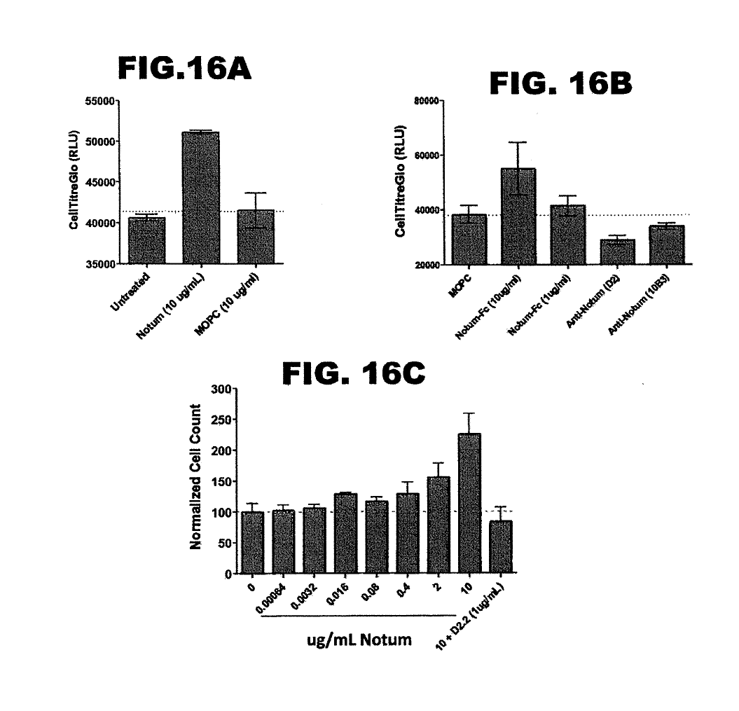

hNotum-His (10 μg/mL) exposure of mouse lineage-depleted NTX tumor cells from patient SCRx-CR42 (FIG. 16A) or hNotum-Fc (1 or 10 μg/mL) exposure of SCRx-CR4 (FIG. 16B) resulted in a 20-45% increase in cell counts compared to other untreated controls or cells exposed to the MOPC isotype control antibody. Conversely, exposure of SCRx-CR4 cells (expressing elevated levels of the Notum gene) to the human Notum neutralizing antibody SC2.D2.2 (10 μg/mL) showed significantly less proliferation compared to the appropriate MOPC isotype control antibody-treated cells (FIG. 16B). Similarly, the anti-Notum antibody SC2.10B3 (10 μg/mL) was also able to negatively impact cell numbers though not quite as effectively as SC2.D2.2 (FIG. 16B). Confirming the observations made immediately above, exposure of HCT-116 cells to 10 μg/mL of hNotum resulted in a more than 2-fold increase in cell numbers. Significantly, the increase in cell numbers as a result of hNotum exposure, which appeared to be dose dependent, was blocked by the presence of the anti-Notum monoclonal antibody SC2.D2.2 (FIG. 16C). These observations demonstrate that the human Notum protein (e.g. His or hFc forms) can increase cell proliferation and/or impair apoptosis, resulting in higher cell counts in the assays described above. Moreover, in accordance with the teachings herein the hNotum neutralizing monoclonal antibody SC2.D2.2 is able to block this activity and impairs Notum mediated proliferation.

### Example 23

### Notum Modulators Antagonize Notum Induced Esterase Activity

Aside from its orthologs found across animal species, human Notum is most closely related to plant pectin acetylesterases. It is also a member of the α/β hydrolase superfamily. These relationships suggest possible biochemical functions for the enzyme.

To test if Notum possesses carboxylesterase activity, purified recombinant hNotum-His was incubated with the chromogenic esterase substrates p-nitrophenyl acetate (PNPA) and p-nitrophenyl butyrate (PNPB) using standard assay conditions (West et al., PMID: 19225166). Briefly, PNPA or PNPB were dissolved/diluted in isopropanol to final concentrations of 10 mM. These substrate solutions were diluted 1:10 into assay buffer (0.1% gum arabic, 2.3 mg/mL sodium dexoycholate, 1×PBS) and incubated with defined amounts of hNotum enzyme, and the enzymatic release of the chromophore p-nitrophenol monitored by absorbance measurements at 405 nm.

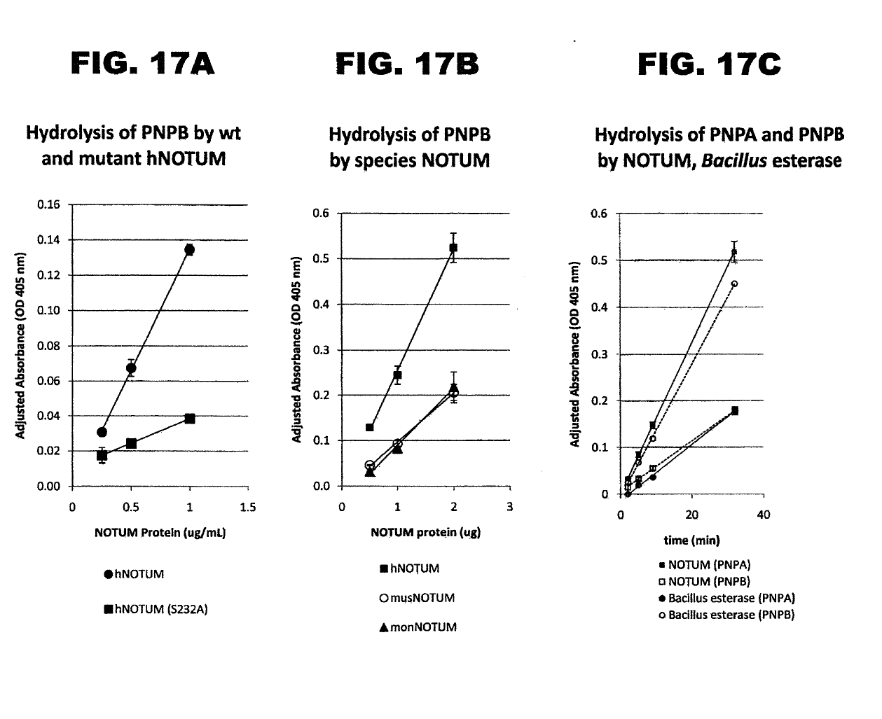

As can be seen in FIG. 17A, increasing amounts of hNotum release increasing amounts of p-nitrophenol from PNBA after 1 hour incubations at 37° C., demonstrating that Notum has carboxyesterase activity. Mutant Notum (S232A), in which the putative catalytic nucleophile has been altered by site-directed mutagenesis, showed a greatly reduced esterase activity. As shown in FIG. 17B, murine and macaque Notum proteins also display esterase activity. A recombinant esterase from Bacillus stearothermophilus (Sigma-Aldrich) was also included in the assay as a positive control (FIG. 17C). Specifically, FIG. 17C shows that at any specific time point hNotum yields a stronger signal for p-nitrophenol released from the PNPA (solid black squares and solid line) versus the PNPB substrates (open squares and dashed line), whereas the Bacillus esterase seems to preferentially hydrolyze the PNPB substrate (open circles and dashed line) versus the PNPA (solid circles and solid line). This data demonstrates that hNotum is able to induce esterase activity in a quantifiable manner.

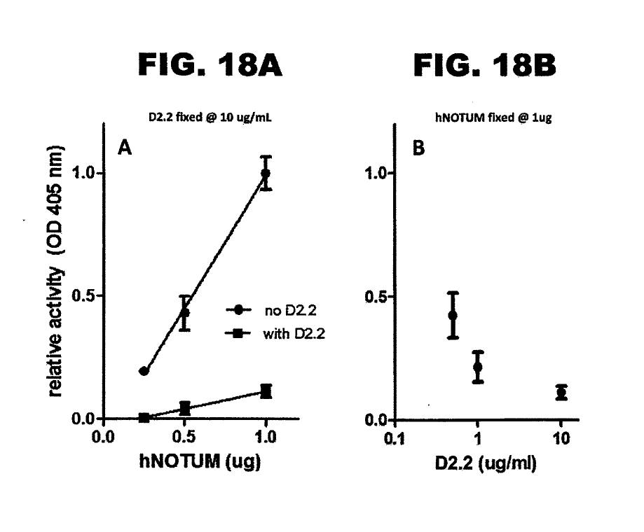

The results presented immediately above indicate that the measured esterase activity may be used to provide an assay that allows for the further characterization of the disclosed Notum modulators. In this respect, FIGS. 18A and 18B demonstrate that preincubation of hNotum protein with the Notum modulator SC2.D2.2 prior to addition of the PNPA and PNBA substrate results in greatly reduced esterase activity. This is entirely consistent with the data presented in previous examples and again demonstrates the ability of the SC2.D2.2 antibody to neutralize hNotum enzymatic function. More specifically, FIG. 18A shows a dose-response curve wherein the amount of SC2.D2.2 is fixed (none or 10 μg/mL) and Notum concentration is varied. As may be seen in FIG. 18A an increase in hNotum levels increases measured enzymatic activity even, to some extent, in the case where the SC2.D2.2 antibody is present. Conversely, FIG. 18B provides a dose response curve of measured enzymatic activity where the amount of hNotum is fixed at 1 μg/mL and the concentration of SC2.D2.2 is varied. The resulting curve clearly shows that the presence of a Notum modulator sharply reduces the amount of hNotum enzymatic activity in a concentration dependent manner. In contrast a control antibody (MOPC) has no effect on the esterase activity of Notum (data not shown).

Those skilled in the art will appreciate that the instant example demonstrates another assay that may be used to characterize the disclosed Notum modulators by measuring their impact on the enzymatic activity of Notum.

### Example 24

### Notum Modulators Antagonize Notum Induced Lipase Activity

Based on the characterization of Notum as a member of the α/β hydrolase superfamily and its demonstrated esterase activity, it was hypothesized that the protein may also act as a lipase. Those of skill in the art will appreciate that the lipase activity of proteins can be measured using a turbidometric assay measuring the lipolysis of Tween 20 (Pratt et al., 2000, PMID: 10706660). As such, experiments were conducted comprising the lipolysis of Tween 20 to measure the lipase activity of hNotum and provide yet another assay that could be used to characterize the Notum modulators of the instant invention.

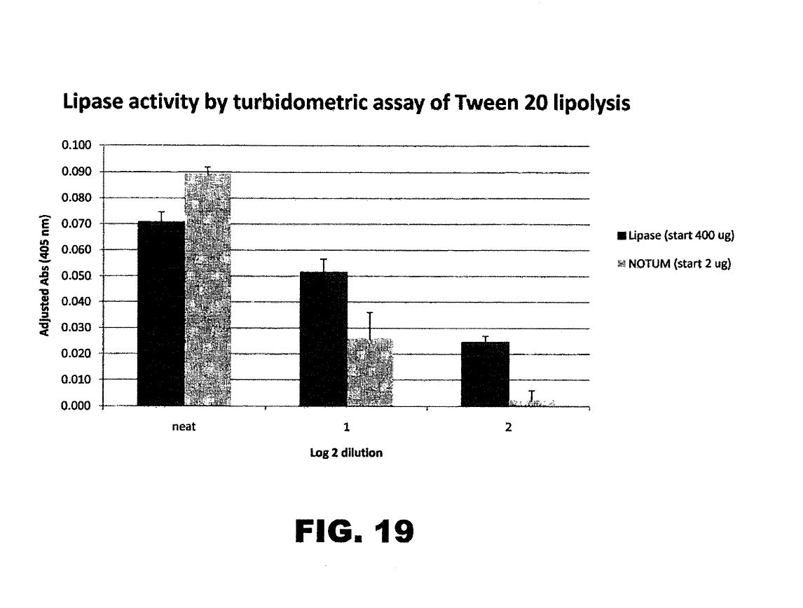

Briefly, recombinant hNotum (1 μg/well) was added to an assay buffer containing 50 mM Tris, pH 7.4, 33.3 mM CaCl₂, and 0.33% Tween-20. When the Tween 20 monolauryl group is cleaved by lipases (e.g. hNotum), the free fatty acid forms an insoluble complex with the Ca²⁺ cations resulting in a turbid solution, the OD of which can be measured at 405 nm to provide a measure of lipase activity. As a positive control, the activity of porcine pancreatic lipase (Sigma Aldrich) was measured in the same assay. FIG. 19 shows that purified recombinant Notum is capable of cleaving Tween 20 in a dose dependent fashion and demonstrates that such measurements provide yet another method by which to characterize the compounds of the instant invention.

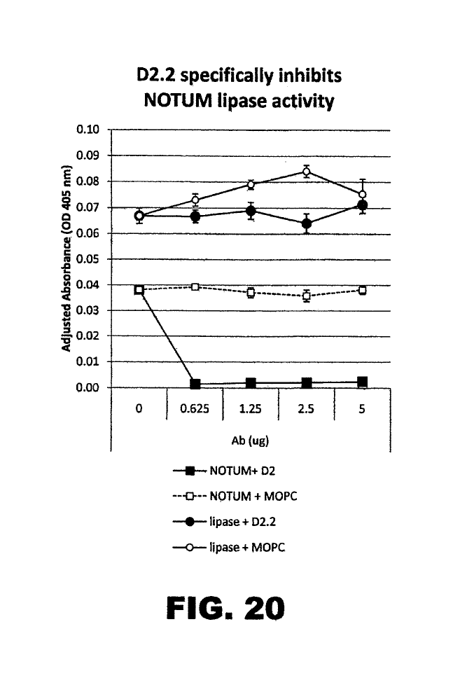

In order to take advantage of this enzymatic property and further exemplify the properties of the present invention, an assay was run to determine the effects of Notum modulators on the lipolytic activity of Notum. To that end, various concentrations of SC2.D2.2 were preincubated with hNotum for a set period prior to adding the mixture to the assay buffer and measuring the resulting enzymatic activity as described above. The results of the assay are graphically represented in FIG. 20

The resulting curves clearly show that almost all concentrations of the Notum modulator SC2.D2.2 substantially eliminate the lipase activity of Notum while not severely impacting the lipase activity of the porcine enzyme positive control. Further, FIG. 20 shows that the negative control antibody (MOPC) does not inhibit the lipase activity of either Notum or the porcine pancreatic lipase.

Such results clearly illustrate the ability of the disclosed Notum modulators to interfere or disrupt the enzymatic properties of the Notum protein and likely impact its inherent tumorigenic potential in a physiological setting.

### Example 25

### Fluorescent Assay of Notum Hydrolase Activity and Loss of Activity in Notum Modulators Comprising Point Mutations

In addition to the assays described in Examples 23 and 24, a fluorescent esterase substrate, 4-methylumbelliferyl heptanoate (Sigma), can be used to measure the activity of hydrolases using standard assay conditions (Richardson and Smith, 2007, PMID: 17620441; Jacks and Kircher, 1967, PMID: 5582971). Briefly, 4-MUH was dissolved in DMSO to a final concentration of 1.2 mM. This substrate was diluted 1:10 into assay buffer (0.1M Tris, pH 7.5, 50 mM NaCl, 0.05% Brij) and incubated with defined amounts of Notum enzyme or point-mutated Notum enzymes, and enzymatic release of the fluorescent molecule 4-methylumbelliferone monitored (355 nm excitation, 460 nm emission) using a Wallac Victor3 Multilabel Counter (Perkin Elmer).

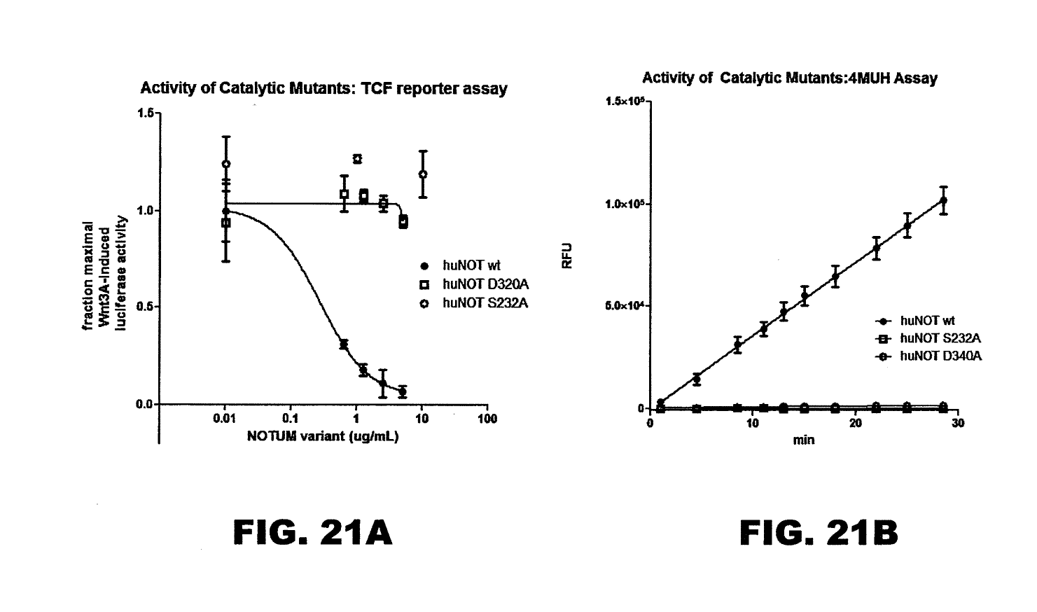

FIG. 21A shows that increasing amounts of wild-type human Notum enzyme can inhibit the response of the 293.TCF cells to Wnt3A in a dose-dependent fashion (assay details described in Example 14). However, the point mutants S232A and D340A show no ability to antagonize the activity of Wnt3A in the 293.TCF cells. Similarly, wild-type human Notum (62.5 ng per reaction) is capable of hydrolyzing the 4-MUH substrate, as demonstrated by a linear increase of relative fluorescence signal over time, whereas the S232A and D340A point mutants show no ability to hydrolyze the 4-MUH substrate (FIG. 21B).

### Example 26

### Notum Acts at a Step in the Canonical Wnt Pathway Upstream of Gsk3

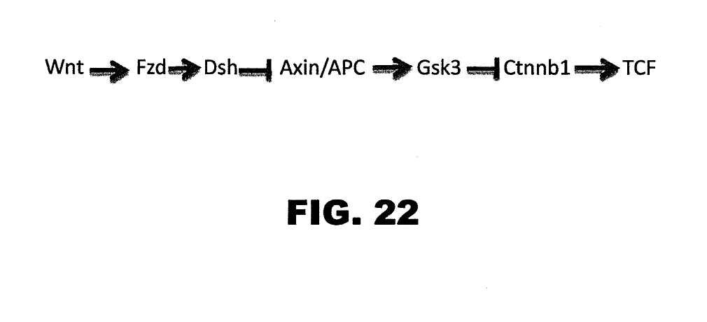

A simplified representation of the canonical (e.g. LEF/TCF) signaling pathway is represented in FIG. 22. Normally, beta catenin (CTNNB1) is rapidly turned over to the proteosome in the cytoplasm of cells following (1) its phosphorylation by GSK3 (and other kinases not depicted in the FIG. 22) when it is part of the AXIN/APC/GSK3 destruction complex in cells and (2) subsequent uniquitination. The binding of Wnt molecules to their receptor, Fzd, promotes phosphorylation of Dsh, which recruits Axin from the complex and causes the release of beta catenin from the destruction complex. This permits translocation of beta catenin to the nucleus of cells, where it complexes with LEF/TCF transcription factors to activate Wnt responsive genes. LiCl is a small molecule inhibitor of GSK3 (Klein and Melton, 1996, PMID: 8710892), which in the context of the canonical Wnt signaling pathway results in the downstream activation of Wnt responsive genes by promotion of beta catenin stabilization and release.

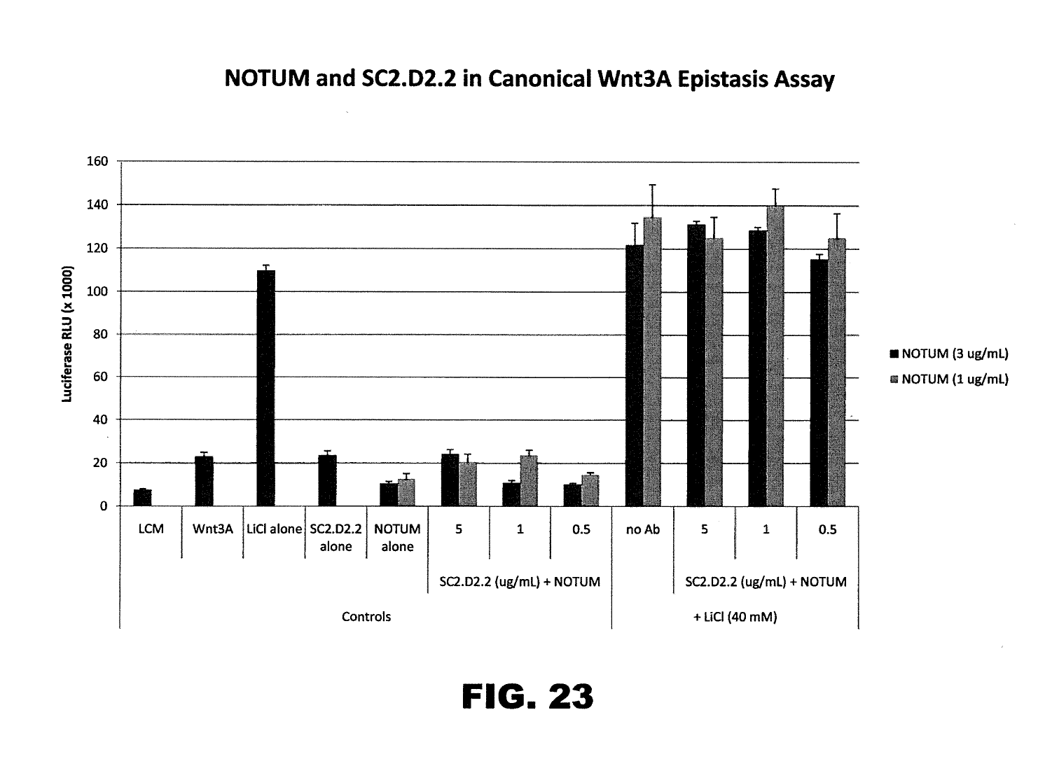

As can be seen in FIG. 23, Wnt3A CM and LiCl (40 mM) both activate luciferase transcription in the 293.TCF cells. Human Notum antagonizes Wnt3A CM, while SC2.D2.2 alone does not inhibit the induction of luciferase due to Wnt3A CM. However, SC2.D2.2 can inhibit the activity of human Notum in a dose dependent fashion, leading to restoration of Wnt3A-induced luciferase expression. Most importantly, LiCl is able to activate the luciferase reporter independent of the presence of human NOTUM and/or SC2.D2.2, indicating that Notum and the modulating antibody produce their effects upstream of GSK3.

### Example 27

### Delineation of Key Residues in the SC2.D2.2 Epitope Related to its Bioactivity

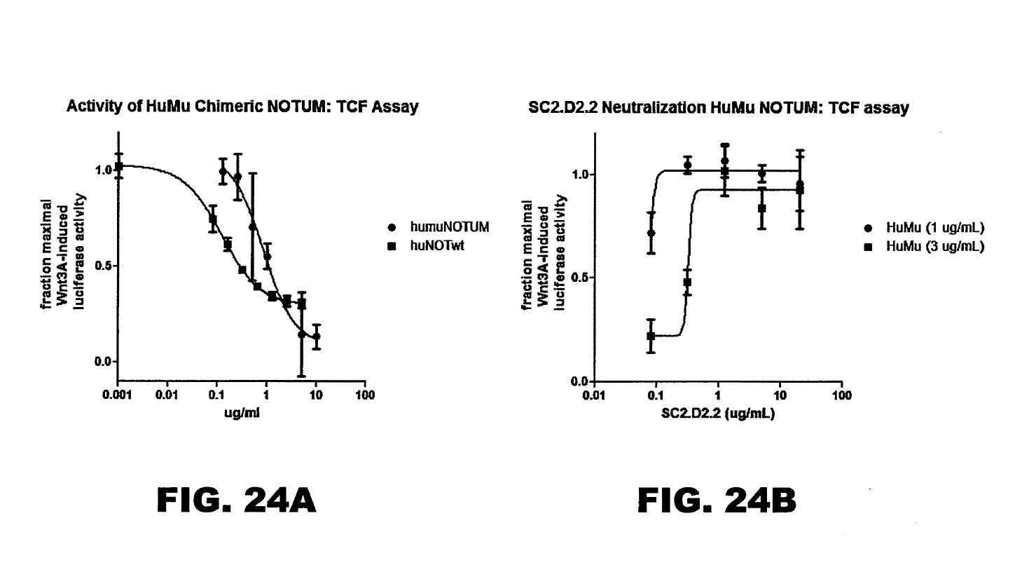

The chimeric human/mouse Notum protein described in Example 17 was placed into the 293.TCF assay. FIG. 24A shows that the chimeric molecule is able to inhibit induction of luciferase mediated by Wnt3A CM, although with lower efficacy than the wild-type protein. FIG. 24B shows this activity can be neutralized with SC2.D2.2, indicating that the epitope of SC2.D2.2 is contained with the first 144 residues of Notum, consistent with the ELISA data presented in Example 17. Taken together, the ability of SC2.D2.2 to neutralize the bioactivity of the chimeric molecule, the activity data of SC2.D2.2 against various species forms of Notum as shown in Example 18 and the sequence alignment as set forth FIG. 1C, suggest that the D141 residue of human Notum might be a critical residue in the epitope of SC2.D2.2. (Note that FIG. 1C would annotate the residue as D129, based upon numbering from the start of the mature Notum protein, whereas the D141 annotation is based upon the consideration of the wild-type human protein precursor).

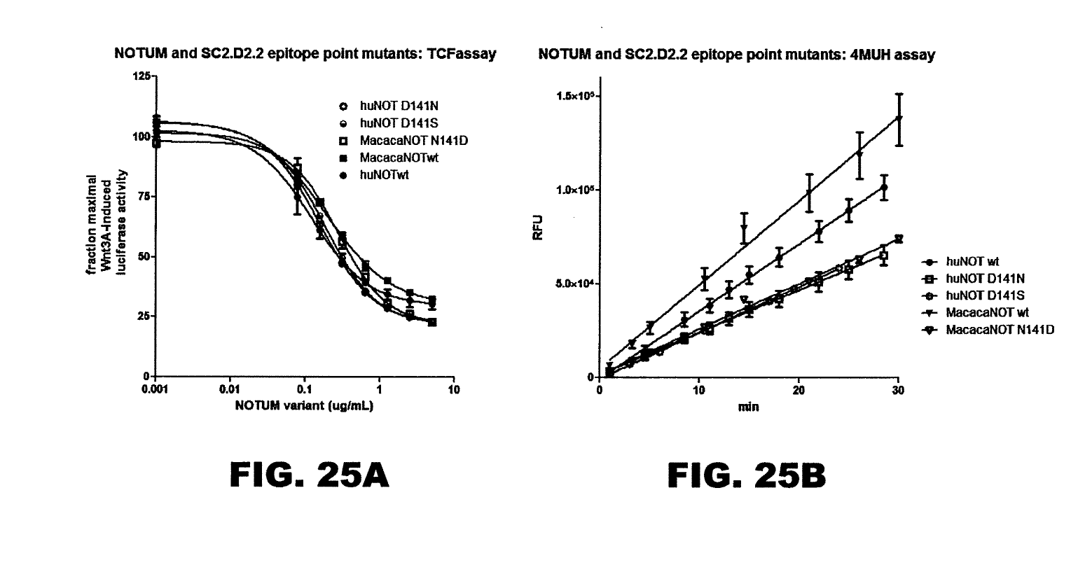

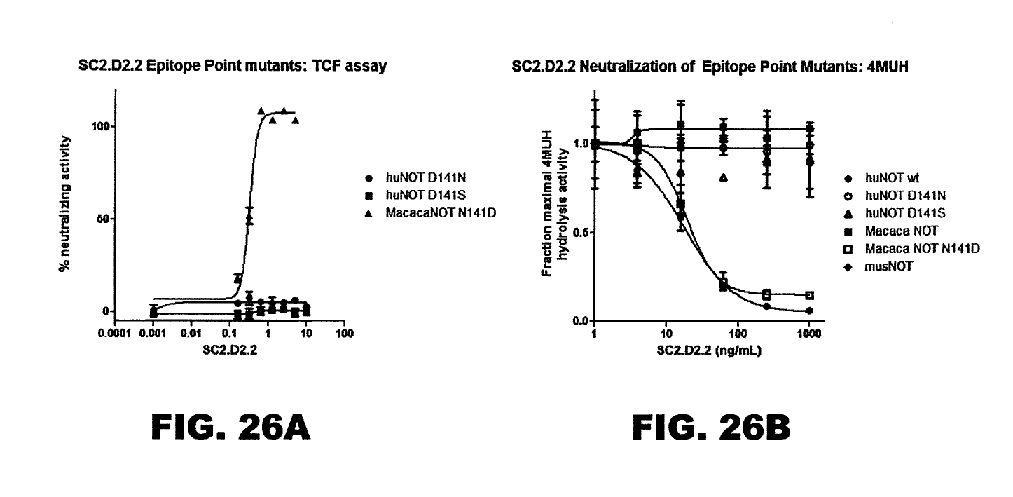

To formally demonstrate the importance of the residue in the epitope, standard molecular biology techniques were employed to point mutate this residue in human Notum, to either the macaque (D141N) or the murine (D141S) residue. Similarly, the macaque residue at this position was point mutated to the human residue (N141D). FIG. 25 shows that each of these point mutations yielded a protein that retained bioactivity in the 293.TCF assay (FIG. 25A) and the 4-MUH hydrolysis assay (FIG. 25B). However, these point mutants differed in their ability to be neutralized by SC2.D2.2 (FIG. 26). Mutation of the human residue to either macaque or murine residues eliminated the ability of SC2.D2.2 to neutralize the mutant Notum protein (FIG. 26A), whereas changing the macaque protein residue to the human residue (N141D) now enabled SC2.D2.2 to neutralize the mutant protein (FIG. 26A) despite being unable to neutralize the wild-type macaque protein (FIG. 12A). This pattern of neutralizing behavior by SC2.D2.2 was also observed for the mutant proteins in the 4-MUH assay (FIG. 26B): changing of the human D141 residue eliminated the ability of SC2.D2.2 to neutralize the resultant protein (D141N, D141S), whereas changing of the macaque residue N141 enabled the antibody to neutralize the mutant protein (macaque N141D). These data clearly demonstrate the importance of the D141 residue for the ability of SC2.D2.2 to neutralize the bioactivity of the Notum protein.

### Example 28

### Incubation of Notum with rhWnt3A Leads to Inactivation of Wnt Activity

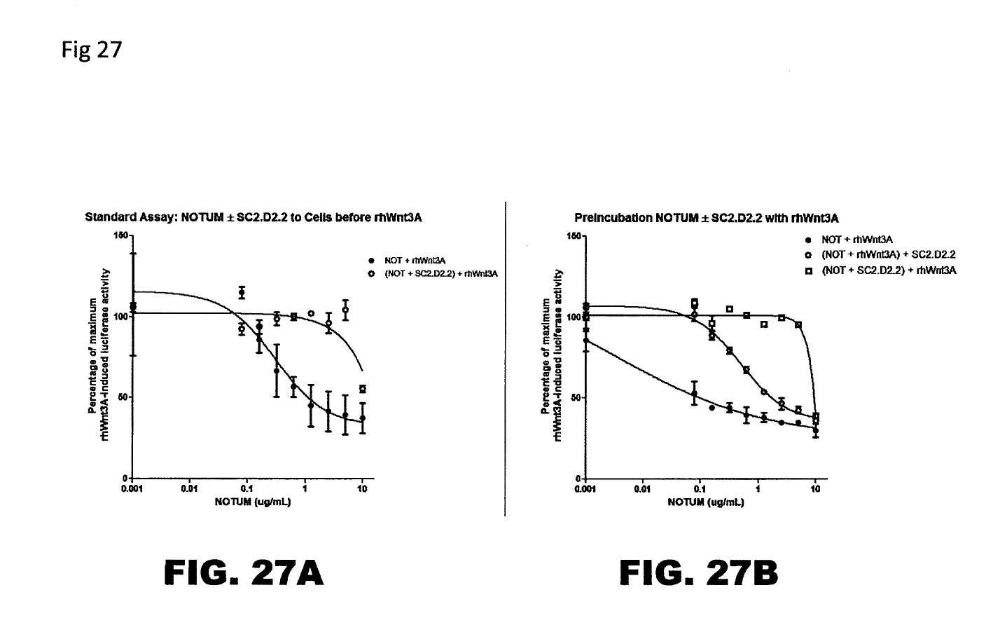

In order to determine the kinetics of Notum mediated antagonism of Wnt3A signaling, recombinant Notum alone or in the presence of SC2.D2.2 was preincubated with recombinant human Wnt3A (rhWnt3A) for 2 hours at 37° C., prior to addition of the complexes to 293.TCF cells. This resultant induction of luciferase by the rhWnt3A was compared to that observed using the standard protocol for the 293.TCF assay, in which Notum alone or Notum+SC2.D2.2 was added to the cells for two hours prior to the addition of rhWnt3A. As can be seen in FIG. 27A, the standard assay conditions show that Notum in the absence of SC2.D2.2 is capable of inhibiting induction of luciferase in the 293.TCF cells exposed to 250 ng/mL rhWnt3A (closed circles), and that incubation of Notum with 10 μg/mL SC2.D2.2 prior to addition of rhWnt3A blocks the ability of Notum to antagonize the rhWnt3A (open circles). Of interest however, is the preincubation of Notum and rhWnt3A prior to addition to the 293.TCF cells. In this case the response of the cells to rhWnt3A is reduced greatly (closed circles FIG. 27B). In contrast, complexing of Notum with SC2.D2.2 prior to preincubation with rhWnt3A restores the sensitivity of the cells to rhWnt3A (open squares FIG. 27B). Together, these data suggest that Notum may be directly inactivating rhWnt3A, as opposed to interacting with a molecule on the presence of the cell surface.

### Example 29

### Small Molecule Inhibition of Notum Activity

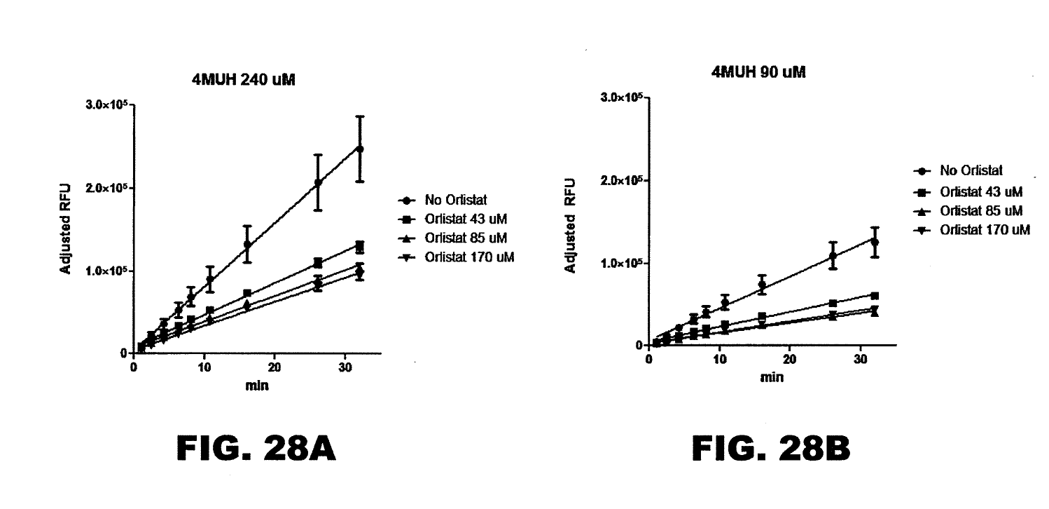

The studies shown in Examples 23, 24, and 25 indicate that Notum possesses the ability to hydrolyze esters and lipids, while the data presented in Example 28 suggests that may act directly on rhWnt3A. Consistent with a putative hydrolase activity for Notum, it could be hypothesized that this inactivation is related to the ability of Notum to delipidate Wnt3A. Two lipids are known to be linked to Wnt3A, a saturated palmitate chain at Cys77 and an unsaturated palmitoleoylic chain at S209 (Lorenowicz and Korswagen, 2009, PMID: 19559695). Both lipid chains have been suggested to be important for secretion of Wnt3A as well as signaling (Franch-Marro et al, 2008, PMID: 18430784). Because palmitate is linked to Wnt3A via a thioester linkage at Cys77, this would suggest that Notum might be inactivated by a known inhibitor of a thioesterase enzyme. One such small molecule is orlistat (Xenical®), which has been shown to inhibit the thioesterase subunit of the multisubunit enzyme fatty acid synthase (Kridel et al, 2004, PMID: 15026345). Therefore, the 4-MUH assay described in example 25 was performed in the presence of varying amounts of 4-MUH substrate (240 μM or 90 μM) and orlistat (0-170 μM). As can be seen in FIG. 28, orlistat inhibits the hydrolysis activity of Notum upon 4MUH in a dose-dependent fashion, demonstrating the ability of both small molecules and a known lipase-inhibiting drug to inhibit Notum.

### Example 30

### Changes in the Physical Behavior of Wnt3A in Response to Incubation with Notum

If Notum directly acts upon Wnt3A to delipidate the protein, this cleavage should result in a change in the hydrophobicity of the protein, which can be measured by a change in its partitioning behavior between aqueous and detergent phases in a Triton X114 partition assay (Bordier, 1981, PMID: 6257680): lipidated Wnt3A will be found in the aqueous phase, whereas delipidated Wnt3A should show up in the aqueous phase (Willert et al., 2003, PMID: 12717451). To demonstrate the enzymatic properties of Notum, 1.5 μg of rhWnt3A in 0.1% BSA (R & D Systems) was incubated overnight with 250 ng of Notum at room temperature. An equal volume of 4.5% Triton X114 was added to the mixture, the mixture incubated on ice for 5 minutes, then at 37° C. for five minutes, before separating the phases using centrifugation at 2000×g for five minutes at room temperature. Following separation each sample was adjusted to normalize the ionic strength and Triton X114 content before analyzing the aliquots by PAGE electrophoresis. After running the gel the protein bands were transferred to a membrane for immunoblotting using an anti-rhWnt3A antibody (Cell Signaling Technology). Bands were visualized using SuperSignal West Pico Chemiluminescent substrate (Thermo Fisher Scientific).

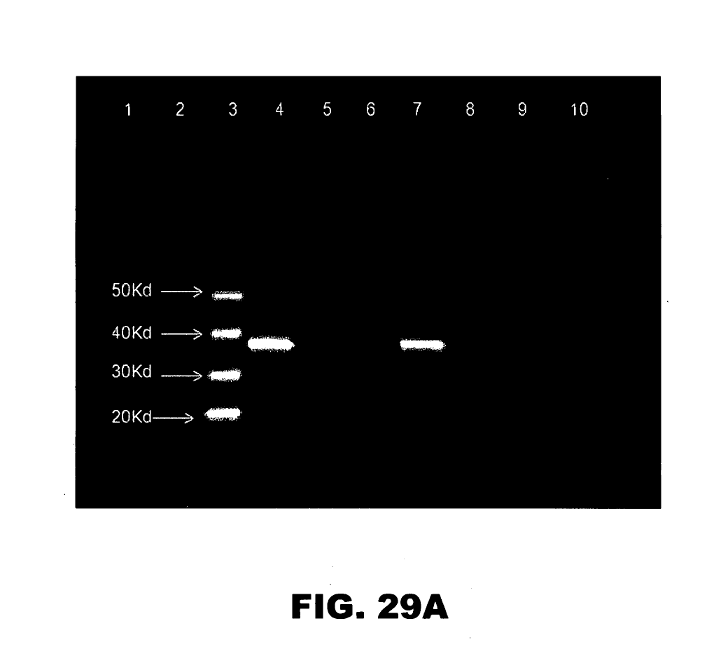

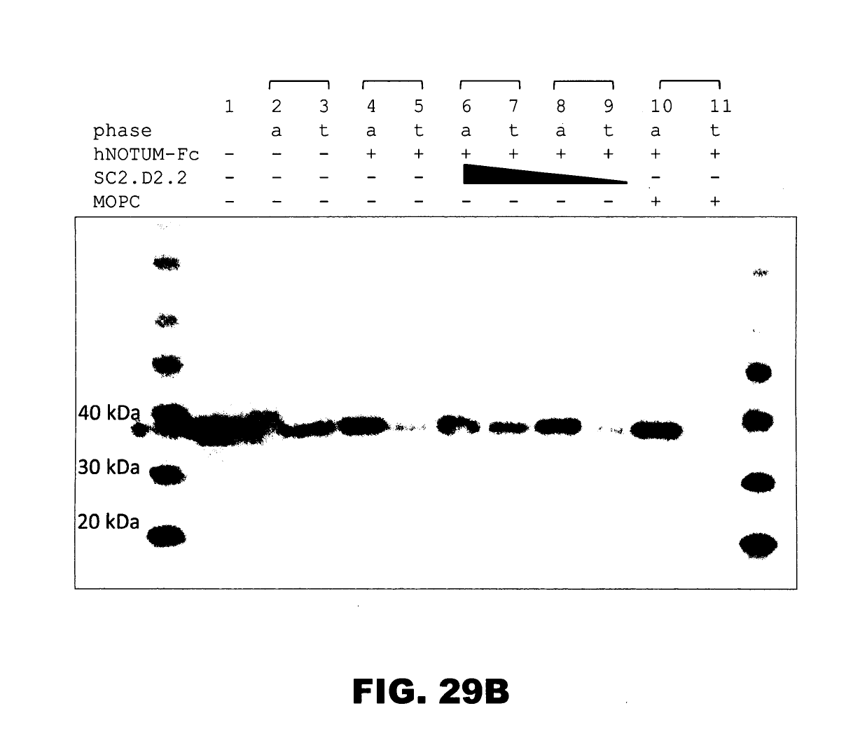

As can be seen in the blot shown in FIG. 29A, in the absence of Notum rhWnt3A appears only in the Triton X114 phase (lane 6) and not in the aqueous phase (lane 5). Conversely, incubation of rhWnt 3A with Notum leads to appearance of rhWnt3A in the aqueous phase (lane 8) as well as the Triton X114 lane (lane 9). These data are suggestive of the ability of Notum to delipidate Wnt3A. However it is possible that such delipidation is incomplete under the instant experimental conditions thereby leading to the observed retention of some rhWnt3A in the Triton X114 phase.

It is also interesting that Notum has been linked to modulation of the Sonic Hedgehog (Shh) in Drosophila (Ayers et al, 2010, PMID: 20412775). Shh is another lipid modified protein, specifically one containing a palmitic acid chain esterified through the alpha-amino group of the mature protein N-terminal Cys24 (Pepinsky et al, 2008, PMID: 9593755). Thus, the previously described genetic interactions of Notum with the Hedgehog signaling pathway may also reflect a lipase-based delipidation of Hedgehog proteins, disregulating their signaling properties, with consequential effects in the promotion of oncogenesis.

In any event the demonstrated ability of Notum to change the physiochemical behavior of rhWnt3A can be blocked by the Notum modulator SC2.D2.2 as shown in FIG. 29B. Lane 1 is a positive molecular weight marker for rhWnt3A while the presence or absence of reagents in each aliquot is noted above the respective lane (where a is the aqueous fraction and t is Triton X-114 fraction, and the sliding bar indicates the concentration of Notum modulator). Again, untreated rhWnt3A appears only in the Triton X114 phase (lane 3) but not the aqueous phase (lane 2). Overnight incubation with hNotum-Fc again leads to a redistribution of the rhWnt3A into the aqueous phase (compare lanes 4 and 5). This redistribution can be blocked if hNotum-Fc is first preincubated with SC2.D2.2 (compare lanes 6 and 7 versus lanes 4 and 5, respectively). The blocking effect is dependent upon the amount of SC2.D2.2 used; higher amounts of SC2.D2.2 result in more of the rhWnt3A being retained in the Triton X114 phase (compare lanes 7 and 9). The blocking of redistribution is also dependent upon the specificity of the Notum modulator; no blocking of redistribution is observed if hNotum-Fc is first preincubated with a control monoclonal antibody, MOPC (lanes 10 and 11).

### Example 31

### Modulation of Human, Murine and Monkey Notum

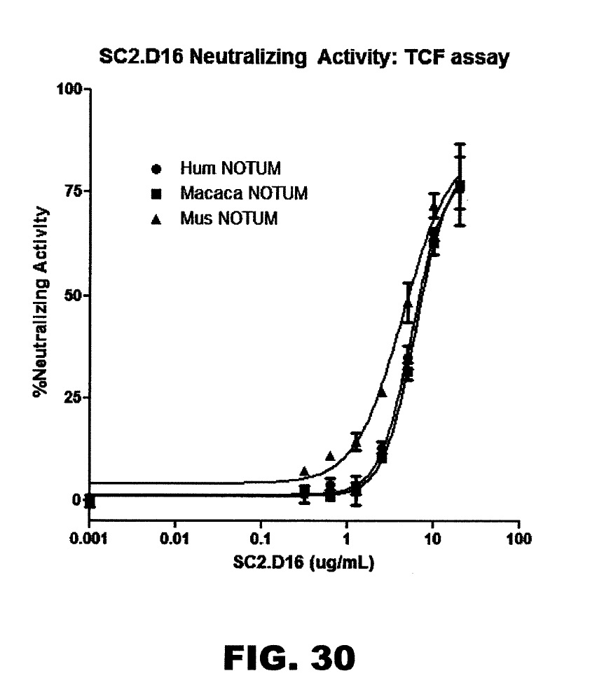

As demonstrated above the monoclonal antibody SC2.D2.2 has been shown to specifically inhibit the human version of Notum without inhibiting murine or macaque versions of the protein. A second monoclonal antibody modulator of human Notum, SC2.D16, was characterized for its ability to inhibit mouse and macaque Notum using the 293.TCF assay described in Example 14 above. As shown in FIG. 30, SC2.D16 inhibits human and monkey Notum with similar efficacy, and may be slightly more potent against murine Notum than either of the primate Notum proteins.

### Example 32

### Humanization of a Monoclonal Antibody Notum Modulator

Murine antibody SC2.D2.2 was humanized using a computer-aided CDR-grafting method (Abysis Database, UCL Business Plc.) and standard molecular engineering techniques to provide hSC2.D2.2 modulator. The human framework regions of the variable regions were selected based on their highest sequence homology to the mouse framework sequence and its canonical structure. For the purposes of the analysis the assignment of amino acids to each of the CDR domains is in accordance with the Chothia et al. numbering. Several humanized antibody variants were made in order to generate the optimal humanized antibody. A chimeric version of the murine antibody comprising the entire murine light and heavy variable regions and a human constant region was also fabricated for purposes of evaluation.

Molecular engineering procedures were conducted using art-recognized techniques. To that end total mRNA was extracted from SC2.D2.2 hybridoma according to the manufacturer's protocol (Trizol® Plus RNA Purification System, Life Technologies). A primer mix comprising thirty-two mouse specific 5′ leader sequence primers, designed to target the complete mouse repertoire, was used in combination with 3′ mouse Cγ1 primer to amplify and sequence the variable region of SC2.D2.2 heavy chain. Similarly thirty-two 5′ Vk leader sequence primer mix designed to amplify each of the Vk mouse families combined with a single reverse primer specific to the mouse kappa constant region were used to amplify and sequence the kappa light chain. The V_{H} and V_{L} transcripts were amplified from 100 ng total RNA using reverse transcriptase polymerase chain reaction (RT-PCR).

A total of eight RT-PCR reactions were run for the SC2.D2.2 hybridoma: four for the V kappa light chain and four for the V gamma heavy chain (γ1). The QIAGEN One Step RT-PCR kit was used for amplification, (Qiagen, Inc.). The extracted PCR products were directly sequenced using specific V region primers. Nucleotide sequences were analyzed using IMGT to identify germline V, D and J gene members with the highest sequence homology. The derived sequences were compared to known germline DNA sequences of the Ig V- and J-regions using the V-BASE2 and by alignment of V_{H} and V_{L} genes to the mouse germ line database.

Sequence analysis: from the nucleotide sequence information, data regarding V, D and J gene segment of the heavy and light chain of SC2.D2.2 were obtained. Based on the sequence data new primer sets specific to the leader sequence of the Ig V_{H} and V_{K} chain of SC2.D2.2 were designed for cloning of the recombinant mouse D2 monoclonal antibody. Subsequently the V-(D)-J sequences were aligned with mouse Ig germ line sequences. Heavy chain genes of SC2.D2.2 were identified as IGHV5-17, DQ52a.1 and JH1. Light chain genes were from V kappa IGKV3-12 and Jkappa5, germline gene families.

The obtained heavy and light chain sequences were aligned to the functional human variable region sequences. Sequence homology was found to be 81% and 62% identity to the germ line sequence of Human V_{H}3-48 and V_{K} A19 respectively. These germ lines were picked as the human framework for the humanized SC2.D2.2 mAb. Nucleotide sequences were designed to encode the protein sequences of the humanized V_{L} and V_{H}, generally using codons found in the human and mouse sequence. Synthetic DNA fragments of each V gene were synthesized by Integrated DNA Technologies, Inc.

In FIGS. 31A and B sequences of the humanized SC2.D2.2 heavy (FIG. 31A) and light (FIG. 31B) chain V domains (upper sequences—SEQ ID NOs: 331 and 332) aligned with respective murine SC2.D2.2 V domains (lower sequences—SEQ ID NOs: 56 and 58). Vertical marks indicate that the amino acids in the murine and humanized versions are identical. CDRs as defined by Chothia et al. are underlined. Once the variable regions were generated, humanized and chimeric antibodies were produced for further characterization.

For antibody production directional cloning of the murine and humanized variable gene PCR products into human immunoglobulin expression vectors was undertaken. All primers used in Ig gene-specific PCRs included restriction sites (AgeI and XhoI for IgH, XmaI and DraIII for IgK, which allowed direct cloning into expression vectors containing the human IgG1, and IGK constant regions, respectively. In brief, PCR products were purified with Qiaquick PCR purification kit (Qiagen, Inc.) followed by digestion with AgeI and XhoI (IgH), XmaI and DraIII (IgK), respectively. Digested PCR products were purified prior to ligation into expression vectors. Ligation reactions were performed in a total volume of 10 μL with 200U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto ampicillin plates (100 μg/mL). The AgeI-EcoRI fragment of the V_{H} region was than inserted into the same sites of pEE6.4HuIgG1 expression vector while the synthetic XmaI-DraIII V_{K} insert was cloned into the XmaI-DraIII sites of-the respective pEE12.4Hu-Kappa expression vector.

Cells producing humanized (i.e. hSC2.D2.2) antibody and chimeric SC2.D2.2 antibody were generated by transfection of HEK 293 cells with the appropriate plasmids using 293fectin. In this respect plasmid DNA was purified with QIAprep Spin columns (Qiagen). Human embryonic kidney (HEK) 293T (ATCC No CRL-11268) cells were cultured in 150 mm plates (Falcon, Becton Dickinson) under standard conditions in Dulbecco's Modified Eagle's Medium (DMEM) supplemented with 10% heat inactivated FCS, 100 μg/mL streptomycin, 100 U/mL penicillin G (all from Life Technologies).

For transient transfections cells were grown to 80% confluency. Equal amounts of IgH and corresponding IgL chain vector DNA (12.5 μg of each vector DNA) was added to 1.5 mL Opti-MEM mixed with 50 μL HEK 293 transfection reagent in 1.5 mL opti-MEM. The mix was incubated for 30 min at room temperature and distributed evenly to the culture plate. Supernatants were harvested three days after transfection, replaced by 20 mL of fresh DMEM supplemented with 10% FBS and harvested again at day 6 after transfection. Culture supernatants were cleared from cell debris by centrifugation at 800×g for 10 min and stored at 4° C. Recombinant chimeric and humanized antibodies were purified with Protein G beads (GE Healthcare).

### Example 33

### Characterization of Monoclonal Antibody Notum Modulators

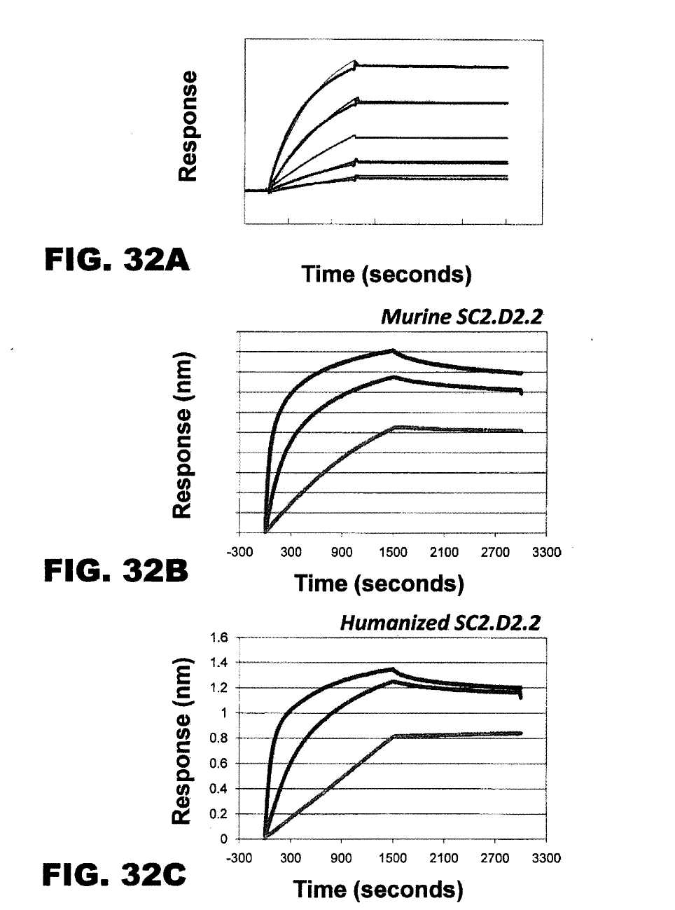

Three methods were used to characterize the affinity of humanized SC2.D2.2 relative to its analogous mAb with the murine variable region. First, binding signal was measured for a fixed amount of antibody probed against serial dilutions of antigen in an ELISA format. Measured signal levels were substantially similar (data not shown). Second, the affinity of murine SC2.D2.2 was measured by Biacore using surface plasmon resonance (SPR) to provide the results set forth in FIG. 32A. Based on a concentration series of 12.5, 6.25, 3.125, 1.5625, 0.78125 nM and using a 1:1 Langmuir binding model, the K_{d} of the antibody binding to antigen was estimated to be less than 0.1 nM. Long off-rates for this interaction made accurate determination of affinity through kinetics difficult. The murine antibody was then directly compared to the humanized derivative using bio-layer interferometry analysis on a ForteBIO RED (ForteBIO, Inc.) with a concentration series of 250, 125, and 62.5 nM antigen. As seen in FIG. 32B (murine variable region) and FIG. 32C (humanized variable region) each of the antibodies showed excellent affinity and produced nearly identical binding curves. It will be appreciated that the similarity of the curves indicates that the humanization process did not adversely impact the kinetics of the derivatized antibody.

### Example 34

### Notum Modulators May be Used as Diagnostic Agents

In accordance with the teachings herein, the disclosed Notum modulators may be used as diagnostic agents to detect Notum associated biomarkers in biological samples from patients.

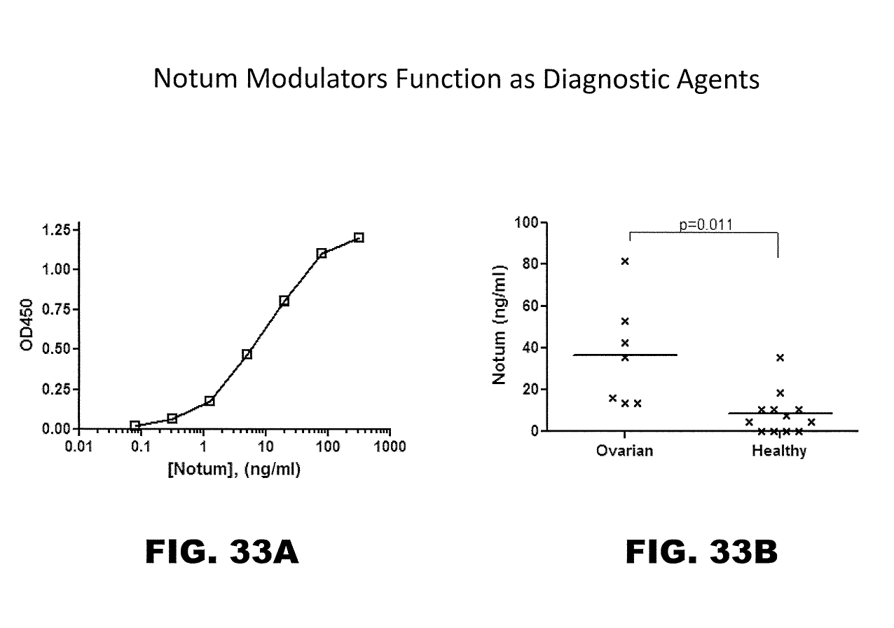

Notum is known to be secreted to some extent and may act in a paracrine fashion on neighboring cells either as soluble molecule in extracellular fluids or by association with extracellular matrix. Exhibiting such properties Notum should be detectable in body fluids such as serum or plasma in certain disease conditions and could therefore be useful for diagnostic purposes or serve as disease biomarker. To confirm this aspect of the invention a standard curve was generated with anti-Notum antibodies using a sandwich ELISA format as shown in FIG. 33A. The resulting curve was then used to quantitate Notum levels in plasma samples obtained from healthy subjects and patients suffering from ovarian cancer as shown in FIG. 33B.

More specifically, murine SC2.D2.2 was absorbed on standard ELISA plates at 2 μg/ml in a 50 mM sodium carbonate buffer at pH9.6. After washing the plates with PBS containing 0.05% (v/v) Tween-20 (PBST), the plates were blocked in PBS containing 2% (w/v) bovine serum albumin (BSA buffer) for two hours at ambient temperature. The content of the plates was flicked off, and purified recombinant Notum-His at varying concentrations (i.e., to provide the standard curve) or patient samples diluted in BSA buffer were added to the plates for a minimum of two hours at ambient temperature. The plates were washed in PBST before adding Notum-specific mouse polyclonal antibody conjugated to biotin at 0.5 μg/ml in BSA buffer. After incubation for one hour, the plate was washed again with PBST and incubated for 30 minutes with a 1:2000 dilution of Streptavidin conjugated to horse radish peroxidase (Jackson Immuno Research). After washing all plates twice with PBST, 100 μl TMB substrate (Thermo Scientific) was added to the wells and incubated for 30 minutes in the dark. Color reaction was stopped by adding 100 μl/well 2M sulfuric acid. Absorbance at OD 450 nm was read in all wells using a standard plate reader.

Using values extrapolated from the standard curve in FIG. 33A, the ELISA sandwich format permits sensitive detection of Notum analyte concentration in patient plasma samples. More particularly, FIG. 33B shows the derived Notum analyte concentrations in plasma samples from healthy adults (n=12) and a group of ovarian cancer patients (n=7) in disease stages 2-4. The data show that average Notum concentrations in plasma samples of healthy adults is approximately 8.6±10.3 ng/ml while Notum concentration in ovarian cancer patients appears significantly higher at 36.5±25.2 ng/ml. These results clearly demonstrate that the disclosed modulators of the instant invention can effectively act as a diagnostic agent for the detection and/or monitoring of neoplastic disorders.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1-101. (canceled)

102. An isolated anti-Notum antibody which competes with hSC2.D2.2 antibody for binding to Notum.

103. The anti-Notum antibody of claim 102, comprising a heavy chain and a light chain; wherein
the heavy chain comprises CDR1 comprising the amino acid sequence of SEQ ID NO:122, CDR2 comprising the amino acid sequence of SEQ ID NO:160, and CDR3 comprising the amino acid sequence of SEQ ID NO:198; and
the light chain comprises CDR1 comprising the amino acid sequence of SEQ ID NO:236, CDR2 comprising the amino acid sequence of SEQ ID NO:274, and CDR3 comprising the amino acid sequence of SEQ ID NO:312.

104. The anti-Notum antibody of claim 103, wherein the heavy chain comprises a variable region comprising the amino acid sequence of SEQ ID NO:331 and the light chain comprises a variable region comprising the amino acid sequence of SEQ ID NO:332.

105. The anti-Notum antibody of claim 103, wherein the antibody is hSC2.D2.2 antibody.

106. An isolated anti-Notum antibody comprising CDR1, CDR2, and CDR3 of the heavy chain and CDR1, CDR2, and CDR3 of the light chain of an antibody selected from SC2.A1 antibody, SC2.A3 antibody, SC2.A6 antibody, SC2.A8 antibody, SC2.A11 antibody. SC2.A12 antibody, SC2.A13 antibody, SC2.A101 antibody, SC2.A109 antibody, SC2.A113 antibody, SC2.A106 antibody, SC2.A122 antibody, SC2.10E11 antibody, SC2.9E7 antibody, SC2.A118 antibody, SC2.A126 antibody, SC2.5C4 antibody, SC2.A110 antibody, SC2.D1 antibody, SC2.D3 antibody, SC2.D4 antibody, SC2.D7 antibody, SC2.D12 antibody, SC2.D14 antibody, SC2.D15 antibody, SC2.D16 antibody, SC2.D19 antibody, SC2.D22 antibody, SC2.D23 antibody, SC2.D27 antibody, SC2.D28 antibody, SC2.D30 antibody, SC2.D41 antibody, SC2.D45 antibody, SC2.D46 antibody, SC2.D47 antibody, and SC2.D57 antibody.

107. The antibody of any one of claims 102 to 106, wherein the antibody is conjugated to an anti-cancer agent.

108. The antibody of any one of claims 102 to 107, wherein the antibody is a chimeric antibody or a humanized antibody.

109. A method of treating a patient having at least one neoplastic disorder selected from adrenal cancer, bladder cancer, cervical cancer, endometrial cancer, kidney cancer, liver cancer, lung cancer, ovarian cancer, colorectal cancer, pancreatic cancer, prostate cancer, breast cancer, and cancer metastasis, comprising administering a therapeutically effective amount of an antibody of any one of claims 102 to 108 to the patient.

110. The method of claim 109 wherein the patient has a solid tumor exhibiting at least one mutation selected from a KRAS mutation, an APC mutation, and a CTNNB1 mutation.

111. The method of claim 109 or claim 110, wherein the treatment reduces the frequency of tumor initiating cells in the patient, wherein the reduction in frequency is determined: using flow cytometric analysis of tumor cell surface markers known to enrich for tumor initiating cells; using immunohistochemical detection of tumor cell surface markers known to enrich for tumor initiating cells; using in vitro or in vivo limiting dilution analysis; using in vivo limiting dilution analysis comprising transplant of live human tumor cells into immunocompromised mice; using in vivo limiting dilution analysis comprising quantification of tumor initiating cell frequency using Poisson distribution statistics; using in vitro limiting dilution analysis comprising limiting dilution deposition of live human tumor cells into in vitro colony supporting conditions; or using in vitro limiting dilution analysis comprises quantification of tumor initiating cell frequency using Poisson distribution statistics.

112. A composition comprising an antibody of any one of claims 102 to 108 and a pharmaceutically acceptable carrier.

113. An isolated composition comprising a first polynucleotide and a second polynucleotide, wherein the first polynucleotide encodes a heavy chain and the second polynucleotide encodes a light chain, and wherein the first and second polynucleotides encode an antibody of any one of claims 102 to 108.

114. An isolated host cell comprising a first polynucleotide and a second polynucleotide, wherein the first polynucleotide encodes a heavy chain and the second polynucleotide encodes a light chain, and wherein the first and second polynucleotides encode an antibody of any one of claims 102 to 108.

115. A method of diagnosing a hyperproliferative disorder in a patient comprising:
a. obtaining a tissue sample from the patient;
b. contacting the tissue sample with an anti-Notum antibody of any one of claims 102 to 108; and
c. detecting or quantifying the anti-Notum antibody associated with the sample.


SEQUENCE LISTING (334 sequences)

SEQ ID NO: 1 - DNA, 1491 bp, Homo sapiens
atgggccgaggggtgcgcgtgctgctgctgctgagcctgctgcactgcgccgggggcagcgagggcaggaagacctggcggcgccggggtcagcagccgcctcctcccccgcggaccgaggcggcgccggcggccggacagcccgtggagagcttcccgctggacttcacggccgtggagggtaacatggacagcttcatggcgcaagtcaagagcctggcgcagtccctgtacccctgctccgcgcagcagctcaacgaggacctgcgcctgcacctcctactcaacacctcggtgacctgcaacgacggcagccccgccggctactacctgaaggagtccaggggcagccggcggtggctcctcttcctggaaggcggctggtactgcttcaaccgcgagaactgcgactccagatacgacaccatgcggcgcctcatgagctcccgggactggccgcgcactcgcacaggcacagggatcctgtcctcacagccggaggagaacccctactggtggaacgcaaacatggtcttcatcccctactgctccagtgatgtttggagcggggcttcatccaagtctgagaagaacgagtacgccttcatgggcgccctcatcatccaggaggtggtgcgggagcttctgggcagagggctgagcggggccaaggtgctgctgctggccgggagcagcgcggggggcaccggggtgctcctgaatgtggaccgtgtggctgagcagctggagaagctgggctacccagccatccaggtgcgaggcctggctgactccggctggttcctggacaacaagcagtatcgccacacagactgcgtcgacacgatcacgtgcgcgcccacggaggccatccgccgtggcatcaggtactggaacggggtggtcccggagcgctgccgacgccagttccaggagggcgaggagtggaactgcttctttggctacaaggtctacccgaccctgcgctgccctgtgttcgtggtgcagtggctgtttgacgaggcacagctgacggtggacaacgtgcacctgacggggcagccggtgcaggagggcctgcggctgtacatccagaacctcggccgcgagctgcgccacacactcaaggacgtgccggccagctttgcccccgcctgcctctcccatgagatcatcatccggagccactggacggatgtccaggtgaaggggacgtcgctgccccgagcactgcactgctgggacaggagcctccatgacagccacaaggccagcaagacccccctcaagggctgccccgtccacctggtggacagctgcccctggccccactgcaacccctcatgccccaccgtccgagaccagttcacggggcaagagatgaacgtggcccagttcctcatgcacatgggcttcgacatgcagacggtggcccagccgcagggactggagcccagtgagctgctggggatgctgagcaacggaagctag

SEQ ID NO: 2 - protein, 496 aa, Homo sapiens
MGRGVRVLLLLSLLHCAGGSEGRKTWRRRGQQPPPPPRTEAAPAAGQPVESFPLDFTAVEGNMDSFMAQVKSLAQSLYPCSAQQLNEDLRLHLLLNTSVTCNDGSPAGYYLKESRGSRRWLLFLEGGWYCFNRENCDSRYDTMRRLMSSRDWPRTRTGTGILSSQPEENPYWWNANMVFIPYCSSDVWSGASSKSEKNEYAFMGALIIQEVVRELLGRGLSGAKVLLLAGSSAGGTGVLLNVDRVAEQLEKLGYPAIQVRGLADSGWFLDNKQYRHTDCVDTITCAPTEAIRRGIRYWNGVVPERCRRQFQEGEEWNCFFGYKVYPTLRCPVFVVQWLFDEAQLTVDNVHLTGQPVQEGLRLYIQNLGRELRHTLKDVPASFAPACLSHEIIIRSHWTDVQVKGTSLPRALHCWDRSLHDSHKASKTPLKGCPVHLVDSCPWPHCNPSCPTVRDQFTGQEMNVAQFLMHMGFDMQTVAQPQGLEPSELLGMLSNGS

SEQ ID NO: 3 - DNA, 351 bp, Mus sp.
caggtccaactgcagcagcctggggctgagcttgtgaagcctggggcttcagtgaagatgtcctgcaaggcttctggctacaccttcaccagatactggataagctgggtgaagcagaggcctggacaaggccttgagtggattggagatttttatcctggtagtggtagaaccgactacaatgagaagttcaagaccaaggccacactgactgtagacacatcctccagcacagcctacatgcagctcagcagcctgacatctgaggactctgcggtctattactgttcaagagacggtcacggcgagggtgactactggggccagggcaccactctcacagtctcctca

SEQ ID NO: 4 - protein, 117 aa, Mus sp.
QVQLQQPGAELVKPGASVKMSCKASGYTFTRYWISWVKQRPGQGLEWIGDFYPGSGRTDYNEKFKTKATLTVDTSSSTAYMQLSSLTSEDSAVYYCSRDGHGEGDYWGQGTTLTVSS

SEQ ID NO: 5 - DNA, 339 bp, Mus sp.
gatgttttgatgacccaaactccactctccctgcctgtcagtcttggagatcaagcctccatctcttgcagatctagtcagaacattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaacgg

SEQ ID NO: 6 - protein, 113 aa, Mus sp.
DVLMTQTPLSLPVSLGDQASISCRSSQNIVHSNGNTYLEWYLQKPGQSPKLLIYKVSNRFSGVPDRFSGSGSGTDFTLKISRVEAEDLGVYYCFQGSHVPYTFGGGTKLEIKR

SEQ ID NO: 7 - DNA, 354 bp, Mus sp.
caggtccaactgcagcagcctgggactgagcttgtgaagcctggggcttcagtgaagctgtcctgcaaggcttctggctacaccttcaccacctactggatgcactgggttaagcagaggcctggacgaggccttgagtggattggaaggattgatcctaatcgtggtggttctaagttcaatgagaagttcaagaccaaggccacactgactgtagacaaaccctccagcacagcctacatgcagctccgcagcctgacatctgaggactctgcggtctattattgtgcaagagattcttacgggccctacttagactactggggccaaggcaccactctcacagtctcctca

SEQ ID NO: 8 - protein, 118 aa, Mus sp.
QVQLQQPGTELVKPGASVKLSCKASGYTFTTYWMHWVKQRPGRGLEWIGRIDPNRGGSKFNEKFKTKATLTVDKPSSTAYMQLRSLTSEDSAVYYCARDSYGPYLDYWGQGTTLTVSS

SEQ ID NO: 9 - DNA, 336 bp, Mus sp.
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctttggtgcatccaatctagaatctgggatcccagccaggtttagtggcagtgggtctgggacagacttcaccctcaacatccatccggtggaggaggaggatgctgcaacctattactgtcagcaaagtaaagaggatcctccgacgttcggtggaggcaccaagctggaaatcaaacgg

SEQ ID NO: 10 - protein, 112 aa, Mus sp.
DIVLTQSPASLAVSLGQRATISCKASQSVDYDGDSYMNWYQQKPGQPPKLLIFGASNLESGIPARFSGSGSGTDFTLNIHPVEEEDAATYYCQQSKEDPPTFGGGTKLEIKR

SEQ ID NO: 11 - DNA, 339 bp, Mus sp.
gaggtccagctgcagcagtctggacctgacctggtgaagcctggggcttcagtgaagatatcctgcaaggcttctggttattcattcactggctactacatacactgggtgaagcagagccatggaaagagccttgagtggattggacgtgttaatcctaacaatggtggtactacctacaaccagaagttcaagggcaaggccataataactgtagacaagtcatccaacacagcctacatggagttccgcagcctgacatctgaggactctgcggtctattattgtacaaatgaagggacgttctggggccaaggcaccactctcacagtctcctca

SEQ ID NO: 12 - protein, 113 aa, Mus sp.
EVQLQQSGPDLVKPGASVKISCKASGYSFTGYYIHWVKQSHGKSLEWIGRVNPNNGGTTYNQKFKGKAIITVDKSSNTAYMEFRSLTSEDSAVYYCTNEGTFWGQGTTLTVSS

SEQ ID NO: 13 - DNA, 336 bp, Mus sp.
gacattgtactgacccaatctccagcttctttggctgtgtctccagggcagagggccaccatctcctgcaaggccagccaaagtgtagattatgatggtgatagttatatgaactggtaccaacagaaaccaggacagccacccaaactcctcatctattctgcatccgatctagaatctgggatcccagccaggtttattggcagtgggtctgggacagacttcaccctcaacatccatcctgtggaggaggaggatgctgcaacctattactgtcaccaaagtaatgaggatccattcacgttcggctcggggacaaagttggagataaaacgg

SEQ ID NO: 14 - protein, 112 aa, Mus sp.
DIVLTQSPASLAVSPGQRATISCKASQSVDYDGDSYMNWYQQKPGQPPKLLIYSASDLESGIPARFIGSGSGTDFTLNIHPVEEEDAATYYCHQSNEDPFTFGSGTKLEIKR

SEQ ID NO: 15 - DNA, 360 bp, Mus sp.
caggtccaactgctgcagcctgggtctgtgctggtgaggcctggagcttcagtgaagctgtcctgcaaggcttctggctacacattcaccagctactggatgcactgggtgaagcggaggcctggacaaggccttgagtggattggagagatttatcctaataatggtcggactacctacaatgagaagttcaagggcaaggccacactgactgtagacacatcctccagcacagcctacgtggatctcagcagcctgacatctgaggactctgcggtctattcctgtgcaagagggctctactatgattacgactggtttgcttactggggccaagggactctggtcactgtctctgca

SEQ ID NO: 16 - protein, 120 aa, Mus sp.
QVQLLQPGSVLVRPGASVKLSCKASGYTFTSYWMHWVKRRPGQGLEWIGEIYPNNGRTTYNEKFKGKATLTVDTSSSTAYVDLSSLTSEDSAVYSCARGLYYDYDWFAYWGQGTLVTVSA

SEQ ID NO: 17 - DNA, 342 bp, Mus sp.
gacattatgatgtcacagtctccatcctccctagctgtgtcagttggagagaaggttactctgagctgcaagtccagtcagagccttttatatagtagcaatcaaaagaactacgtggcctggtaccagcagaaaccagggcagtctcctaaagtgctgatttactgggcatccactagggaatctggggtccctgatcgcttcacaggcagtggatctgggacagatttcactctcaccatcagcagtgtgaaggctgaggacctggcagtttatcactgtcagcaatattatcgctatccgtacacgttcggaggggggaccaagctggaaataaaacgg

SEQ ID NO: 18 - protein, 114 aa, Mus sp.
DIMMSQSPSSLAVSVGEKVTLSCKSSQSLLYSSNQKNYVAWYQQKPGQSPKVLIYWASTRESGVPDRFTGSGSGTDFTLTISSVKAEDLAVYHCQQYYRYPYTFGGGTKLEIKR

SEQ ID NO: 19 - DNA, 354 bp, Mus sp.
caggtgcagctgaaggagtcaggacctggcctaatggcgccctcacagagcctgtccatcacatgcactgtctcagggttctcgttaaccgactatggtgtaagctggattcgccagcctcctggaaagggtctggagtggctgggagtaatttgggctggtggaagcacagactataattcaactctcaaatccagactgagcatcagcaaggacaactccaagagtcaagttttcataaaaatgaacagtctgcaaactgatgacacagccatgtactactgtgccaaacagaataggtacgacgggatctttgactactggggccaaggcaccactctcacagtctcctca

SEQ ID NO: 20 - protein, 118 aa, Mus sp.
QVQLKESGPGLMAPSQSLSITCTVSGFSLTDYGVSWIRQPPGKGLEWLGVIWAGGSTDYNSTLKSRLSISKDNSKSQVFIKMNSLQTDDTAMYYCAKQNRYDGIFDYWGQGTTLTVSS

SEQ ID NO: 21 - DNA, 345 bp, Mus sp.
gacattgtgatgtcacagtctccatcctccctagctgtgtcagttggagagaaggttactatgacctgcacgtccagtcagagccttttatttagtagcaatcaaaagaactacttggcctggtaccagcagaaaccagggcagtctcctaaactgctggtttcctgggcatccactagggaatctggggtccctgatcgcttcacaggcagtggatctgggacagatttcactctcaccatcagcagtgtggaggctgaagacctggcagtttattactgtcagcaatattatagctatcctccgtggacgttcggtggaggcaccaagctggaaatcaaacgg

SEQ ID NO: 22 - protein, 115 aa, Mus sp.
DIVMSQSPSSLAVSVGEKVTMTCTSSQSLLFSSNQKNYLAWYQQKPGQSPKLLVSWASTRESGVPDRFTGSGSGTDFTLTISSVEAEDLAVYYCQQYYSYPPWTFGGGTKLEIKR

SEQ ID NO: 23 - DNA, 354 bp, Mus sp.
caggtccaactgcagcagcctggggctgaactggtgaagcctggggcttcagtgaagctgtcctgcaaggcttctggctacaccttcaccaactactggatacactgggtgaagcagaggcctggacaaggccttgagtggattggagagattaatcctagcaacggtcgtactaactacaatgagaacttcacgagcaaggccacactgactgtagacaaatcctccagcacagcctacatgcatctcagcagcctgacatctgaggactctgcggtccattactgtgctaggaattatggtaactaccggtttgcttactggggccaagggactctggtcactgtctctgca

SEQ ID NO: 24 - protein, 118 aa, Mus sp.
QVQLQQPGAELVKPGASVKLSCKASGYTFTNYWIHWVKQRPGQGLEWIGEINPSNGRTNYNENFTSKATLTVDKSSSTAYMHLSSLTSEDSAVHYCARNYGNYRFAYWGQGTLVTVSA

SEQ ID NO: 25 - DNA, 321 bp, Mus sp.
gacatccagatgacacaatcttcatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagtactccgctcacgttcggtgctgggaccaagctggagctgaaa

SEQ ID NO: 26 - protein, 107 aa, Mus sp.
DIQMTQSSSYLSVSLGGRVTITCKASDHINNWLAWYQQKPGNAPRLLISGATSLETGVPSRFSGSGSGKDYTLSITSLQTEDVATYYCQQYWSTPLTFGAGTKLELK

SEQ ID NO: 27 - DNA, 357 bp, Mus sp.
caggtgcagctgaagcagtcaggacctggcctagtgcagccctcacagagcctgtccatcacctgcacagtctctggtttctcattaactaattatggtgtacactgggttcgccagtctccaggaaagggtctggagtggctgggagtgatgtggagtggtggaagcacagactataatgtagctttcatatccagactgagcatcagcaaggacaattccaagagccaagttttctttaaaatgaacagtctgcaagctgatgacacagccatatattactgtgccagaagcccctatagtaattatgactactttgactactggggccgaggcaccactctcacagtctcctca

SEQ ID NO: 28 - protein, 119 aa, Mus sp.
QVQLKQSGPGLVQPSQSLSITCTVSGFSLTNYGVHWVRQSPGKGLEWLGVMWSGGSTDYNVAFISRLSISKDNSKSQVFFKMNSLQADDTAIYYCARSPYSNYDYFDYWGRGTTLTVSS

SEQ ID NO: 29 - DNA, 321 bp, Mus sp.
gacatccagatgaaccagtctccatccagtctgtctgcatcccttggagacacaattaccatcacttgccatgccagtcagaacattaatgtttggttaagctggtaccagcagaaaccaggaaattttcctaaacttttgatctttaaggcttccaacttgcacacaggcgtcccatcaaggtttagtggcagtggatctggaacaggtttcacattaaccatcagcaacctgcagcctgaagacattgccacttactactgtcaacagggtcaaagttatcctctgacgttcggtggaggcacccagctggagatcaaa

SEQ ID NO: 30 - protein, 107 aa, Mus sp.
DIQMNQSPSSLSASLGDTITITCHASQNINVWLSWYQQKPGNFPKLLIFKASNLHTGVPSRFSGSGSGTGFTLTISNLQPEDIATYYCQQGQSYPLTFGGGTQLEIK

SEQ ID NO: 31 - DNA, 357 bp, Mus sp.
caggttcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaatctcaaggggaaagccacactgactgcagacagatcctccagcacagcctacatccagctcagcagcctaacatctgaggactctgcggtctatttctgtgcaagacagctcgggctaccttatgctatggactactggggtcaaggaacctcagtcaccgtctcctca

SEQ ID NO: 32 - protein, 119 aa, Mus sp.
QVQLQQSGAELVRPGSSVKISCKASGYAFSSYWMNWVKQRPGQGLEWIGQIYPGDGDTNYNGNLKGKATLTADRSSSTAYIQLSSLTSEDSAVYFCARQLGLPYAMDYWGQGTSVTVSS

SEQ ID NO: 33 - DNA, 324 bp, Mus sp.
gacatcctgatgacccaatctccatcctccatgtctgtatctctgggagacacagtcagcatcacttgccatgcaagtcaggacattagcagtaatatagggtggttgcagcagaaaccagggaaatcatttaagggcctgatctatcatggaaccaacttggaagatggagttccatcaaggttcagtggcagtggatctggagcagattattctctcaccatcaccagcctggaatctgaagattttgcagactattactgtgtacagtatgctcagtttccgtacacgttcggaggggggaccaagctggaaataaaacgg

SEQ ID NO: 34 - protein, 108 aa, Mus sp.
DILMTQSPSSMSVSLGDTVSITCHASQDISSNIGWLQQKPGKSFKGLIYHGTNLEDGVPSRFSGSGSGADYSLTITSLESEDFADYYCVQYAQFPYTFGGGTKLEIKR

SEQ ID NO: 35 - DNA, 357 bp, Mus sp.
gacgtgaagctggtggagtctggggaaggcttagtgaagcctggagggtccctgaaactctcctgtgcagcctctggattcactttcagtaactatgccatgtcttgggttcgccagactccagagaagaggctggagtgggtcgcatacattagtagtggtggtgattacatctactatgcagacactgtgaagggccgattcaccatctccagagacaatgccaggaacaccctgtacctgcaaatgagcagtctgaagtctgaggacacagccatgtattactgtacaagagaggatggttattactctactatggactactggggtcaaggaacctcagtcaccgtctcctca

SEQ ID NO: 36 - protein, 119 aa, Mus sp.
DVKLVESGEGLVKPGGSLKLSCAASGFTFSNYAMSWVRQTPEKRLEWVAYISSGGDYIYYADTVKGRFTISRDNARNTLYLQMSSLKSEDTAMYYCTREDGYYSTMDYWGQGTSVTVSS

SEQ ID NO: 37 - DNA, 324 bp, Mus sp.
gacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcagccagattggtagatggggtcccatcgaggttcagtggcagtggatctgggcaagattattctctcaccatcagcagcctggagtatgaagatatgggagtttattattgtctacagtatgatgagtttccgtacacgttcggaggggggaccaagctggaaataaaacgg

SEQ ID NO: 38 - protein, 108 aa, Mus sp.
DIKMTQSPSSMYASLGERVTITCKASQDINSYLSWFQQKPGKSPKTLIYRAARLVDGVPSRFSGSGSGQDYSLTISSLEYEDMGVYYCLQYDEFPYTFGGGTKLEIKR

SEQ ID NO: 39 - DNA, 351 bp, Mus sp.
caggtgcaactgaaggagtcaggacctggcctggtggcgccctcacagagcctgtccatcacttgcactgtctcggaattttcattaaatagctatggtgttcaatgggttcgccagcctccaggaaagggtctggagtggctgggagtaatatgggctggtggaatcacaaattataattcggctctcatgttcagactgagcatcagcaaagacaactccaagagccaagttttcttaaaaatgaacagtctgcaaactgatgacacagccatgtactactgtgccaggagttataattacgacgtcgtgtttgcttactggggccaagggactttggcccctgtctct

SEQ ID NO: 40 - protein, 117 aa, Mus sp.
QVQLKESGPGLVAPSQSLSITCTVSEFSLNSYGVQWVRQPPGKGLEWLGVIWAGGITNYNSALMFRLSISKDNSKSQVFLKMNSLQTDDTAMYYCARSYNYDVVFAYWGQGTLAPVS

SEQ ID NO: 41 - DNA, 345 bp, Mus sp.
gacattgtgatgtcacagtctccatcctccctagctgtgtcagttggagagaaggttacaatgagttgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctccgtacacgttcggaggggggaccaagctggaaataaaacgg

SEQ ID NO: 42 - protein, 115 aa, Mus sp.
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPPYTFGGGTKLEIKR

SEQ ID NO: 43 - DNA, 351 bp, Mus sp.
caggtccaattgcagcagtctggggctgagctggtgaggcctggggcttcagtgaagctgtcctgcaaggcttctggcttcacgttcatcagctactggatgaactgggttaagcagaggcctgagcaaggccttgagtggattggaaggattgatccttacgatagtgaaactcactacaatcaaattttcaaggacaaggccatattgactgtagacaagtcctccagcacagcctacatgcaactcagcagcctgacatctgaggactctgcggtctattactgtgcaagacgcggcctgcactactttgactactggggccaaggcaccactctcacagtctcctca

SEQ ID NO: 44 - protein, 117 aa, Mus sp.
QVQLQQSGAELVRPGASVKLSCKASGFTFISYWMNWVKQRPEQGLEWIGRIDPYDSETHYNQIFKDKAILTVDKSSSTAYMQLSSLTSEDSAVYYCARRGLHYFDYWGQGTTLTVSS

SEQ ID NO: 45 - DNA, 334 bp, Mus sp.
atgttttgatgacccaaactccactctccctgcctgtcagtcttggagatcaagcctcctctcttgcagatctagtcagaacattgtacatagtaatggaaacacctatttagaatggacctgcagaaaccaggccagtctccaaagctcctgatctacaaagtttccaaccgatttctggggtcccagacagggtcagtggcagtggatcagggacagatttcacactcaagatcgcagagtggaggctgaggatctgggagtttattactgctttcaaggttcacatgttccattcacgttcggctcggggacaaagttggaaataaaacgg

SEQ ID NO: 46 - protein, 113 aa, Mus sp.
DVLMTQTPLSLPVSLGDQASISCRSSQNIVHSNGNTYLEWYLQKPGQSPKLLIYKVSNRFSGVPDRVSGSGSGTDFTLKISRVEAEDLGVYYCFQGSHVPFTFGSGTKLEIKR

SEQ ID NO: 47 - DNA, 351 bp, Mus sp.
caggtccagctgcagcaatctggacctgaactggtgaagcctggggcttcagtgaagatatcctgcaaggcttctggctatatcttcacaaacttctatatacattgggtgaaacagaggcctggacagggccttgagtggattggatatatttatcctagagatggtaatactaattacaatgagaacttcaagggcaaggccacactgactgcggacacatcctccaccacagcctacatgcagctcagcagcctgacatctgaggactctgcagtctatttctgtgcaagagggggctgggacgactggtttgcttactggggccaagggactctggtcactgtctct

SEQ ID NO: 48 - protein, 117 aa, Mus sp.
QVQLQQSGPELVKPGASVKISCKASGYIFTNFYIHWVKQRPGQGLEWIGYIYPRDGNTNYNENFKGKATLTADTSSTTAYMQLSSLTSEDSAVYFCARGGWDDWFAYWGQGTLVTVS

SEQ ID NO: 49 - DNA, 339 bp, Mus sp.
gatgttttgatgacccaaactccactctccctgcctgtcagtcttggagatcaagcctccatctcttgcagatctagtcagaccattgttcacagaagtggaagcacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaacgg

SEQ ID NO: 50 - protein, 113 aa, Mus sp.
DVLMTQTPLSLPVSLGDQASISCRSSQTIVHRSGSTYLEWYLQKPGQSPKLLIYKVSNRFSGVPDRFSGSGSGTDFTLKISRVEAEDLGVYYCFQGSHVPLTFGAGTKLELKR

SEQ ID NO: 51 - DNA, 357 bp, Mus sp.
caggtccaactgcagcagcctggggctgagctggtgaggcctgggacttcagtgaagttgtcctgcaaggcttctggctacaccttcaccagctactggatgcactgggtaaagcagaggcctggacaaggccttgagtggatcggagtgattgatccctctgacagttatagtaactacaatcaaaagttcaagggcaaggccacattgactgtagacacatcctccaacacagcctacatgcagctcagcagcctgacatctgaggactctgcggtctattactgtgcaagatatggtgggacgggctatggtatggactactggggtcaaggaacctcagtcaccgtctcctca

SEQ ID NO: 52 - protein, 119 aa, Mus sp.
QVQLQQPGAELVRPGTSVKLSCKASGYTFTSYWMHWVKQRPGQGLEWIGVIDPSDSYSNYNQKFKGKATLTVDTSSNTAYMQLSSLTSEDSAVYYCARYGGTGYGMDYWGQGTSVTVSS

SEQ ID NO: 53 - DNA, 336 bp, Mus sp.
gactttgtgctgacccaatctccagcttctttggctgtgtctctaggacagagggccaccatatcctgccaagccagcgaaggtgtcagttttgctggttcaagtttaatgcactggtaccaacagaaaccaggacagccacccaaactcctcatctatcgtgcatccaacctagaatctggagtccctgccaggttcagtggcagtgggtctgagtcagacttcactctcaccatcgatcctgtggaggaagatgatgctgcaatgtattactgtatgcaaagtatggaagatccattcacgttcggctcggggacaaagttggaaataaaacgg

SEQ ID NO: 54 - protein, 112 aa, Mus sp.
DFVLTQSPASLAVSLGQRATISCQASEGVSFAGSSLMHWYQQKPGQPPKLLIYRASNLESGVPARFSGSGSESDFTLTIDPVEEDDAAMYYCMQSMEDPFTFGSGTKLEIKR

SEQ ID NO: 55 - DNA, 357 bp, Mus sp.
gatgtgcagctggtggagtctgggggaggcttagtgcagcctggagggtcccggaaaatctcctgtgcagcctctggattcactttcagtagctatggaatgcactgggttcgtcaggctccagagaaggggctggagtgggtcgcatatattagtagtagtactgtgaccacctactatgcggacacagtgaagggccgattcaccatctccagagacaatgccaacaacaccctgttcctgcaaatgaccagtctaaggtctgaggacacggccatgtattactgttcaagacaaaactgggacggagggtacttcgatgtctggggcgcagggaccacggtcaccgtctcctca

SEQ ID NO: 56 - protein, 119 aa, Mus sp.
DVQLVESGGGLVQPGGSRKISCAASGFTFSSYGMHWVRQAPEKGLEWVAYISSSTVTTYYADTVKGRFTISRDNANNTLFLQMTSLRSEDTAMYYCSRQNWDGGYFDVWGAGTTVTVSS

SEQ ID NO: 57 - DNA, 336 bp, Mus sp.
gacattgtgctgacacagtctcttgcttccttagctgtttctctggggcagagggccaccatctcatgcagggccagcaaaagtgtcagttcttctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggcgcttccgctcacgttcggtgctgggaccaagctggagctgaaacgg

SEQ ID NO: 58 - protein, 112 aa, Mus sp.
DIVLTQSLASLAVSLGQRATISCRASKSVSSSGYSYMHWYQQKPGQPPKLLIYLASNLESGVPARFSGSGSGTDFTLNIHPVEEEDAATYYCQHSRALPLTFGAGTKLELKR

SEQ ID NO: 59 - DNA, 354 bp, Mus sp.
cagatccagctgcagcagtctggacctgagctggtgaagcctggggcttcagtgaagatatcctgcaaggcttctggctacaggttcactgactactatataaactgggtgaagcagaagcctggacagggacttgagtggattggatggatttatcctggcaacggtaatactaagtacaatgagaagttcaagggcacggccacattgactgtagacacatcctccagcacagcctacatgcaactcagcagcctgacatctgaggacactgctgtctatttctgtgcaaggtatggtaacttactggacctggactactggggtcaaggaacctcagtcaccgtctcctca

SEQ ID NO: 60 - protein, 118 aa, Mus sp.
QIQLQQSGPELVKPGASVKISCKASGYRFTDYYINWVKQKPGQGLEWIGWIYPGNGNTKYNEKFKGTATLTVDTSSSTAYMQLSSLTSEDTAVYFCARYGNLLDLDYWGQGTSVTVSS

SEQ ID NO: 61 - DNA, 324 bp, Mus sp.
gacatccagatgactcagtctccagcctccctatctgtatctgtgggagaaactgtcaccatcacatgtcgagcaagtgagaatatttacattaatttagcatggtatcaacagaaacagggaaaatctcctcggctcctggtctatgctgcaacaaacttagcagatggtgtgccatcgaggttcagtggcagtggatcaggcacacagttttccctcaagatcaacagcctgcagtctgaagattttgggacttattactgtcaacatttttggggtactccgtacacgttcggaggggggaccaagctggaaataaaacgg

SEQ ID NO: 62 - protein, 108 aa, Mus sp.
DIQMTQSPASLSVSVGETVTITCRASENIYINLAWYQQKQGKSPRLLVYAATNLADGVPSRFSGSGSGTQFSLKINSLQSEDFGTYYCQHFWGTPYTFGGGTKLEIKR

SEQ ID NO: 63 - DNA, 342 bp, Mus sp.
gaggtccagctgcaacagtctggacctgagctggtgaagcctggggcttcagtgaagatatcctgcaagacttctggatacacattcactgaatacaccatgcactgggtgaagcagagccatggaaagagccttgagtggattggaggtattaatcctaacaatggtgttactagctacaaccagaagttcaagggcaaggccacattggctgtagacaagtcctccaccacagcctacatggagctccgcagcctgacatctgagggttctgcagtctattactgtgcaagaggggcctttgactactggggccaaggcaccactctcacagtctcctca

SEQ ID NO: 64 - protein, 115 aa, Mus sp.
EVQLQQSGPELVKPGASVKISCKTSGYTFTEYTMHWVKQSHGKSLEWIGGINPNNGVTSYNQKFKGKATLAVDKSSTTAYMELRSLTSEGSAVYYCARGAFDYWGQGTTLTVSSA

SEQ ID NO: 65 - DNA, 339 bp, Mus sp.
gatgttgtgatgacccagactccactcactttgtcggttaccattggacaaccagcctctatctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtgggtcaggaacagattttacactgaaaatcagcagagtggaggctgaggatttgggagtttattactgcgtgcaaggtacacattttccgtacacgttcggaggggggaccaagctggaaataaaacgg

SEQ ID NO: 66 - protein, 116 aa, Mus sp.
TNGDVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPYTFGGGTKLEIKR

SEQ ID NO: 67 - DNA, 351 bp, Mus sp.
caagtaactctaaaagagtctggccctgggatattgaagccctcacagaccctcagtctgacttgttctttctctgggttttcactgagcacttctggtatgggtgtaggctggattcgtcagccttcagggaagggtctggagtggctggcactcatttggtgggatgatgataaatactataacccatccctgaagagccagctcacaatctccaaggatacctccagaaaccaggtattcctcaagatcaccagtgtggacactgcagatactgccacttactactgtgttcgaacttatggttactatgagagctggggccaagggactctggtcactgtctctgca

SEQ ID NO: 68 - protein, 117 aa, Mus sp.
QVTLKESGPGILKPSQTLSLTCSFSGFSLSTSGMGVGWIRQPSGKGLEWLALIWWDDDKYYNPSLKSQLTISKDTSRNQVFLKITSVDTADTATYYCVRTYGYYESWGQGTLVTVSA

SEQ ID NO: 69 - DNA, 339 bp, Mus sp.
gatgttgtgatgccccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcgcactgaaaatcagcagagtggaggctgaggatttgggagtttattattgctggcaaggtacacattttcctcagacgttcggtggaggcaccaagctggaaatcaaacgg

SEQ ID NO: 70 - protein, 113 aa, Mus sp.
DVVMPQTPLTLSVTIGQPASISCKSSQSLLDSDGKTYLNWLLQRPGQSPKRLIYLVSKLDSGVPDRFTGSGSGTDFALKISRVEAEDLGVYYCWQGTHFPQTFGGGTKLEIKR

SEQ ID NO: 71 - DNA, 354 bp, Mus sp.
gaggtccagctgcaacagtctggacctgagttggtgaagcctggggcttcagtgaagatgtcctgcaaggcttctggctacacattcactgactactacatgcactgggtgaagcagagccatggaaagagccttgagtggattggatatatttatcctaacaatggtggtaatggctacaaccagaagttcaagggcagggccacattgactgttgacaagtcctccagcacagcctacatggagctccgcagcctgacatctgaggactctgcagtctattactgtgcaagacggggggccactgggttctttgactactggggccaaggcaccactctcacagtctcctca

SEQ ID NO: 72 - protein, 118 aa, Mus sp.
EVQLQQSGPELVKPGASVKMSCKASGYTFTDYYMHWVKQSHGKSLEWIGYIYPNNGGNGYNQKFKGRATLTVDKSSSTAYMELRSLTSEDSAVYYCARRGATGFFDYWGQGTTLTVSS

SEQ ID NO: 73 - DNA, 321 bp, Mus sp.
gacatccagatgactcagtctccagactccctatctgcatctgtgggagaaactgtcaccatcacatgtcgagcaagtgaaaatatttacagtactttagcatggtatcagcagacaccgggagaatctcctcagctcctggtctatactggaacaaatttagcagataatataccatcaaggttcagtggcagtggatcaggcacacagtattctctgaagatcaaaagtctgcagcctgaggattttgggagttattactgtcaacatttttatggtacggtcacgttcggtgctgggaccaagctggaactgaaacgg

SEQ ID NO: 74 - protein, 107 aa, Mus sp.
DIQMTQSPDSLSASVGETVTITCRASENIYSTLAWYQQTPGESPQLLVYTGTNLADNIPSRFSGSGSGTQYSLKIKSLQPEDFGSYYCQHFYGTVTFGAGTKLELKR

SEQ ID NO: 75 - DNA, 348 bp, Mus sp.
caggttcagctgcagcagtctggggatgagctggtgaggcctgggtcctcagtgaagatttcctgcaaggcctctggctatgcattcagtagctactggatgaactgggtgaagcagaggcctggacagggtcttgagtggattggacagatttatcctggagatggtgatactaagtacaatggaaagttcaagggtaaagccacactgactgcagacaaatcctccagcacagcctacatgcagctcagcagcctaacatctgcggactctgcggtctatttctgtgcggggccccactggtacctcgatgtctggggcgcagggaccacggtcaccgtctcctca

SEQ ID NO: 76 - protein, 116 aa, Mus sp.
QVQLQQSGDELVRPGSSVKISCKASGYAFSSYWMNWVKQRPGQGLEWIGQIYPGDGDTKYNGKFKGKATLTADKSSSTAYMQLSSLTSADSAVYFCAGPHWYLDVWGAGTTVTVSS

SEQ ID NO: 77 - DNA, 324 bp, Mus sp.
gatatccagatgacacagactacatcctccctgtctgcctctctgggagacagagtcaccatcaattgcagggcaagtcaggacattagcaattatttaaactggtatcagcagaaaccagatggaactattaaactcctgatctactacacatcaagattacactcaggagtcccatcaaggttcagtggcagtgggtctggaacagattattctctcaccattagcaacctggagcaagaagatattgccacttacttttgccaacagactaatacgcttcctcggacgttcggtggaggcaccaagctggaaatcaaacgg

SEQ ID NO: 78 - protein, 108 aa, Mus sp.
DIQMTQTTSSLSASLGDRVTINCRASQDISNYLNWYQQKPDGTIKLLIYYTSRLHSGVPSRFSGSGSGTDYSLTISNLEQEDIATYFCQQTNTLPRTFGGGTKLEIKR

SEQ ID NO: 79 - DNA, 336 bp, Mus sp.
caggtccaactgcagcagcctggggctgaaattgtgaggcctggggcttcagtgaaactgtcctgcaaggcttctggctacacctttaccgactattggatgaactgggtgaagcagaggcctggacaaggccttgagtggatcggagcaattgatccttctatttcttatactacctacaatcaaaagttcaagggcaaggccacattgactgtagacacatcctccagctcagcctacatgcagctcagcagcctgacctctgaggactctgcgatctatttctgtgcaagagatggcgactggggccaagggactctggtcgctgtctctgca

SEQ ID NO: 80 - protein, 112 aa, Mus sp.
QVQLQQPGAEIVRPGASVKLSCKASGYTFTDYWMNWVKQRPGQGLEWIGAIDPSISYTTYNQKFKGKATLTVDTSSSSAYMQLSSLTSEDSAIYFCARDGDWGQGTLVAVSA

SEQ ID NO: 81 - DNA, 339 bp, Mus sp.
gatgttgtgatgacccagactccactcactttgtcggttgccattggacaaccagcctccatctcttgcaagtcaagtcagagcctcttagatagtgatggaaagacatatttgaattggttgttacagaggccaggccagtctccaaagcgcctaatctatctggtgtctaaacttgactctggagtccctgacaggttcactgccagtggatcagggacagatttcacactgaaaatcagcagagtggaggctgaggatttgggagtttattattgctggcaaggtacacattttcctcagacgttcggtggaggcaccacgctggaaatcaaacgg

SEQ ID NO: 82 - protein, 113 aa, Mus sp.
DVVMTQTPLTLSVAIGQPASISCKSSQSLLDSDGKTYLNWLLQRPGQSPKRLIYLVSKLDSGVPDRFTASGSGTDFTLKISRVEAEDLGVYYCWQGTHFPQTFGGGTTLEIKR

SEQ ID NO: 83 - DNA, 366 bp, Mus sp.
gacgtgaagctggtggagtctgggggagtcttagtgaagcctggagggtccctgaaactctcctgtgcagcctctggattcactttcagtagctataccatgtcttgggttcgccagactccggagaagaggctggagtgggtcgcaaccattagtagtggtggtagttacacctactattcagacagtgtgaagggccgattcaccatctccagagacaatgccaagaacaccctgtacctgcaaatgagcagtctgaagtctgaggacacagccatgtattactgtacaagagagggtgatggttactacccctactatgctatggactactggggtcaaggaacctcagtcaccgtctcctca

SEQ ID NO: 84 - protein, 122 aa, Mus sp.
DVKLVESGGVLVKPGGSLKLSCAASGFTFSSYTMSWVRQTPEKRLEWVATISSGGSYTYYSDSVKGRFTISRDNAKNTLYLQMSSLKSEDTAMYYCTREGDGYYPYYAMDYWGQGTSVTVSS

SEQ ID NO: 85 - DNA, 339 bp, Mus sp.
gatgttgtgatgacccaaactccactctccctgcctgtcagtcttggagatcaagcctccatctcttgcagatctagtcagagccttgtacacagtaatggaaacacctatttacactggtacctgcagaagccaggccagtctccaaaggtcctgatctacaaagtttccaaccgattttctggggtcccagacaggttcagtggcagtgggtcagggacagatttcacactcaagatcagcagagtggaggctgaggatctgggagtttatttctgctctcaaattacacatgttccgctcacgttcggtgctgggaccaagctggagctgaaacgg

SEQ ID NO: 86 - protein, 113 aa, Mus sp.
DVVMTQTPLSLPVSLGDQASISCRSSQSLVHSNGNTYLHWYLQKPGQSPKVLIYKVSNRFSGVPDRFSGSGSGTDFTLKISRVEAEDLGVYFCSQITHVPLTFGAGTKLELKR

SEQ ID NO: 87 - DNA, 363 bp, Mus sp.
caggtacagctgaaggagtcaggacctggcctggtggcgccctcacagagcctgtccatcacttgcactgtctctggattttcattaatcaactttggtgtacagtggattcgccagcctccaggaaagggtctggagtggctgggagtaatatgggctggtggaagaataaaatataattcgactctcatgtccagactgagcatcagcaaagacaactccaagagccaagttttcttaaaaatgaacagtctgcaaactgatgacacagccatgtattactgtgccagagatttgggactcaactgggaccccccctggtttgcttactggggccaagggactctggtcactgtctctgca

SEQ ID NO: 88 - protein, 121 aa, Mus sp.
QVQLKESGPGLVAPSQSLSITCTVSGFSLINFGVQWIRQPPGKGLEWLGVIWAGGRIKYNSTLMSRLSISKDNSKSQVFLKMNSLQTDDTAMYYCARDLGLNWDPPWFAYWGQGTLVTVSA

SEQ ID NO: 89 - DNA, 339 bp, Mus sp.
gacattgtgatgtcacagtctccatcctccctagctgtgtcagttggagagaaggttactatgaggtgtaagtccagtcagagccttttatataatagcaatcaaaagaactacttggcctggtaccagcagaaaccagggcagtctcctaaactgctgatttactgggcatccactagggaatctggggtccctgatcgcttcacaggcagtggatctgggacagatttcactctcatcatcagcagtgtgaaggctgaagacctggcagtttattactgtcagcaatatgatagctatcctcccacgttcggaggggggaccaagctggaaataaga

SEQ ID NO: 90 - protein, 113 aa, Mus sp.
DIVMSQSPSSLAVSVGEKVTMRCKSSQSLLYNSNQKNYLAWYQQKPGQSPKLLIYWASTRESGVPDRFTGSGSGTDFTLIISSVKAEDLAVYYCQQYDSYPPTFGGGTKLEIR

SEQ ID NO: 91 - DNA, 363 bp, Mus sp.
gaagtgaagctggtggagtctgggggaggcttagtgaagcctggagggtccctgaaactctcctgtgcagcctctggattcgttttcagtagccatgacatgtcttgggttcgccagactccggagaagaggctggagtgggtcgcaaccattagtagtggtggtagttacaactactattcagacagtgtgaagggccgattcaccatctccagaaacaatgccaggaacaccctatacctgcaaatgagcagtctgaggtctgaggacacggccttgtattactgtgcaagacagggcgataggtacgacggctatgttatggactactggggtcaaggaacctcagtcaccgtctcctca

SEQ ID NO: 92 - protein, 121 aa, Mus sp.
EVKLVESGGGLVKPGGSLKLSCAASGFVFSSHDMSWVRQTPEKRLEWVATISSGGSYNYYSDSVKGRFTISRNNARNTLYLQMSSLRSEDTALYYCARQGDRYDGYVMDYWGQGTSVTVSS

SEQ ID NO: 93 - DNA, 327 bp, Mus sp.
caggctgttgtgactcaggaatctgcactcaccacatcacctggtgaaacagtcacactcacttgtcgctcaagtactggggctgttacaactagttactatgccaactgggtccaagaaaaaccagatcatttattcactggtctaataggtggtaccaactaccgagctccaggtgttcctgccagattctcaggctccctgattggagacaaggctgccctcaccatcacaggggcacagactgaggatgaggcaatatatttctgtgctctatggtacaacaaccattgggtgttcggtggaggaaccaaactgactgtccta

SEQ ID NO: 94 - protein, 109 aa, Mus sp.
QAVVTQESALTTSPGETVTLTCRSSTGAVTTSYYANWVQEKPDHLFTGLIGGTNYRAPGVPARFSGSLIGDKAALTITGAQTEDEAIYFCALWYNNHWVFGGGTKLTVL

SEQ ID NO: 95 - DNA, 345 bp, Mus sp.
gaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcatccctacctttagtggactactggggtcgaggaacctcagtcaccgtctcctca

SEQ ID NO: 96 - protein, 115 aa, Mus sp.
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCASLPLVDYWGRGTSVTVSS

SEQ ID NO: 97 - DNA, 324 bp, Mus sp.
gacattgtgatgacccagtctcaaaaattcatgtccacatcagtaggagacagggtcagcgtcacctgcaaggccagtcagaatgtgggtactagtgttgcctggtttcaacagagaccaggacaatctcctaaagcactgatttactcggcatcctaccggtacagtggagtccctgatcgcttcacaggcagtggatctgggacagatttcactctcaccatcagtaatgtgcagtctgaagacttggcagactatttctgtgagcaatataaaagctatccgtacacgttcggaggggggaccaggctggaaataaaacgg

SEQ ID NO: 98 - protein, 108 aa, Mus sp.
DIVMTQSQKFMSTSVGDRVSVTCKASQNVGTSVAWFQQRPGQSPKALIYSASYRYSGVPDRFTGSGSGTDFTLTISNVQSEDLADYFCEQYKSYPYTFGGGTRLEIKR

SEQ ID NO: 99 - protein, 143 aa, Macaca sp.
SEGRKTWRRRGQQPPPPPRTEAAPAAGQPVESFPLDFTAVEGNMDSFMAQVKSLAQSLYPCSAQQLNEDLRLHLLLNTSVTCNDGSPAGYYLKESRGSRRWLLFLEGGWYCFNRENCDSRYNTMRRLMSSRDWPRTRTGTGIL

SEQ ID NO: 100 - protein, 143 aa, Homo sapiens
SEGRKTWRRRGQQPPPPPRTEAAPAAGQPVESFPLDFTAVEGNMDSFMAQVKSLAQSLYPCSAQQLNEDLRLHLLLNTSVTCNDGSPAGYYLKESRGSRRWLLFLEGGWYCFNRENCDSRYDTMRRLMSSRDWPRTRTGTGIL

SEQ ID NO: 101 - protein, 150 aa, Mus sp.
SEGRKTWRRRGQQPPQPPPPPPLPQRAEVEPGAGQPVESFPLDFTAVEGNMDSFMAQVKSLAQSLYPCSAQQLNEDLRLHLLLNTSVTCNDGSPAGYYLKESKGSRRWLLFLEGGWYCFNRENCDSRYSTMRRLMSSKDWPHTRTGTGIL

SEQ ID NO: 102 - protein, 143 aa, Artificial Sequence; identical to SEQ ID NO: 99
  source: /note=“Description of Artificial Sequence Synthetic Consensus”

SEQ ID NO: 103 - protein, 8 aa, Mus sp.
GYTFTEYT

SEQ ID NO: 104 - protein, 8 aa, Mus sp.
GYTFTRYW

SEQ ID NO: 105 - protein, 8 aa, Mus sp.
GFTFSSYT

SEQ ID NO: 106 - protein, 10 aa, Mus sp.
GFSLSTSDMG

SEQ ID NO: 107 - protein, 8 aa, Mus sp.
GYTFTTYW

SEQ ID NO: 108 - protein, 8 aa, Mus sp.
GYSFTSYW

SEQ ID NO: 109 - protein, 10 aa, Mus sp.
GFSLSTSGMG

SEQ ID NO: 110 - protein, 10 aa, Mus sp.
GYSFTGYYIH

SEQ ID NO: 111 - protein, 10 aa, Mus sp.
GYTFTSYWMH

SEQ ID NO: 112 - protein, 10 aa, Mus sp.
GFSLTDYGVS

SEQ ID NO: 113 - protein, 10 aa, Mus sp.
GYTFTNYWIH

SEQ ID NO: 114 - protein, 10 aa, Mus sp.
GFSLTNYGVH

SEQ ID NO: 115 - protein, 10 aa, Mus sp.
GYAFSSYWMN

SEQ ID NO: 116 - protein, 10 aa, Mus sp.
GFTFSNYAMS

SEQ ID NO: 117 - protein, 10 aa, Mus sp.
EFSLNSYGVH

SEQ ID NO: 118 - protein, 10 aa, Mus sp.
GYSFTGYNMN

SEQ ID NO: 119 - protein, 10 aa, Mus sp.
GYTFTSYWMH

SEQ ID NO: 120 - protein, 10 aa, Mus sp.
GFTFISYWMN

SEQ ID NO: 121 - protein, 8 aa, Mus sp.
GYAFSSYW

SEQ ID NO: 122 - protein, 8 aa, Mus sp.
GFTFSSYG

SEQ ID NO: 123 - protein, 8 aa, Mus sp.
GYTFTDYW

SEQ ID NO: 124 - protein, 8 aa, Mus sp.
GYTFTNFG

SEQ ID NO: 125 - protein, 10 aa, Mus sp.
GFSLSTSGLG

SEQ ID NO: 126 - protein, 8 aa, Mus sp.
GYTFTRYW

SEQ ID NO: 127 - protein, 8 aa, Mus sp.
GYRFTDYY

SEQ ID NO: 128 - protein, 8 aa, Mus sp.
GYTFTDDN

SEQ ID NO: 129 - protein, 8 aa, Mus sp.
GFVFSSHD

SEQ ID NO: 130 - protein, 10 aa, Mus sp.
GFSLSTSGMG

SEQ ID NO: 131 - protein, 10 aa, Mus sp.
GFSLSTSGLG

SEQ ID NO: 132 - protein, 8 aa, Mus sp.
GYIFTNFY

SEQ ID NO: 133 - protein, 8 aa, Mus sp.
GYTFTDYW

SEQ ID NO: 134 - protein, 8 aa, Mus sp.
GYTFTNYW

SEQ ID NO: 135 - protein, 8 aa, Mus sp.
GYTFTSYW

SEQ ID NO: 136 - protein, 8 aa, Mus sp.
GYTFTSYY

SEQ ID NO: 137 - protein, 8 aa, Mus sp.
GFSLINFG

SEQ ID NO: 138 - protein, 8 aa, Mus sp.
GYTFTDYY

SEQ ID NO: 139 - protein, 8 aa, Mus sp.
GYTFTDYW

SEQ ID NO: 140 - protein, 8 aa, Mus sp.
GFDFSRYW

SEQ ID NO: 141 - protein, 8 aa, Mus sp.
INPNNGVT

SEQ ID NO: 142 - protein, 8 aa, Mus sp.
FYPGSGRT

SEQ ID NO: 143 - protein, 8 aa, Mus sp.
ISSGGSYT

SEQ ID NO: 144 - protein, 7 aa, Mus sp.
IWWDDDK

SEQ ID NO: 145 - protein, 8 aa, Mus sp.
IDPNRGGS

SEQ ID NO: 146 - protein, 8 aa, Mus sp.
VYPGNGDT

SEQ ID NO: 147 - protein, 7 aa, Mus sp.
IWWDDDK

SEQ ID NO: 148 - protein, 9 aa, Mus sp.
RVNPNNGGT

SEQ ID NO: 149 - protein, 8 aa, Mus sp.
IYPNNGRT

SEQ ID NO: 150 - protein, 7 aa, Mus sp.
IWAGGST

SEQ ID NO: 151 - protein, 8 aa, Mus sp.
INPSNGRT

SEQ ID NO: 152 - protein, 7 aa, Mus sp.
MWSGGST

SEQ ID NO: 153 - protein, 8 aa, Mus sp.
IYPGDGDT

SEQ ID NO: 154 - protein, 8 aa, Mus sp.
ISSGGDYI

SEQ ID NO: 155 - protein, 7 aa, Mus sp.
IWAGGIT

SEQ ID NO: 156 - protein, 8 aa, Mus sp.
IDPYYGST

SEQ ID NO: 157 - protein, 8 aa, Mus sp.
IDPSDSYS

SEQ ID NO: 158 - protein, 8 aa, Mus sp.
IDPYDSET

SEQ ID NO: 159 - protein, 8 aa, Mus sp.
IYPGDGDT

SEQ ID NO: 160 - protein, 8 aa, Mus sp.
ISSSTVTT

SEQ ID NO: 161 - protein, 8 aa, Mus sp.
IDPSYSYP

SEQ ID NO: 162 - protein, 8 aa, Mus sp.
INTYTGEP

SEQ ID NO: 163 - protein, 7 aa, Mus sp.
IWWDDNK

SEQ ID NO: 164 - protein, 8 aa, Mus sp.
INPTSDYG

SEQ ID NO: 165 - protein, 8 aa, Mus sp.
IYPGNGNT

SEQ ID NO: 166 - protein, 8 aa, Mus sp.
INPYNDGT

SEQ ID NO: 167 - protein, 8 aa, Mus sp.
ISSGGSYN

SEQ ID NO: 168 - protein, 7 aa, Mus sp.
IWWDDDE

SEQ ID NO: 169 - protein, 7 aa, Mus sp.
IWWDDSK

SEQ ID NO: 170 - protein, 8 aa, Mus sp.
IYPRDGNT

SEQ ID NO: 171 - protein, 8 aa, Mus sp.
IDTSDSYT

SEQ ID NO: 172 - protein, 8 aa, Mus sp.
IDPSDSYT

SEQ ID NO: 173 - protein, 8 aa, Mus sp.
INPSNGGA

SEQ ID NO: 174 - protein, 8 aa, Mus sp.
INPNNGRT

SEQ ID NO: 175 - protein, 7 aa, Mus sp.
IWAGGRI

SEQ ID NO: 176 - protein, 8 aa, Mus sp.
IYPNNGGN

SEQ ID NO: 177 - protein, 8 aa, Mus sp.
IDPSISYT

SEQ ID NO: 178 - protein, 8 aa, Mus sp.
INPDSSTI

SEQ ID NO: 179 - protein, 7 aa, Mus sp.
ARGAFDY

SEQ ID NO: 180 - protein, 10 aa, Mus sp.
SRDGHGEGDY

SEQ ID NO: 181 - protein, 15 aa, Mus sp.
TREGDGYYPYYAMDY

SEQ ID NO: 182 - protein, 13 aa, Mus sp.
VRISTETNWYFDV

SEQ ID NO: 183 - protein, 11 aa, Mus sp.
ARDSYGPYLDY

SEQ ID NO: 184 - protein, 14 aa, Mus sp.
TKSGGDFVGYALDY

SEQ ID NO: 185 - protein, 9 aa, Mus sp.
VRTYGYYES

SEQ ID NO: 186 - protein, 6 aa, Mus sp.
TNEGTF

SEQ ID NO: 187 - protein, 13 aa, Mus sp.
ARGLYYDYDWFAY

SEQ ID NO: 188 - protein, 12 aa, Mus sp.
AKQNRYDGIFDY

SEQ ID NO: 189 - protein, 11 aa, Mus sp.
ARNYGNYRFAY

SEQ ID NO: 190 - protein, 13 aa, Mus sp.
ARSPYSNYDYFDY

SEQ ID NO: 191 - protein, 12 aa, Mus sp.
ARQLGLPYAMDY

SEQ ID NO: 192 - protein, 12 aa, Mus sp.
TREDGYYSTMDY

SEQ ID NO: 193 - protein, 12 aa, Mus sp.
ARSYNYDVVFAY

SEQ ID NO: 194 - protein, 12 aa, Mus sp.
ARSGGNYGWFAY

SEQ ID NO: 195 - protein, 12 aa, Mus sp.
ARYGGTGYGMDY

SEQ ID NO: 196 - protein, 10 aa, Mus sp.
ARRGLHYFDY

SEQ ID NO: 197 - protein, 9 aa, Mus sp.
AGPHWYLDV

SEQ ID NO: 198 - protein, 12 aa, Mus sp.
SRQNWDGGYFDV

SEQ ID NO: 199 - protein, 12 aa, Mus sp.
AKRDYSNYGLAY

SEQ ID NO: 200 - protein, 11 aa, Mus sp.
AKYDGYYLFDY

SEQ ID NO: 201 - protein, 12 aa, Mus sp.
ARISVMVTTYFD

SEQ ID NO: 202 - protein, 10 aa, Mus sp.
ALYYGNYVNF

SEQ ID NO: 203 - protein, 11 aa, Mus sp.
ARYGNLLDLDY

SEQ ID NO: 204 - protein, 13 aa, Mus sp.
ARYYSFPYWYFDV

SEQ ID NO: 205 - protein, 14 aa, Mus sp.
ARQGDRYDGYVMDY

SEQ ID NO: 206 - protein, 17 aa, Mus sp.
ARIPYYYDGSYVYYFDY

SEQ ID NO: 207 - protein, 8 aa, Mus sp.
GRPNFFDY

SEQ ID NO: 208 - protein, 11 aa, Mus sp.
ARGGWDDWFAY

SEQ ID NO: 209 - protein, 9 aa, Mus sp.
ARGGPPFAY

SEQ ID NO: 210 - protein, 14 aa, Mus sp.
ASYYAVENYAWFPY

SEQ ID NO: 211 - protein, 13 aa, Mus sp.
TTDYDEGKYYFDY

SEQ ID NO: 212 - protein, 14 aa, Mus sp.
IRRGAYYNKEAMDS

SEQ ID NO: 213 - protein, 15 aa, Mus sp.
ARDLGLNWDPPWFAY

SEQ ID NO: 214 - protein, 11 aa, Mus sp.
ARRGATGFFDY

SEQ ID NO: 215 - protein, 5 aa, Mus sp.
ARDGD

SEQ ID NO: 216 - protein, 8 aa, Mus sp.
ASLPLVDY

SEQ ID NO: 217 - protein, 14 aa, Mus sp.
KSSQSLLYSNGKTY

SEQ ID NO: 218 - protein, 16 aa, Mus sp.
RSSQNIVHSNGNTYLE

SEQ ID NO: 219 - protein, 11 aa, Mus sp.
QSLVHSNGNTY

SEQ ID NO: 220 - protein, 11 aa, Mus sp.
QSLLDSDGKTY

SEQ ID NO: 221 - protein, 15 aa, Mus sp.
KASQSVDYDGDSYMN

SEQ ID NO: 222 - protein, 17 aa, Mus sp.
KSSQSLLYSNNQKNYLA

SEQ ID NO: 223 - protein, 14 aa, Mus sp.
KSSQSLLDSDGKTY

SEQ ID NO: 224 - protein, 15 aa, Mus sp.
KASQSVDYDGDSYMN

SEQ ID NO: 225 - protein, 17 aa, Mus sp.
KSSQSLLYSSNQKNYVA

SEQ ID NO: 226 - protein, 17 aa, Mus sp.
TSSQSLLFSSNQKNYLA

SEQ ID NO: 227 - protein, 11 aa, Mus sp.
KASDHINNWLA

SEQ ID NO: 228 - protein, 11 aa, Mus sp.
HASQNINVWLS

SEQ ID NO: 229 - protein, 11 aa, Mus sp.
HASQDISSNIG

SEQ ID NO: 230 - protein, 11 aa, Mus sp.
KASQDINSYLS

SEQ ID NO: 231 - protein, 17 aa, Mus sp.
SASQSLLYSSNQKNYLA

SEQ ID NO: 232 - protein, 12 aa, Mus sp.
SASSSVSSIYLH

SEQ ID NO: 233 - protein, 13 aa, Mus sp.
QASEGVSFAGSSL

SEQ ID NO: 234 - protein, 16 aa, Mus sp.
RSSQNIVHSNGNTYLE

SEQ ID NO: 235 - protein, 6 aa, Mus sp.
QDISNY

SEQ ID NO: 236 - protein, 10 aa, Mus sp.
KSVSSSGYSY

SEQ ID NO: 237 - protein, 6 aa, Mus sp.
EDIYNR

SEQ ID NO: 238 - protein, 11 aa, Mus sp.
QSLANSYGNTY

SEQ ID NO: 239 - protein, 11 aa, Mus sp.
QSLLDSDGKTY

SEQ ID NO: 240 - protein, 11 aa, Mus sp.
QSIVHCNGNTY

SEQ ID NO: 241 - protein, 6 aa, Mus sp.
ENIYIN

SEQ ID NO: 242 - protein, 12 aa, Mus sp.
QSLLNSGNQKNY

SEQ ID NO: 243 - protein, 9 aa, Mus sp.
TGAVTTSYY

SEQ ID NO: 244 - protein, 6 aa, Mus sp.
ENGDNY

SEQ ID NO: 245 - protein, 11 aa, Mus sp.
QSLVHSNGNTY

SEQ ID NO: 246 - protein, 11 aa, Mus sp.
QTIVHRSGSTY

SEQ ID NO: 247 - protein, 6 aa, Mus sp.
EDIYNR

SEQ ID NO: 248 - protein, 6 aa, Mus sp.
QSINNY

SEQ ID NO: 249 - protein, 5 aa, Mus sp.
SSVSY

SEQ ID NO: 250 - protein, 10 aa, Mus sp.
ESVDSYGNSF

SEQ ID NO: 251 - protein, 12 aa, Mus sp.
QSLLYNSNQKNY

SEQ ID NO: 252 - protein, 6 aa, Mus sp.
ENIYST

SEQ ID NO: 253 - protein, 10 aa, Mus sp.
QSLLDSDGKT

SEQ ID NO: 254 - protein, 6 aa, Mus sp.
QNVGTS

SEQ ID NO: 255 - protein, 7 aa, Mus sp.
LVSKLDS

SEQ ID NO: 256 - protein, 7 aa, Mus sp.
KVSNRFS

SEQ ID NO: 257 - protein, 7 aa, Mus sp.
KVSNRFS

SEQ ID NO: 258 - protein, 7 aa, Mus sp.
LVSKLAS

SEQ ID NO: 259 - protein, 7 aa, Mus sp.
GASNLES

SEQ ID NO: 260 - protein, 7 aa, Mus sp.
WASTRES

SEQ ID NO: 261 - protein, 7 aa, Mus sp.
LVSKLDS

SEQ ID NO: 262 - protein, 7 aa, Mus sp.
SASDLES

SEQ ID NO: 263 - protein, 7 aa, Mus sp.
WASTRES

SEQ ID NO: 264 - protein, 7 aa, Mus sp.
WASTRES

SEQ ID NO: 265 - protein, 7 aa, Mus sp.
GATSLET

SEQ ID NO: 266 - protein, 7 aa, Mus sp.
NKASLHT

SEQ ID NO: 267 - protein, 7 aa, Mus sp.
HGTNLED

SEQ ID NO: 268 - protein, 7 aa, Mus sp.
RAARLVD

SEQ ID NO: 269 - protein, 7 aa, Mus sp.
WASTRES

SEQ ID NO: 270 - protein, 7 aa, Mus sp.
RTSNLAS

SEQ ID NO: 271 - protein, 7 aa, Mus sp.
RASNLES

SEQ ID NO: 272 - protein, 7 aa, Mus sp.
KVSNRFS

SEQ ID NO: 273 - protein, 7 aa, Mus sp.
YTSRLHS

SEQ ID NO: 274 - protein, 7 aa, Mus sp.
LASNLES

SEQ ID NO: 275 - protein, 7 aa, Mus sp.
GATSLET

SEQ ID NO: 276 - protein, 7 aa, Mus sp.
GISNRFS

SEQ ID NO: 277 - protein, 7 aa, Mus sp.
MVSKLAS

SEQ ID NO: 278 - protein, 7 aa, Mus sp.
KVSNRFS

SEQ ID NO: 279 - protein, 7 aa, Mus sp.
AATNLAD

SEQ ID NO: 280 - protein, 7 aa, Mus sp.
WASTRES

SEQ ID NO: 281 - protein, 7 aa, Mus sp.
GTNYRAP

SEQ ID NO: 282 - protein, 7 aa, Mus sp.
GASNRYT

SEQ ID NO: 283 - protein, 7 aa, Mus sp.
KVSNRFS

SEQ ID NO: 284 - protein, 7 aa, Mus sp.
KVSNRFS

SEQ ID NO: 285 - protein, 7 aa, Mus sp.
GATSLET

SEQ ID NO: 286 - protein, 7 aa, Mus sp.
YASQSIS

SEQ ID NO: 287 - protein, 7 aa, Mus sp.
ATSNLAS

SEQ ID NO: 288 - protein, 7 aa, Mus sp.
LASNLES

SEQ ID NO: 289 - protein, 7 aa, Mus sp.
WASTRES

SEQ ID NO: 290 - protein, 7 aa, Mus sp.
TGTNLAD

SEQ ID NO: 291 - protein, 7 aa, Mus sp.
LVSKLDS

SEQ ID NO: 292 - protein, 7 aa, Mus sp.
SASYRYS

SEQ ID NO: 293 - protein, 9 aa, Mus sp.
VQGTHFPYT

SEQ ID NO: 294 - protein, 9 aa, Mus sp.
FQGSHVPYT

SEQ ID NO: 295 - protein, 9 aa, Mus sp.
SQITHVPLT

SEQ ID NO: 296 - protein, 9 aa, Mus sp.
WQGTHFPHT

SEQ ID NO: 297 - protein, 9 aa, Mus sp.
QQSKEDPPT

SEQ ID NO: 298 - protein, 8 aa, Mus sp.
QQYYRYRT

SEQ ID NO: 299 - protein, 9 aa, Mus sp.
WQGTHFPQT

SEQ ID NO: 300 - protein, 9 aa, Mus sp.
HQSNEDPFT

SEQ ID NO: 301 - protein, 9 aa, Mus sp.
QQYYRYPYT

SEQ ID NO: 302 - protein, 10 aa, Mus sp.
QQYYSYPPWT

SEQ ID NO: 303 - protein, 9 aa, Mus sp.
QQYWSTPLT

SEQ ID NO: 304 - protein, 9 aa, Mus sp.
QQGQSYPLT

SEQ ID NO: 305 - protein, 9 aa, Mus sp.
VQYAQFPYT

SEQ ID NO: 306 - protein, 9 aa, Mus sp.
LQYDEFPYT

SEQ ID NO: 307 - protein, 10 aa, Mus sp.
QQYYSYPPYT

SEQ ID NO: 308 - protein, 9 aa, Mus sp.
QQWSGYPFT

SEQ ID NO: 309 - protein, 9 aa, Mus sp.
MQSMEDPFT

SEQ ID NO: 310 - protein, 9 aa, Mus sp.
FQGSHVPFT

SEQ ID NO: 311 - protein, 9 aa, Mus sp.
QQTNTLPRT

SEQ ID NO: 312 - protein, 9 aa, Mus sp.
QHSRALPLT

SEQ ID NO: 313 - protein, 9 aa, Mus sp.
QQYWSPPWT

SEQ ID NO: 314 - protein, 9 aa, Mus sp.
LQGTHQPWT

SEQ ID NO: 315 - protein, 9 aa, Mus sp.
WQGTHFPHT

SEQ ID NO: 316 - protein, 9 aa, Mus sp.
FQGSHVPYT

SEQ ID NO: 317 - protein, 9 aa, Mus sp.
QHFWGTPYT

SEQ ID NO: 318 - protein, 9 aa, Mus sp.
QNDYNYPLT

SEQ ID NO: 319 - protein, 9 aa, Mus sp.
ALWYNNHWV

SEQ ID NO: 320 - protein, 8 aa, Mus sp.
GQSYRYPT

SEQ ID NO: 321 - protein, 9 aa, Mus sp.
SQSTHVPLT

SEQ ID NO: 322 - protein, 9 aa, Mus sp.
FQGSHVPLT

SEQ ID NO: 323 - protein, 9 aa, Mus sp.
QQYWSTPLT

SEQ ID NO: 324 - protein, 9 aa, Mus sp.
QQSNSWPLT

SEQ ID NO: 325 - protein, 10 aa, Mus sp.
QQWSSNPQYT

SEQ ID NO: 326 - protein, 9 aa, Mus sp.
QQNNEDPYT

SEQ ID NO: 327 - protein, 9 aa, Mus sp.
QQYDSYPPT

SEQ ID NO: 328 - protein, 8 aa, Mus sp.
QHFYGTVT

SEQ ID NO: 329 - protein, 9 aa, Mus sp.
WQGTHFPQT

SEQ ID NO: 330 - protein, 9 aa, Mus sp.
EQYKSYPYT

SEQ ID NO: 331 - protein, 119 aa, Artificial Sequence
  source: /note=“Description of Artificial Sequence Synthetic polypeptide”
EVQLVESGGGLVQPGGSLRLSCAASGFTFSSYGMNWVRQAPGKGLEWVSYISSSTVTTYYADSVKGRFTISRDNAKNSLYLQMNSLRDEDTAVYYCSRQNWDGGYFDVWGAGTTVTVSS

SEQ ID NO: 332 - protein, 112 aa, Artificial Sequence
  source: /note=“Description of Artificial Sequence Synthetic polypeptide”
DIVMTQSPLSLPVTPGEPASISCRASKSVSSSGYSYMHWYLQKPGQSPQLLIYLASNLESGVPDRFSGSGSGTDFTLKISRVEAEDVGVYYCQHSRALPLTFGAGTKLELKR

SEQ ID NO: 333 - protein, 727 aa, Artificial Sequence [sequence omitted]

SEQ ID NO: 334 - DNA, 2184 bp, Artificial Sequence
  source: /note=“Description of Artificial Sequence Synthetic polynucleotide”
atgtacaggatgcaactcctgtcttgcattgcactaagtcttgcacttgtcacgaattcgagcgagggcaggaagacctggcggcgccggggtcagcagccgcctcctcccccgcggaccgaggcggcgccggcggccggacagcccgtggagagcttcccgctggacttcacggccgtggagggtaacatggacagcttcatggcgcaagtcaagagcctggcgcagtccctgtacccctgctccgcgcagcagctcaacgaggacctgcgcctgcacctcctactcaacacctcggtgacctgcaacgacggcagccccgccggctactacctgaaggagtccaggggcagccggcggtggctcctcttcctggaaggcggctggtactgcttcaaccgcgagaactgcgactccagatacgacaccatgcggcgcctcatgagctcccgggactggccgcgcactcgcacaggcacagggatcctgtcctcacagccggaggagaacccctactggtggaacgcaaacatggtcttcatcccctactgctccagtgatgtttggagcggggcttcatccaagtctgagaagaacgagtacgccttcatgggcgccctcatcatccaggaggtggtgcgggagcttctgggcagagggctgagcggggccaaggtgctgctgctggccgggagcagcgcggggggcaccggggtgctcctgaatgtggaccgtgtggctgagcagctggagaagctgggctacccagccatccaggtgcgaggcctggctgactccggctggttcctggacaacaagcagtatcgccacacagactgcgtcgacacgatcacgtgcgcgcccacggaggccatccgccgtggcatcaggtactggaacggggtggtcccggagcgctgccgacgccagttccaggagggcgaggagtggaactgcttctttggctacaaggtctacccgaccctgcgctgccctgtgttcgtggtgcagtggctgtttgacgaggcacagctgacggtggacaacgtgcacctgacggggcagccggtgcaggagggcctgcggctgtacatccagaacctcggccgcgagctgcgccacacactcaaggacgtgccggccagctttgcccccgcctgcctctcccatgagatcatcatccggagccactggacggatgtccaggtgaaggggacgtcgctgccccgagcactgcactgctgggacaggagcctccatgacagccacaaggccagcaagacccccctcaagggctgccccgtccacctggtggacagctgcccctggccccactgcaacccctcatgccccaccgtccgagaccagttcacggggcaagagatgaacgtggcccagttcctcatgcacatgggcttcgacatgcagacggtggcccagccgcagggactggagcccagtgagctgctggggatgctgagcaacggaagcatatcggccatggttagatctgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga